(19) United States
(12) Patent Application Publication
(10) Pub. No.: US 2023/0332193 A1
(43) Pub. Date: Oct. 19, 2023

(54) FLAVIN-DEPENDENT OXIDASES HAVING CANNABINOID SYNTHASE ACTIVITY

(71) Applicant: GENOMATICA, INC., San Diego, CA (US)

(72) Inventors: Andreas W. SCHIRMER, San Diego, CA (US); Deqiang ZHANG, San Diego, CA (US); Jamison Parker HUDDLESTON, San Diego, CA (US)

(21) Appl. No.: 18/043,258

(22) PCT Filed: Sep. 1, 2021
(86) PCT No.: PCT/US2021/048698

§ 371 Date: Feb. 27, 2023

Related U.S. Application Data
(60) Provisional application No. 63/138,109, filed on Jan. 15, 2021.
(60) Provisional application No. 63/074,227, filed on Sep. 3, 2020.

(51) Int. Cl. C12P 17/06; C12N 9/04; C12N 9/10; C12N 9/88; C12N 15/52

(52) U.S. Cl. CPC C12P 17/06; C12N 9/0006; C12N 9/1029; C12Y 203/01206; C12N 9/88; C12Y 404/01026; C12N 9/1085; C12Y 205/0101; C12N 15/52

(57) ABSTRACT

The disclosure relates to a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid. The disclosure also relates to a nucleic acid, an expression construct, and an engineered cell for making the non-natural flavin-dependent oxidase. Also provided are compositions comprising the non-natural flavin-dependent oxidase; isolated non-natural flavin-dependent oxidase and methods of making the same; cell extracts comprising the non-natural flavin-dependent oxidase; and methods of making cannabinoids.

### SEQUENCE LISTING

The instant application contains a Sequence Listing which has been submitted electronically in ASCII format and is hereby incorporated by reference in its entirety. Said ASCII copy, created on Sep. 1, 2021, is named 0171-0002WO1_SL.txt and is 75,215 bytes in size.

### FIELD OF THE INVENTION

The disclosure relates to a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid. The disclosure also relates to a nucleic acid, an expression construct, and an engineered cell for making the non-natural flavin-dependent oxidase. Also provided are compositions comprising the non-natural flavin-dependent oxidase; isolated non-natural flavin-dependent oxidase and methods of making the same; cell extracts comprising the non-natural flavin-dependent oxidase; and methods of making cannabinoids. The disclosure further relates to a composition comprising: a flavin-dependent oxidase comprising any of SEQ ID NOs:1-6; and a cannabinoid, and a method of making a cannabinoid comprising contacting CBGA, CBGOA, CBGVA, or CBG with a flavin-dependent oxidase comprising any of SEQ ID NOs:1-6.

### BACKGROUND

Cannabinoids constitute a varied class of chemicals, typically prenylated polyketides derived from fatty acid and isoprenoid precursors, that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as Cannabis, and synthetic cannabinoids, can have activity on cannabinoid receptors and elicit biological responses. Recently, cannabinoids have drawn significant scientific interest in their potential to treat a wide array of disorders, including insomnia, chronic pain, epilepsy, and post-traumatic stress disorder (Babson et al. (2017), Curr Psychiatry Rep 19:23; Romero-Sandoval et al. (2017) Curr Rheumatol Rep 19:67; O'Connell et al. (2017) Epilepsy Behav 70:341-348; Zir-Aviv et al. (2016) Behav Pharmacol 27:561-569). Cannabinoid research and development as therapeutic tools requires production in large quantities and at high purity. However, purifying individual cannabinoid compounds from C. sativa can be time-consuming and costly, and it can be difficult to isolate a pure sample of a compound of interest. Thus, engineered cells can be a useful alternative for the production of a specific cannabinoid or cannabinoid precursor.

### SUMMARY OF THE INVENTION

The present disclosure relates to flavin-dependent oxidases that have cannabinoid synthase activity.

In some embodiments, the disclosure provides a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid. In some embodiments, the flavin-dependent oxidase comprises at least 70%, at least 80%, at least 85%, or at least 90% sequence identity to SEQ ID NO:1, and wherein the at least one amino acid variation comprises a substitution at position V136, S137, T139, L144, Y249, F313, Q353, or a combination thereof, wherein the amino acid position corresponds to SEQ ID NO:1. In some embodiments, the non-natural flavin-dependent oxidase comprises at least 70%, at least 80%, at least 85%, or at least 90% sequence identity to SEQ ID NO:3, and wherein the at least one amino acid variation comprises: a substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3. In some embodiments, the non-natural flavin-dependent oxidase comprises at least 70% sequence identity to SEQ ID NO:3, e.g., at least 80% sequence identity, at least 85% sequence identity, or at least 90% sequence identity to SEQ ID NO: 3, and wherein the at least one amino acid variation comprises a deletion of about 5 to about 50 amino acid residues at an N-terminus of SEQ ID NO:3, optionally comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3. In some embodiments, the non-natural flavin-dependent oxidase comprises at least 70%, at least 80%, at least 85%, or at least 90% sequence identity to SEQ ID NO:19 or 20, optionally comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

In some embodiments, the non-natural flavin-dependent oxidase is a berberine bridge enzyme (BBE)-like enzyme. In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG).

In some embodiments, the non-natural flavin-dependent oxidase has at least 70% sequence identity to SEQ ID NO:1, 3, 19, or 20. In some embodiments, the non-natural flavin-dependent oxidase has at least 80% sequence identity to SEQ ID NO:1, 3, 19, or 20.

In some embodiments, the non-natural flavin-dependent oxidase does not comprise a disulfide bond. In some embodiments, the non-natural flavin-dependent oxidase is not glycosylated. In some embodiments, the non-natural flavin-dependent oxidase comprises a monovalently bound FAD cofactor. In some embodiments, the non-natural flavin-dependent oxidase comprises a bivalently bound FAD cofactor.

In some embodiments, the non-natural flavin-dependent oxidase is capable of oxidative cyclization of a prenylated aromatic compound into a cannabinoid at about pH 7.5. In some embodiments, catalytic activity of the non-natural flavin-dependent oxidase is substantially the same from about pH 5 to about pH 8.

In some embodiments, the at least one amino acid variation comprises a substitution, deletion, insertion, or a combination thereof. In some embodiments, the non-natural flavin-dependent oxidase has at least 90% sequence identity to SEQ ID NO:1. In some embodiments, the non-natural flavin-dependent oxidase has at least 95% sequence identity to SEQ ID NO:1.

In some embodiments, the non-natural flavin-dependent oxidase comprises a variation at amino acid position V136, S137, T139, L144, Y249, F313, Q353, or a combination thereof, wherein the amino acid position corresponds to SEQ ID NO:1. In some embodiments, the variation comprises an amino acid substitution selected from V136C, S137P, T139V, L144H, Y249H, F313A, Q353N, or a combination thereof. In some embodiments, the variation comprises a T139V substitution.

In some embodiments, the non-natural flavin-dependent oxidase converts CBGA to cannabichromenic acid (CBCA), tetrahydrocannabinolic acid (THCA), or both. In some embodiments, the non-natural flavin-dependent oxidase converts CBGA to CBCA at about pH 4 to about pH 9. In some embodiments, the non-natural flavin-dependent oxidase converts CBGOA to cannabiorcichromenic acid (CBCOA). In some embodiments, the non-natural flavin-dependent oxidase converts CBGVA to cannabichromevarinic acid (CBCVA). In some embodiments, the non-natural flavin-dependent oxidase converts CBG to cannabichromene (CBC).

In some embodiments, the non-natural flavin-dependent oxidase has at least 90% sequence identity to SEQ ID NO:3. In some embodiments, the non-natural flavin-dependent oxidase has at least 95% sequence identity to SEQ ID NO:3. In some embodiments, the non-natural flavin-dependent oxidase has at least 90% sequence identity to SEQ ID NO:19 or 20. In some embodiments, the non-natural flavin-dependent oxidase has at least 95% sequence identity to SEQ ID NO:19 or 20. In some embodiments, the non-natural flavin-dependent oxidase comprises a variation at amino acid position W58, M101, L104, I160, G161, A163, V167, L168, A171, N267, L269, I271, Y273, Q275, L283, C285, E287, V323, V336, A338, G340, L342, E370, V372, A398, N400, H402, D404, V436, T438, or a combination thereof, wherein the amino acid position corresponds to SEQ ID NO:3. In some embodiments, the variation comprises an amino acid substitution selected from W58Q, W58H, W58K, W58G, W58V, M101A, M101S, M101F, M101Y, L104M, L104H, I160V, G161C, G161A, G161Q, G161L, A163G, V167F, L168S, L168G, A171Y, A171F, N267V, N267M, N267L, L269M, L269T, L269A, L269R, I271H, I271R, Y2731, Y273R, Q275K, Q275R, A281R, L283V, C285L, E287H, E287L, V323F, V323Y, V336F, A338I, G340L, L342Y, E370M, E370Q, V372A, V372E, V372I, V372L, V372T, V372C, A398E, A398V, N400W, H402T, H402I, H402V, H402A, H402M, H402Q, D404S, D404T, D404A, V436L, T438A, T438Y, T438F, or a combination thereof, wherein the amino acid position corresponds to SEQ ID NO:3. In some embodiments, the variation comprises an amino acid substitution selected from T438A, T438Y, N400W, D404A, or a combination thereof, wherein the amino acid position corresponds to SEQ ID NO:3.

In some embodiments, the variation in the non-natural flavin-dependent oxidase comprises an amino acid substitution at position D404 and an amino acid substitution at position L269, Y273, Q275, L283, C285, V323, E370, V372, N400, H402, T438, or a combination thereof, wherein the amino acid position corresponds to SEQ ID NO:3. In some embodiments, the variation comprises D404A and one of: L269R, L269T, Q275R, Y273R, L283V, C285L, V323F, V323Y, E370M, E370Q, V372I, N400W, H402A, H402I, H402M, H402T, H402V, T438A, T438F, or T438Y.

In some embodiments, the variation in the non-natural flavin-dependent oxidase comprises an amino acid substitution at position D404, an amino acid substitution at position T438, and an amino acid position L269, Y273, Q275, L283, C285, V323, E370, V372, N400, H402, T438, or a combination thereof, wherein the amino acid position corresponds to SEQ ID NO:3.

In some embodiments, the variation comprises: a) D404A, T438F, and N400W; b) D404A, T438F, and V323F; c) D404A, T438F, and V323Y; d) D404A, T438F, and E370M; e) D404A, T438F, and H402I; f) D404A, T438F, and E370Q; g) D404A, T438F, and C285L; h) T438F, N400W, and D404S; i) T438F, V323Y, and D404S; j) T438F, H402I, and D404S; k) T438F, E370Q, and D404S; l) D404A, T438F, V372I, and N400W; m) D404A, T438F, V323Y, and N400W; n) D404A, T438F, E370Q, and N400W; o) D404A, T438F, V323Y, and E370M; p) D404A, T438F, E370M, and N400W; q) D404A, T438F, V323F, and H402I; r) D404A, T438F, C285L, and N400W; s) D404A, T438F, V323F, and N400W; t) D404A, T438F, E370Q, and H402T; u) D404A, T438F, N400W, and H402T; v) D404A, T438F, V323F, and H402T; w) D404A, T438F, C285L, and V323F; x) D404A, T438F, L283V, and N400W; y) D404A, T438F, V323F, and E370M; z) D404A, T438F, Q275R, and N400W; aa) D404A, T438F, V323Y, and H402T; bb) D404A, T438F, V323F, and V372I; cc) D404A, T438F, C285L, and V323Y; dd) D404A, T438F, E370Q, and H402I; ee) D404A, T438F, V323Y, and E370Q; ff) D404A, T438F, Y273R, and V323Y; gg) D404A, T438F, Y273R, and N400W; hh) D404A, T438F, Y273R, and V323F; ii) D404A, T438F, E370M, and H402T; jj) D404A, T438F, L269T, and N400W; kk) D404A, T438F, Q275R, and V323Y; ll) D404A, T438F, V323Y, and H402I; mm) D404A, T438F, V323F, and E370Q; nn) D404A, T438F, Y273R, and Q275R; oo) D404A, T438F, C285L, and E370Q; pp) D404A, T438F, L283V, and V323Y; qq) D404A, T438F, Y273R, and H402I; rr) D404A, T438F, L269T, and E370M; ss) D404A, T438F, C285L, and H402T; tt) D404A, T438F, L269R, and N400W; uu) D404A, T438F, Y273R, and C285L; vv) D404A, T438F, L283V, and H402I; ww) D404A, T438F, Q275R, and E370Q; xx) D404A, T438F, V372I, and H402I; yy) D404A, T438F, L283V, and E370Q; or zz) D404A, T438F, V372I, and H402T; wherein the amino acid position corresponds to SEQ ID NO:3.

In some embodiments, the variation in the non-natural flavin-dependent oxidase comprises D404A, N400W, and V323Y. In some embodiments, the variation in the flavin-dependent oxidase comprises D404A, T438F, N400W, and V323Y. In some embodiments, the variation comprises an amino acid substitution at position D404, an amino acid substitution at position T438, an amino acid substitution at position N400, an amino acid substitution at position V323, and an amino acid substitution at position L269, I271, Q275, A281, L283, C285, E370, V372, H402, or a combination thereof. In some embodiments, the variation comprises D404A, T438F, N400W, V323Y, and one or more of: L269M, I271H, Q275R, A281R, L283S, C285L, E370M, E370Q, V372I, and H402T.

In some embodiments, the variation comprises: (a) D404A, T438F, N400W, V323Y, and E370Q; (b) D404A, T438F, N400W, V323Y, and V372I; (c) D404A, T438F, N400W, V323Y, and L269M; (d) D404A, T438F, N400W, V323Y, and C285L; (e) D404A, T438F, N400W, V323Y, and A281R; (f) D404A, T438F, N400W, V323Y, I271H, and E370Q; (g) D404A, T438F, N400W, V323Y, E370Q, and V372I; (h) D404A, T438F, N400W, V323Y, L269M, and E370Q; (i) D404A, T438F, N400W, V323Y, C285L, and E370Q; (j) D404A, T438F, N400W, V323Y, Q275R, and E370Q; (k) D404A, T438F, N400W, V323Y, L283S, and E370Q; (l) D404A, T438F, N400W, V323Y, A281R, and C285L; (m) D404A, T438F, N400W, V323Y, Q275R, and V372I; (n) D404A, T438F, N400W, V323Y, C285L, and E370M; (o) D404A, T438F, N400W, V323Y, L269M, and V372I; (p) D404A, T438F, N400W, V323Y, Q275R, and C285L; (q) D404A, T438F, N400W, V323Y, I271H, and L283S; (r) D404A, T438F, N400W, V323Y, Q275R, and A281R; (s) D404A, T438F, N400W, V323Y, L269M, and I271H; (t) D404A, T438F, N400W, V323Y, I271H, and E370M; (u) D404A, T438F, N400W, V323Y, I271H, and C285L; (v) D404A, T438F, N400W, V323Y, A281R, and V372I; (w) D404A, T438F, N400W, V323Y, E370M, and V372I; (x) D404A, T438F, N400W, V323Y, L269M, and Q275R; (y) D404A, T438F, N400W, V323Y, C285L, and V372I; (z) D404A, T438F, N400W, V323Y, V372I, and H402T; (aa) D404A, T438F, N400W, V323Y, L269M, and E370M; (bb) D404A, T438F, N400W, V323Y, Q275R, and E370M; (cc) D404A, T438F, N400W, V323Y, A281R, and E370Q; or (dd) D404A, T438F, N400W, V323Y, A281R, and L283S.

In some embodiments, the non-natural flavin-dependent oxidase does not comprise a variation at any of amino acid positions Y374, Y435, and N437, wherein the amino acid position corresponds to SEQ ID NO:3.

In some embodiments, the variation in the non-natural flavin-dependent oxidase comprises a deletion of about 5 to about 50 amino acid residues at the N-terminus of SEQ ID NO:3. In some embodiments, the variation in the non-natural flavin-dependent oxidase comprises a deletion of about 10 to about 40 amino acid residues at the N-terminus of SEQ ID NO:3. In some embodiments, the variation in the non-natural flavin-dependent oxidase comprises a deletion of about 12 to about 35 amino acid residues at the N-terminus of SEQ ID NO:3. In some embodiments, the variation in the non-natural flavin-dependent oxidase comprises a deletion of about 14 to about 30 amino acid residues at the N-terminus of SEQ ID NO:3.

In some embodiments, the non-natural flavin-dependent oxidase converts CBGA to cannabichromenic acid (CBCA), tetrahydrocannabinolic acid (THCA), or both. In some embodiments, the non-natural flavin-dependent oxidase converts CBGA to CBCA at about pH 4 to about pH 9. In some embodiments, the non-natural flavin-dependent oxidase converts CBGOA to cannabiorcichromenic acid (CBCOA). In some embodiments, the non-natural flavin-dependent oxidase converts CBGOA to CBCOA at about pH 4 to about pH 9. In some embodiments, the non-natural flavin-dependent oxidase converts CBGVA to cannabichromevarinic acid (CBCVA). In some embodiments, the non-natural flavin-dependent oxidase converts CBGVA to CBCVA at about pH 4 to about pH 9. In some embodiments, the non-natural flavin-dependent oxidase converts CBG to cannabichromene (CBC) at about pH 4 to about pH 9.

In some embodiments, the non-natural flavin-dependent oxidase converts CBGO to cannabiorcichromene. In some embodiments, the non-natural flavin-dependent oxidase converts CBGV to cannabichromevarin. In some embodiments, the non-natural flavin-dependent oxidase further comprises an affinity tag, a purification tag, a solubility tag, or a combination thereof.

In some embodiments, the disclosure provides a polynucleotide comprising a nucleic acid sequence encoding the non-natural flavin-dependent oxidase described herein. In some embodiments, the disclosure provides a polynucleotide comprising: (a) a nucleic acid sequence encoding a polypeptide having at least 80% sequence identity to SEQ ID NO:1, 3, 19, or 20; and (b) a heterologous regulatory element operably linked to the nucleic acid sequence, wherein: (i) the polypeptide having at least 80% identity to SEQ ID NO:1 comprises an amino acid substitution at position V136, S137, T139, L144, Y249, F313, Q353, or a combination thereof; or (ii) the polypeptide having at least 80% identity to SEQ ID NO:3 comprises an amino acid substitution at position W58, M101, L104, I160, G161, A163, V167, L168, A171, N267, L269, I271, Y273, Q275, L283, C285, E287, V323, V336, A338, G340, L342, E370, V372, A398, N400, H402, D404, V436, T438, or a combination thereof, or (iii) the polypeptide having at least 80% sequence identity to SEQ ID NO:3 comprises a deletion of about 5 to about 50 amino acid residues at an N-terminus of SEQ ID NO:3, and optionally further comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or (iv) wherein the polypeptide having at least 70%, at least 80%, at least 85%, or at least 90% sequence identity to SEQ ID NO:19 or 20 optionally comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3. In some embodiments, the disclosure provides an expression construct comprising the polynucleotide described herein.

In some embodiments, the disclosure provides an engineered cell comprising the non-natural flavin-dependent oxidase, the polynucleotide, the expression construct, or a combination thereof.

In some embodiments, the engineered cell further comprises a cannabinoid biosynthesis pathway enzyme. In some embodiments, the cannabinoid biosynthesis pathway enzyme comprises olivetol synthase (OLS), olivetolic acid cyclase (OAC), prenyltransferase, or a combination thereof.

In some embodiments, the OLS comprises an amino acid substitution at position A125, S126, D185, M187, L190, G204, G209, D210, G211, G249, G250, L257, F259, M331, S332, or a combination thereof, wherein the position corresponds to SEQ ID NO:7. In some embodiments, the amino acid substitution is selected from A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M1871, M187K, M187R, L190G, L190A, L190S, L190P, L190C, L190T, L190D, L190N, L190E, L190Q, L190H, L190V, L190M, L190I, L190K, L190R, G204A, G204C, G204P, G204V, G204L, G2041, G204M, G204F, G204W, G204S, G204T, G204Y, G204H, G204N, G204Q, G204D, G204E, G204K, G204R, G209A, G209C, G209P, G209V, G209L, G2091, G209M, G209F, G209W, G209S, G209T, G209Y, G209H, G209N, G209Q, G209D, G209E, G209K, G209R, D210A, D210C, D210P, D210V, D210L, D2101, D210M, D210F, D210W, D210S, D210T, D210Y, D210H, D210N, D210Q, D210E, D210K, D210R, G211A, G211C, G211P, G211V, G211L, G211I, G211M, G211F, G211W, G211S, G211T, G211Y, G211H, G211N, G211Q, G211D, G211E, G211K, G211R, G249A, G249C, G249P, G249V, G249L, G2491, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M331I, M331K, M331R, S332G, S332A, and a combination thereof.

In some embodiments, the OAC comprises an amino acid substitution at position L9, F23, V59, V61, V66, E67, 169, Q70, I73, I74, V79, G80, F81, G82, D83, R86, W89, L92, I94, V46, T47, Q48, K49, N50, K51, V46, T47, Q48, K49, N50, K51, or a combination thereof, wherein the position corresponds to SEQ ID NO:8.

In some embodiments, the prenyltransferase comprises an amino acid substitution at position V45, F121, T124, Q159, M160, Y173, S212, A230, T267, Y286, Q293, R294, L296, F300, or a combination thereof, wherein the position corresponds to SEQ ID NO:9. In some embodiments, the amino acid substitution is selected from V451, V45T, F121V, T124K, T124L, Q159S, M160L, M160S, Y173D, Y173K, Y173P, Y173Q, S212H, A230S, T267P, Y286V, Q293H, R294K, L296K, L296L, L296M, L296Q, F300Y, and a combination thereof.

In some embodiments, the engineered cell further comprises a geranyl pyrophosphate (GPP) biosynthesis pathway enzyme. In some embodiments, the GPP biosynthesis pathway comprises a mevalonate (MVA) pathway, a non-mevalonate (MEP) pathway, an alternative non-MEP, non-MVA GPP pathway, or a combination thereof. In some embodiments, the GPP biosynthesis pathway enzyme is geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, or a combination thereof.

In some embodiments, the cell is a bacterial cell. In some embodiments, the cell is an E. coli cell.

In some embodiments, the disclosure provides a cell extract or cell culture medium comprising cannabigerolic acid (CBGA), cannabichromenic acid (CBCA), tetrahydrocannabinolic acid (THCA), cannabigerol (CBG), cannabichromene (CBC), cannabigerorcinic acid (CBGOA), cannabiorcichromenic acid (CBCOA), cannabigerivarinic acid (CBGVA), cannabichromevarinic acid (CBCVA), an isomer, analog or derivative thereof, or a combination thereof derived from the engineered cell described herein.

In some embodiments, the disclosure provides a method of making a cannabinoid selected from CBCA, CBC, CBCOA, CBCVA, THCA, an isomer, analog or derivative thereof, or a combination thereof, comprising: culturing the engineered cell described herein, isolating the cannabinoid from the cell extract or cell culture medium described herein, or both.

In some embodiments, the disclosure provides a method of making CBCA, THCA, or an isomer, analog or derivative thereof, comprising contacting CBGA with the non-natural flavin-dependent oxidase described herein. In some embodiments, the disclosure provides a method of making CBCA, THCA, or an isomer, analog or derivative thereof, or a combination thereof, comprising contacting CBGA with a flavin-dependent oxidase comprising any of SEQ ID NOS:1-6. In some embodiments, the flavin-dependent oxidase comprises SEQ ID NO:3.

In some embodiments, the disclosure provides a method of making CBCOA or an isomer, analog or derivative thereof, or a combination thereof, comprising contacting CBGOA with the non-natural flavin-dependent oxidase described herein. In some embodiments, the disclosure provides a method of making CBCOA or an isomer, analog or derivative thereof, or a combination thereof, comprising contacting CBGOA with a flavin-dependent oxidase of any of SEQ ID NOS:1-6. In some embodiments, the flavin-dependent oxidase comprises SEQ ID NO:3.

In some embodiments, the disclosure provides a method of making CBCVA and/or an isomer, analog or derivative thereof, or a combination thereof, comprising contacting CBGVA with the non-natural flavin-dependent oxidase described herein. In some embodiments, the disclosure provides a method of making CBCVA and/or an isomer, analog or derivative thereof, or a combination thereof, comprising contacting CBGVA with a flavin-dependent oxidase comprising any of SEQ ID NOS:1-6. In some embodiments, the flavin-dependent oxidase comprises SEQ ID NO:3.

In some embodiments, the disclosure provides a method of making CBC or an analog or derivative thereof, comprising contacting comprising contacting CBG with the non-natural flavin-dependent oxidase described herein. In some embodiments, the disclosure provides a method of making CBC or an analog or derivative thereof, comprising contacting comprising contacting CBG with a flavin-dependent oxidase comprising any of SEQ ID NOS:1-6. In some embodiments, the flavin-dependent oxidase comprises SEQ ID NO:3.

In some embodiments, the contacting occurs at about pH 4 to about pH 9. In some embodiments, the method is performed in an in vitro reaction medium. In some embodiments, the in vitro reaction medium comprises a surfactant. In some embodiments, the surfactant is about 0.01% (v/v) to about 1% (v/v) of the in vitro reaction medium. In some embodiments, the surfactant is 2-[4-(2,4,4-trimethylpentan-2-yl)phenoxy]ethanol. In some embodiments, the in vitro reaction medium comprises about 0.1% (v/v) 2-[4-(2,4,4-trimethylpentan-2-yl)phenoxy]ethanol (Triton™ X-100).

In some embodiments, the disclosure provides a method of making an isolated non-natural flavin-dependent oxidase, comprising isolating the non-natural flavin-dependent oxidase expressed in the engineered cell described herein. In some embodiments, the disclosure provides an isolated non-natural flavin-dependent oxidase made by the method described herein.

In some embodiments, the disclosure provides a composition comprising a cannabinoid or an isomer, analog or derivative thereof obtained from the engineered cell described herein, the cell extract described herein, or the method described herein. In some embodiments, the cannabinoid is CBCA, THCA, CBCOA, CBCVA, CBC, or an isomer, analog or derivative thereof, or a combination thereof. In some embodiments, the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

In some embodiments, the composition is a therapeutic or medicinal composition. In some embodiments, the composition is a topical composition. In some embodiments, the composition is an edible composition.

In some embodiments, the disclosure provides a composition comprising: (a) a flavin-dependent oxidase comprising any one of SEQ ID NOS:1-6; and (b) a cannabinoid. In some embodiments, the flavin-dependent oxidase comprises SEQ ID NO:1. In some embodiments, the flavin-dependent oxidase comprises SEQ ID NO:3. In some embodiments, the disclosure provides a composition comprising: (a) a flavin-dependent oxidase, wherein the flavin-dependent oxidase does not comprise a disulfide bond, and wherein the non-natural flavin-dependent oxidase is capable of oxidative cyclization of a prenylated aromatic compound into a cannabinoid; and (b) a cannabinoid, the prenylated aromatic compound, or both. In some embodiments, the disclosure provides a composition comprising: (a) the non-natural flavin-dependent oxidase described herein; and (b) a cannabinoid, a prenylated aromatic compound, or both. In some embodiments, the cannabinoid or prenylated aromatic compound is CBGA, CBGOA, CBGVA, CBG, CBCA, THCA, CBCOA, CBCVA, CBC, or an isomer, analog, or derivative thereof, or a combination thereof.

In some embodiments, the composition further comprises an enzyme in a cannabinoid biosynthesis pathway. In some embodiments, the cannabinoid biosynthesis pathway enzyme comprises olivetol synthase (OLS), olivetolic acid cyclase (OAC), an enzyme in a geranyl pyrophosphate (GPP) pathway, prenyltransferase, or a combination thereof.

### BRIEF DESCRIPTION OF THE DRAWINGS

The following drawings form part of the present specification and are included to further demonstrate exemplary embodiments of certain aspects of the present disclosure.

FIG. 1A shows a superimposed crystal structure of TamL (shown in green PDB ID: 273S) and Δ⁹-tetrahydrocannabinolic acid synthase (THCAS; shown in cyan; PDB ID: 3VTE). The molecules shown in ball-and-stick models are flavin adenine dinucleotide (FAD) and tirandamycin E. FIG. 1B shows a superimposed structure of EncM (shown in green; PDB ID: 3W8Z) and THCAS (shown in cyan; PDB ID: 3VTE). The molecules shown in ball-and-stick models are FAD and hydroxytetraketide ((7S)-7-hydroxy-1-phenyloctane-1,3,5-trione).

FIG. 2 is reproduced from Mantovani et al. (2013), J Am Chem Soc 135:18032-18035 and shows a predicted reaction mechanism for Clz9.

FIGS. 3A, 3B, and 3C show exemplary HPLC/MS/MS traces detecting cannabinoid products from CBGA as described herein. FIG. 3A shows the results using lysate from E. coli BL21(DE3) with empty plasmid. FIG. 3B shows the results using 105 μM purified TamL. FIG. 3C shows the results using 14.4 μM purified Cds_11170A. Reactions were conducted in 100 mM Tris-HCl, pH 7.4 with 200 μM CBGA and 0.1% Triton™ X-100. Reactions were quenched after 24 hrs at 37° C.

FIGS. 4A, 4B, and 4C show exemplary HPLC/MS/MS traces detecting cannabinoid products. FIG. 4A shows the results of the cannabinoid products from CBGA using purified EncM T139V with 0.1% Triton™ X-100. FIG. 4B shows the results of the cannabinoid products from CBGA using purified EncM T139V without 0.1% Triton™ X-100. FIG. 4C shows the results of the cannabinoid products from CBGOA using purified EncM T139V with 0.1% Triton™ X-100. Reactions were conducted in 100 mM Tris-HCl, pH 7.4 with 15 μM EncM T139V and 200 μM CBGA. Reactions were quenched after 24 hrs at 37° C.

FIGS. 5A and 5B show exemplary HPLC/MS/MS traces detecting cannabinoid products from CBGA using purified MBP-Clz9 (83 μM). FIG. 5A shows the results of experiments performed in 100 mM sodium citrate, pH 5.0, with 0.1% Triton™ X100 and 200 μM CBGA. FIG. 5B shows the results of experiments performed in 100 mM Tris-HCl, pH 7.4, with 0.1% Triton™ X100 and 200 μM CBGA. Reactions were quenched after 24 hrs incubation at 37° C.

FIG. 6A shows an exemplary ion fragmentation pattern of CBCA peak with Clz9 from CBGA substrate in LC/MS. FIG. 6B shows an exemplary ion fragmentation pattern of a CBCA authentic standard.

FIG. 7 shows a proposed reaction mechanism of Clz9 with CBGA as substrate, according to an embodiment herein.

FIGS. 8A and 8B show exemplary HPLC/MS/MS traces detecting cannabinoid products from CBGOA using purified MBP-Clz9 (83 μM). FIG. 8A shows the results of experiments performed in 100 mM Tris-HCl, pH 7.4 with 0.1% Triton™ X100 and 200 μM CBGOA. FIG. 8B shows the results of experiments performed in 100 mM sodium citrate, pH 5.0 with 0.1% Triton™ X100 and 200 μM CBGOA. Reactions were quenched after 24 hrs incubation at 37° C.

FIGS. 9A and 9B show exemplary HPLC/MS/MS traces detecting cannabinoid products from CBGVA using purified MBP-Clz9 (83 μM). FIG. 9A shows the results of experiments performed in 100 mM Tris-HCl, pH 7.4 with 0.1% Triton™ X100 and 200 μM CBGVA. FIG. 9B shows the results of experiments performed in 100 mM sodium citrate, pH 5.0 with 0.1% Triton™ X100 and 200 μM CBGVA. Reactions were quenched after 24 hrs incubation at 37° C.

FIGS. 10A and 10B show exemplary HPLC/MS/MS traces detecting cannabinoid products from CBG using purified MBP-Clz9 (83 μM). FIG. 10A shows the results of experiments performed in 100 mM Tris-HCl, pH 7.4 with 0.1% Triton™ X100 and 167 μM CBG. FIG. 10B shows the results of experiments performed in 100 mM sodium citrate, pH 5.0 with 0.1% Triton™ X100 and 167 μM CBG. Reactions were quenched after 24 hrs at 37° C.

FIG. 11 shows the structure of cannabigerolic acid (CBGA), cannabigerivarinic acid (CBGVA), cannabigerorcinic acid (CBGOA), cannabichromenic acid (CBCA), cannabichromevarinic acid (CBCVA), and cannabiorcichromenic acid (CBCOA).

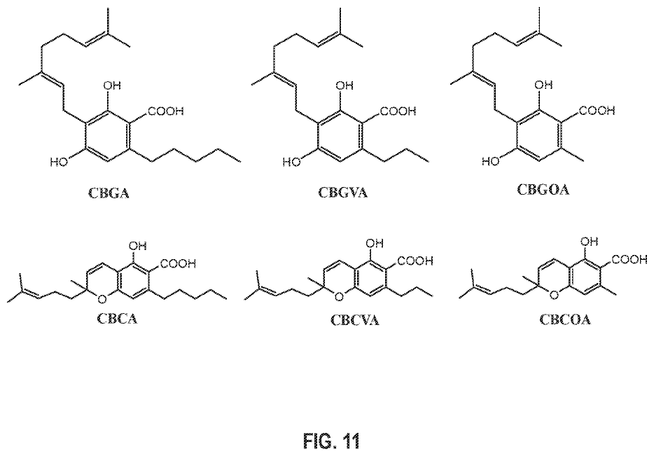

FIG. 12 shows a proposed reaction mechanism of Clz9 with CBG as substrate, according to an embodiment herein.

FIGS. 13A-13D show exemplary LC/MS spectra detecting cannabinoid products from CBGA using wild type or mutant Clz9. FIG. 13A shows the CBGA conversion product profile wild type Clz9. FIG. 13B shows the CBGA conversion product profile of Clz9 H402A variant. FIG. 13C shows the CBGA conversion product profile of Clz9 N400W variant. FIG. 13D shows the CBGA conversion product profile of Clz9 T438Y variant. All reaction spectra were monitored by LC/MS at 357/339 MRM transition. The product at 0.46 mins is an unknown “CBCA-like” cannabinoid as described herein. The product at 0.8 mins is CBCA.

FIG. 14 shows the in vitro CBCA synthase activity of N-terminally truncated Clz9 variants as compared to full-length Clz9. Purified proteins were analyzed as described in embodiments herein. Sequences of the N-terminally truncated Clz9 variants are shown below the graph. FIG. 14 discloses SEQ ID NOS 22-25, respectively, in order of appearance.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y, and any numbers that fall within x and y.

A “nucleic acid,” “nucleic acid molecule,” “nucleic acid sequence,” “nucleotide sequence,” “oligonucleotide,” or “polynucleotide” means a polymeric compound including covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a nucleic acid encoding any one of the polypeptides disclosed herein, e.g., is directed to a polynucleotide encoding a flavin-dependent oxidase or a variant thereof.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “operably linked” means that a polynucleotide of interest, e.g., the polynucleotide encoding a nuclease, is linked to the regulatory element in a manner that allows for expression of the polynucleotide. In some embodiments, the regulatory element is a promoter. In some embodiments, a nucleic acid expressing the polypeptide of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the various vectors of the present disclosure.

An “expression vector” or vectors (“an expression construct”) can be constructed to include one or more protein of interest-encoding nucleic acids (e.g., nucleic acid encoding a THCAS described herein) operably linked to expression control sequences functional in the host organism. Expression vectors applicable for use in the microbial host organisms provided include, for example, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). In some embodiments, the expression vector comprises a nucleic acid encoding a protein described herein, e.g., a flavin-dependent oxidase.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like. When two or more exogenous encoding nucleic acids (e.g., a gene encoding a flavin-dependent oxidase and an additional gene encoding another enzyme in a cannabinoid biosynthesis pathway such as, e.g., OLS, OAC, prenyltransferase, and/or an enzyme in the GPP pathway as described herein) are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.” In some embodiments, the present disclosure provides a host cell comprising an expression vector that comprises a nucleic acid encoding a flavin-dependent oxidase or variant thereof. In some embodiments, the host cell is a bacterial cell, a fungal cell, an algal cell, a cyanobacterial cell, or a plant cell.

A genetic alteration that makes an organism or cell non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

A host cell, organism, or microorganism engineered to express or overexpress a gene, a nucleic acid, nucleic acid sequence, or nucleic acid molecule, or to overexpress an enzyme or polypeptide has been genetically engineered through recombinant DNA technology to include a gene or nucleic acid sequence that it does not naturally include that encodes the enzyme or polypeptide or to express an endogenous gene at a level that exceeds its level of expression in a non-altered cell. As non-limiting examples, a host cell, organism, or microorganism engineered to express or overexpress a gene, a nucleic acid, nucleic acid sequence, or nucleic acid molecule, or to overexpress an enzyme or polypeptide can have any modifications that affect a coding sequence of a gene, the position of a gene on a chromosome or episome, or regulatory elements associated with a gene. A gene can also be overexpressed by increasing the copy number of a gene in the cell or organism. In some embodiments, overexpression of an endogenous gene comprises replacing the native promoter of the gene with a constitutive promoter that increases expression of the gene relative to expression in a control cell with the native promoter. In some embodiments, the constitutive promoter is heterologous.

Similarly, a host cell, organism, or microorganism engineered to under-express (or to have reduced expression of) a gene, nucleic acid, nucleic acid sequence, or nucleic acid molecule, or to under-express an enzyme or polypeptide can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express or down-regulate a gene also include modifications to regulatory regions of the gene that can reduce its expression.

The term “exogenous” is intended to mean that the referenced molecule or the referenced activity is introduced into the host cell or host organism. The molecule can be introduced, for example, by introduction of an encoding nucleic acid into the host genetic material such as by integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a heterologous nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

Accordingly, the term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host microbial organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or a combination thereof, and still be considered as more than one exogenous nucleic acid. For example, as disclosed herein, a host cell or host organism can be engineered to express at least two, three, four, five, six, seven, eight, nine, ten or more exogenous nucleic acids encoding a desired pathway enzyme or protein. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, can be integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell host cell or host organism using techniques well known in the art including, but not limited to, conjugation, electroporation, chemical transformation, transduction, transfection, and ultrasound transformation. Optionally, for exogenous expression in E. coli or other prokaryotic host cells, some nucleic acid sequences in the genes or cDNAs of eukaryotic nucleic acids can encode targeting signals such as an N-terminal mitochondrial or other targeting signal, which can be removed before transformation into the prokaryotic host cells, if desired. For example, removal of a mitochondrial leader sequence led to increased expression in E. coli (Hoffmeister et al. (2005), J Biol Chem 280: 4329-4338). For exogenous expression in yeast or other eukaryotic host cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon optimized polynucleotides expressing a flavin-dependent oxidase or variant thereof.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide. Unless otherwise specified, sequences of polypeptides throughout the present disclosure are listed from N-terminus to C-terminus, and sequences of polynucleotides throughout the present disclosure are listed from the 5′ end to the 3′ end.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photo-cross-linked moieties, uniquely reactive functional groups, and NMR, IR, and x-ray crystallographic probes. Exemplary unnatural or synthetic amino acids are provided in, e.g., Mitra et al. (2013), Mater Methods 3:204 and Wals et al. (2014), Front Chem 2:15. Unnatural amino acids may also include naturally-occurring compounds that are not typically incorporated into a protein or polypeptide, such as, e.g., citrulline (Cit), selenocysteine (Sec), and pyrrolysine (Pyl).

As used herein, the terms “non-natural,” “non-naturally occurring,” “variant,” and “mutant” are used interchangeably in the context of an organism, polypeptide, or nucleic acid. The terms “non-natural,” “non-naturally occurring,” “variant,” and “mutant” in this context refer to a polypeptide or nucleic acid sequence having at least one variation or mutation at an amino acid position or nucleic acid position as compared to a wild-type polypeptide or nucleic acid sequence. The at least one variation can be, e.g., an insertion of one or more amino acids or nucleotides, a deletion of one or more amino acids or nucleotides, or a substitution of one or more amino acids or nucleotides. A “variant” protein or polypeptide is also referred to as a “non-natural” protein or polypeptide.

Naturally-occurring organisms, nucleic acids, and polypeptides can be referred to as “wild-type,” “wild type” or “original” or “natural” such as wild type strains of the referenced species, or a wild-type protein or nucleic acid sequence. Likewise, amino acids found in polypeptides of the wild type organism can be referred to as “original” or “natural” with regards to any amino acid position.

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5th) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

As used herein, the term “sequence similarity” (% similarity) refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: Positively-charged side chains: Arg, His, Lys; Negatively-charged side chains: Asp, Glu; Polar, uncharged side chains: Ser, Thr, Asn, Gln; Hydrophobic side chains: Ala, Val, Ile, Leu, Met, Phe, Tyr, Trp; Other: Cys, Gly, Pro.

In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

The “percent identity” (% identity) between two polynucleotide or polypeptide sequences is determined when sequences are aligned for maximum homology, and generally not including gaps or truncations. Additional sequences added to a polypeptide sequence, such as but not limited to immunodetection tags, purification tags, localization sequences (presence or absence), etc., do not affect the % identity.

Algorithms known to those skilled in the art, such as Align, BLAST, ClustalW and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest.

In some embodiments, similar polynucleotides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical nucleic acid sequence. In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acid sequence.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least at least 80%, at least 85%, at least 90%, at least 95%, at least 96%, at least 97% at least 98%, or at least 99% nucleotide or amino acid identity, respectively, to a reference sequence.

As used in the context of proteins, the term “structural similarity” indicates the degree of homology between the overall shape, fold, and/or topology of the proteins. It should be understood that two proteins do not necessarily need to have high sequence similarity to achieve structural similarity. Protein structural similarity is often measured by root mean squared deviation (RMSD), global distance test score (GDT-score), and template modeling score (TM-score); see, e.g., Xu and Zhang (2010), Bioinformatics 26(7):889-895. Structural similarity can be determined, e.g., by superimposing protein structures obtained from, e.g., x-ray crystallography, NMR spectroscopy, cryogenic electron microscopy (cryo-EM), mass spectrometry, or any combination thereof, and calculating the RMSD, GDT-score, and/or TM-score based on the superimposed structures. In some embodiments, two proteins have substantially similar tertiary structures when the TM-score is greater than about 0.5, greater than about 0.6, greater than about 0.7, greater than about 0.8, or greater than about 0.9. In some embodiments, two proteins have substantially identical tertiary structures when the TM-score is about 1.0. Structurally-similar proteins may also be identified computationally using algorithms such as, e.g., TM-align (Zhang and Skolnick, Nucleic Acids Res 33(7):2302-2309, 2005); DALI (Holm and Sander, J Mol Biol 233(1):123-138, 1993); STRUCTAL (Gerstein and Levitt, Proc Int Conf Intell Syst Mol Biol 4:59-69, 1996); MINRMS (Jewett et al., Bioinformatics 19(5):625-634, 2003); Combinatorial Extension (CE) (Shindyalov and Bourne, Protein Eng 11(9):739-747, 1998); ProtDex (Aung et al., DASFAA 2003, Proceedings); VAST (Gibrat et al., Curr Opin Struct Biol 6:377-385, 1996); LOCK (Singh and Brutlag, Proc Int Conf Intell Syst Mol Biol 5:284-293, 1997); SSM (Krissinel and Henrick, Acta Cryst D60:2256-2268, 2004), and the like.

### Flavin-Dependent Oxidase

Cannabinoid synthases are enzymes responsible for the biosynthesis of cannabinoids, e.g., cannabinoid compounds described herein. The only naturally-occurring cannabinoid synthase enzymes currently known to convert cannabigerolic acid (CBGA) or its analogs to cannabinoids such as Δ9-tetrahydrocannabinolic acid (THCA) by THCA synthase (THCAS, EC 1.21.3.7), cannabidiolic acid (CBDA) by CBDA synthase (CBDAS, EC 1.21.3.8) or cannabichromenic acid (CBCA) by CBCA synthase (CBCAS) or their analogs are from the plant Cannabis sativa (Onofri et al. (2015), J Mol Biol 423:96; Laverty et al. (2019), Genome Research 29:146-156). It is challenging to utilize these enzymes from C. sativa for heterologous cannabinoid production in microorganisms such as bacteria because they are typically secreted proteins that require a disulfide bond and glycosylation, are poorly active, and require low pH for optimal activity (Zirpel et al. (2018), J Biotechnol 284:17-26). Thus, cannabinoid synthase enzymes from C. sativa are not conducive for standard microbial fermentation processes.

The present inventors have discovered and engineered alternative enzymes for the improved microbial production of cannabinoids. The enzymes described herein do not contain a disulfide bond, do not require glycosylation, and are active at neutral pH. Thus, these enzymes are suitable for soluble and active expression in a microbial host under standard fermentation conditions. In some embodiments, the enzyme is a bacterial or fungal enzyme.

In some embodiments, the disclosure provides a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid.

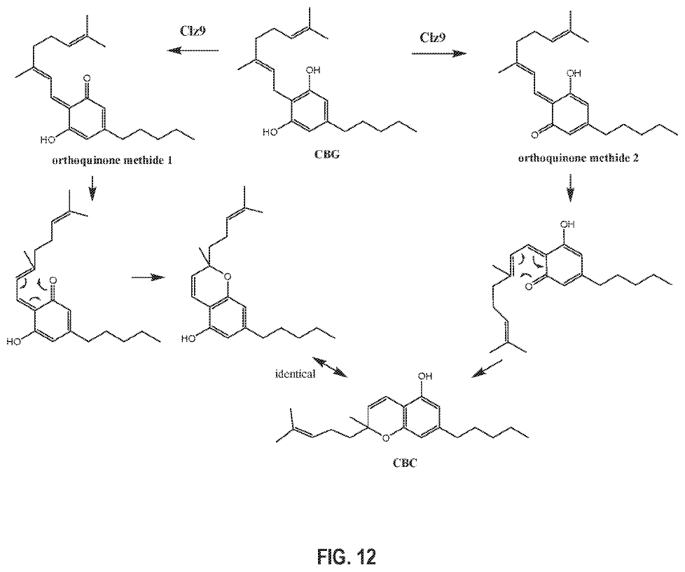

As used herein, “cannabinoid” refers to a prenylated polyketide or terpenophenolic compound derived from fatty acid or isoprenoid precursors. In general, cannabinoids are produced via a multi-step biosynthesis pathway, with the final precursor being a prenylated aromatic compound. In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG). In some embodiments, the prenylated aromatic compound is converted into a cannabinoid by oxidative cyclization. An exemplary oxidative cyclization reaction from CBG to an orthoquinone methide (oxidation) then to CBC (cyclization) is shown in FIG. 12. In some embodiments, the non-natural flavin-dependent oxidase converts one or more of CBGA, CBGOA, CBGVA, and CBG into a cannabinoid. In some embodiments, the non-natural flavin-dependent oxidase converts CBGA into one or more of CBCA, CBDA, or THCA. In some embodiments, the non-natural flavin-dependent oxidase converts CBGOA into one or more of CBCOA, CBDOA, or THCOA. In some embodiments, the non-natural flavin-dependent oxidase converts CBGVA into one or more of CBCVA, CBDVA, or THCVA. In some embodiments, the non-natural flavin-dependent oxidase converts CBGO into one or more of CBCO, CBDO, or THCO. In some embodiments, the non-natural flavin-dependent oxidase converts CBGO into CBCO. In some embodiments, the non-natural flavin-dependent oxidase converts CBGV into one or more of CBCV, CBDV, or THCV. In some embodiments, the non-natural flavin-dependent oxidase converts CBGV into CBCV. In some embodiments, the non-natural flavin-dependent oxidase converts CBG into one or more of CBC, CBD, or THC.

Different cannabinoids can be produced based on the way that a precursor is cyclized. For example, THCA, CBDA, and CBCA are produced by oxidative cyclization of CBGA. Further examples of cannabinoids include, but are not limited to, THCA, THCV, THCO, THCVA, THCOA, THC, CBDA, CBDV, CBDO, CBDVA, CBDOA, CBD, CBCA, CBCV, CBCO, CBCVA, CBCOA, CBC, cannabinolic acid (CBNA), cannabinol (CBN), cannabicyclol (CBL), cannabivarin (CBV), cannabielsoin (CBE), cannabicitran, and isomers, analogs or derivatives thereof. As used herein, an “isomer” of a reference compound has the same molecular formula as the reference compound, but with a different arrangement of the atoms in the molecule. As used herein, an “analog” or “structural analog” of a reference compound has a similar structure as the reference compound, but differs in a certain component such as an atom, a functional group, or a substructure. An analog can be imagined to be formed from the reference compound, but not necessarily formed or derived from the reference compound. As used herein, a “derivative” of a reference compound is derived from a similar compound by a similar reaction. Methods of identifying isomers, analogs or derivatives of the cannabinoids described herein are known to one of ordinary skill in the art.

In some embodiments, the non-natural flavin-dependent oxidase is a berberine bridge enzyme (BBE)-like enzyme. BBE-like enzymes are described, e.g., in Daniel et al. (2017), Arch Biochem Biophys 632:88-103 and include protein family domains (Pfams) PF08031 (berberine-bridge domain) and PF01564 (flavin adenine dinucleotide (FAD)-binding domain). In general, a BBE-like enzyme comprises a FAD binding module that is formed by the N- and C-terminal portions of the protein, and a central substrate binding domain that, together with the FAD cofactor, provides the environment for efficient substrate binding, oxidation and cyclization. A non-limiting list of BBE-like enzymes and are described in Table 1. It will be understood by one of ordinary skill in the art that, in some embodiments, a BBE-like enzyme binds a flavin mononucleotide (FMN) in addition to or instead of FAD.

TABLE 1
Exemplary BBE-like enzymes with published structures.

| Protein | Organism | PDB Entries | Cofactor(s), ligand(s) |
|---|---|---|---|
| Reticuline | Eschscholzia | 3D2D, 3D2H, 3D2J, | FAD, (S)-reticuline, (S)- |
| Oxidase/ | californica | 3FW7, 3WF8, 3WF9, | scoulerine, dehydroscoulerine |
| Dehydrogenase | | 3WFA, 3GSY, 4EC3, | |
| | | 4PZF | |
| Pollen Allergen | Phleum pratense | 3TSH, 3TSJ, 4PVE, | FAD |
| PhI p 4.0202 | | 4PVH, 4PVK, 4PVJ, | |
| | | 4PWB, 4PWC, | |
| MaDA | Morus alba | 6JQH | FAD |
| THCA Synthase | Cannabis sativa | 3VTE | FAD |
| Pyrimidine | Escherichia coli | 6SGG, 6SGL, 6SGM, | FMN, 2,4-dimethoxypyrimidine |
| monooxygenase | K-12 | 6SGN, 6TEE, 6TEF, | |
| RutA | | 6TEG | |
| Caerulomycin | Actinoalloteichus | 5I1V, 5I1W | FAD, 6-(hydroxymethyl)[2,2′- |
| Oxidase K | sp. WH1-2216-6 | | bipyridin]-4-ol, 4-hydroxy[2,2′- |
| (CrmK) | | | bipyridine]-6-carbaldehyde |
| Tirandamycin | Streptomyces sp. | 2Y08, 2Y3R, 2Y3S, | FAD, Tirandamycin D, |
| Oxidase L | 307-9 | 2Y4G | Tirandamycin E |
| (TamL) | | | |
| EncM | Streptomyces | 3W8W, 3W8X, 3W8Z, | FAD, 6,6,6-trifluoro-1- |
| | maritimus | 4XLO, 6FOQ, 6FOW, | phenylhexane-1,3,5-trione, (7S)- |
| | | 6FP3, 6FY8, 6FY9, | 7-hydroxy-1-phenyloctane-1,3,5- |
| | | 6FYA, 6FYB, 6FYC, | trione |
| | | 6FYD, 6FYE, 6FYF, | |
| | | 6FYG | |

In some embodiments, the non-natural flavin-dependent oxidase has substantial structural similarity with a cannabinoid synthase from C. sativa, e.g., Δ9-tetrahydrocannabinolic acid synthase (THCAS). THCAS utilizes a FAD cofactor when catalyzing the conversion of substrate CBGA to THCA. In some embodiments, the enzyme comprises a structurally similar active site as a cannabinoid synthase from C. sativa, e.g., THCAS. As used herein, the term “active site” refers to one or more regions in an enzyme that may be important for catalysis, substrate binding, and/or cofactor binding.

In some embodiments, the non-natural flavin-dependent oxidase has at least 30% sequence identity to SEQ ID NO:1, 3, 19, or 20. In some embodiments, the non-natural flavin-dependent oxidase has at least 40% sequence identity to SEQ ID NO:1, 3, 19, or 20. In some embodiments, the non-natural flavin-dependent oxidase has at least 70% sequence identity to SEQ ID NO:1, 3, 19, or 20. In some embodiments, the non-natural flavin-dependent oxidase has at least 50% sequence identity to SEQ ID NO:1, 3, 19, or 20. In some embodiments, the non-natural flavin-dependent oxidase has at least 60% sequence identity to SEQ ID NO:1, 3, 19, or 20. In some embodiments, the non-natural flavin-dependent oxidase has at least 70% sequence identity to SEQ ID NO:1, 3, 19, or 20. In some embodiments, the non-natural flavin-dependent oxidase has at least 80% sequence identity to SEQ ID NO:1, 3, 19, or 20. In some embodiments, the non-natural flavin-dependent oxidase has at least 85% sequence identity to SEQ ID NO:1, 3, 19, or 20. In some embodiments, the non-natural flavin-dependent oxidase has at least 90% sequence identity to SEQ ID NO:1, 3, 19, or 20. In some embodiments, the non-natural flavin-dependent oxidase has at least 95% sequence identity to SEQ ID NO:1, 3, 19, or 20.

As described herein, a “non-natural” protein or polypeptide refers to a protein or polypeptide sequence having at least one variation at an amino acid position as compared to a wild-type polypeptide sequence. In some embodiments, the non-natural flavin-dependent oxidase has at least one variation at an amino acid position as compared to a wild-type flavin-dependent oxidase.

In some embodiments, the at least one amino acid variation comprises a substitution, deletion, insertion, or a combination thereof. In some embodiments, the variation comprises an amino acid substitution. In some embodiments, the variation comprises a deletion of one or more amino acids, e.g., about 1 to about 100, about 2 to about 80, about 5 to about 50, about 10 to about 40, about 12 to about 35, or about 14 to about 30 amino acids. In some embodiments, the variation comprises an insertion of one or more amino acids. In some embodiments, the at least one amino acid variation in the non-natural flavin-dependent oxidase is not in an active site of the flavin-dependent oxidase. In some embodiments, the active site of the flavin-dependent oxidase comprises one or more amino acid residues involved in binding the substrate, e.g., CBGA, CBGOA, CBGVA, CBGO, CBGV, and/or CBG. In some embodiments, the active site of the flavin-dependent oxidase comprises one or more amino acid residues involved in binding FAD cofactor. In some embodiments, the active site of the flavin-dependent oxidase comprises one or more amino acid residues involved for catalysis, e.g., the oxidative cyclization of CBGA into CBCA.

In some embodiments, the non-natural flavin-dependent oxidase does not comprise a disulfide bond. In the context of a protein or polypeptide, a disulfide bond (sometimes called a “S—S bond” or “disulfide bridge”) refers to a covalent bond between two cysteine residues, typically formed through oxidation of the thiol groups on the cysteines. Proteins comprising disulfide bonds, e.g., endogenous to plants, can be unstable in bacterial host cells as the disulfide bonds are often disrupted due to the reducing environment in bacterial cells. In some embodiments, cannabinoid synthases from C. sativa are substantially unstable in a bacterial cell, e.g., an E. coli cell. As used herein, “unstable” protein can refer to proteins that are non-functional, denatured, and/or degraded rapidly, resulting in catalytic activity that is greatly reduced relative to the activity found in its native host cell, e.g., C. sativa plants. In some embodiments, the lack of a disulfide bond in the non-natural flavin-dependent oxidase advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more of the non-natural flavin-dependent oxidase that does not comprise a disulfide bond as compared with a flavin-dependent oxidase that comprises a disulfide bond, e.g., a wild-type cannabinoid synthase from C. sativa.

In some embodiments, the non-natural flavin-dependent oxidase is not glycosylated. As used herein, glycosylation refers to the addition of one or more sugar molecules to another biomolecule, e.g., a protein or polypeptide. Glycosylation can play an important role in the folding, secretion, and stability of proteins (see, e.g., Drickamer and Taylor, Introduction to Glycobiology (2^{nd} ed.), Oxford University Press, USA). Glycosylation mechanisms and patterns in bacteria and eukaryotes are distinct from one another. Moreover, the most common type of glycosylation, N-linked glycosylation, occurs in eukaryotes but not in bacteria. Thus, bacterial cells are generally not suitable for the production of eukaryotic proteins that are glycosylated, e.g., the cannabinoid synthases from C. sativa. In some embodiments, the lack of glycosylation in the non-natural flavin-dependent oxidase further advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more (e.g., by weight) of the non-natural flavin-dependent oxidase that is not glycosylated, compared with a flavin-dependent oxidase that is glycosylated, e.g., a wild-type cannabinoid synthase from C. sativa.

In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more of the non-natural flavin-dependent oxidase that does not comprise a disulfide bond and is not glycosylated, compared with a flavin-dependent oxidase that comprises a disulfide bond and is glycosylated, e.g., a wild-type cannabinoid synthase from C. sativa.

In some embodiments, the non-natural flavin-dependent oxidase utilizes a flavin cofactor, e.g., FAD or FMN, for catalytic activity. In some embodiments, the non-natural flavin-dependent oxidase utilizes a FAD cofactor for catalytic activity, e.g., the conversion of CBGA, CBGOA, CBGVA, and/or CBG into a cannabinoid. In some embodiments, the non-natural dependent oxidase comprises a monovalently bound FAD cofactor. As used herein, “monovalently bound” means that the FAD is covalently bound to one amino acid residue of the protein, e.g., the non-natural flavin-dependent oxidase. In some embodiments, the non-natural flavin-dependent oxidase comprises a bivalently bound FAD cofactor. As used herein, “bivalently bound” means that the FAD is covalently bound to two amino acid residues of the protein, e.g., the non-natural flavin-dependent oxidase. In some embodiments, the cannabinoid synthases from C. sativa comprise bivalently bound FAD cofactor. In some embodiments, the FAD cofactor is covalently bound to a histidine and/or a cysteine of the non-natural flavin-dependent oxidase.

In some embodiments, catalytic activity of the non-natural flavin-dependent oxidase is substantially the same from about pH 4 to about pH 9. In some embodiments, catalytic activity of the non-natural flavin-dependent oxidase is substantially the same from about pH 4.5 to about pH 8.5. In some embodiments, catalytic activity of the non-natural flavin-dependent oxidase is substantially the same from about pH 5 to about pH 8. In some embodiments, catalytic activity of the non-natural flavin-dependent oxidase is substantially the same from about pH 5.5 to about pH 7.5. In some embodiments, catalytic activity of the non-natural flavin-dependent oxidase is substantially the same from about pH 5 to about pH 7. In some embodiments, catalytic activity of the non-natural flavin-dependent oxidase is substantially the same at about pH 5 and at about pH 7. The term “substantially” when referring to enzyme activity at different pH conditions means that the non-natural flavin-dependent oxidase enzyme activity does not vary (increase or decrease) by more than 20%, more than 15%, more than 10%, more than 5%, or more than 1% under the different pH conditions. In some embodiments, catalytic activity of the non-natural flavin-dependent oxidase does not vary more than 20%, more than 15%, more than 10%, more than 5%, or more than 1% from about pH 5 to about pH 8. As described herein, cannabinoid synthases from C. sativa generally require low pH (around 5 to 5.5) for optimal activity and are less active at neutral pH (see, e.g., Zirpel et al. (2018), J Biotechnol 284:17-26). The catalytic activity of the non-natural flavin-dependent oxidase does not vary substantially over a wide range of pH (e.g., from about pH 5 to about pH 8), which is beneficial for microbial production of cannabinoids.

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a natural, i.e., wild-type, flavin-dependent oxidase. As described herein, the terms “natural” or “wild-type” flavin-dependent oxidase can refer to any known flavin-dependent oxidase sequence. For example, a natural flavin-dependent oxidase can include, but is not limited to, EncM from Streptomyces maritimus (see, e.g., Teufel et al. (2013), Nature 503:552-556), Clz9 from Streptomyces sp. CNH-287 (see, e.g., Mantovani et al. (2013), J Am Chem Soc 135:18032-18035), and the proteins listed in Table 1.

In some embodiments, the disclosure provides a non-natural flavin-dependent oxidase with about 70%, 75%, 80%, 85%, 90%, 95%, 99% or greater identity to at least about 25, 50, 75, 100, 125, 150, 200, 250, 300, 350, 400, 450, 500, or more contiguous amino acids of SEQ ID NO:1 or 3, comprising at least one amino acid variation as compared to a wild type flavin-dependent oxidase, wherein the non-natural flavin-dependent oxidase converts one or more of cannabigerolic acid (CBGA), cannabigerorcinic acid (CBGOA), cannabigerivarinic acid (CBGVA), and cannabigerol (CBG) into a cannabinoid. In some embodiments, the disclosure provides a non-natural flavin-dependent oxidase with about 70%, 75%, 80%, 85%, 90%, 95%, 99% or greater identity to at least about 25, 50, 75, 100, 125, 150, 200, 250, or 300 or more contiguous amino acids of SEQ ID NO:19 or 20, wherein the non-natural flavin-dependent oxidase converts one or more of cannabigerolic acid (CBGA), cannabigerorcinic acid (CBGOA), cannabigerivarinic acid (CBGVA), and cannabigerol (CBG) into a cannabinoid.

The flavin-dependent oxidases provided herein surprisingly converted CBG into cannabichromene (CBC). Cannabinoid synthases from C. sativa are not known to accept cannabigerol (CBG) as a substrate. Thus, the flavin-dependent oxidases described herein provide the additional benefit of expanding the repertoire of cannabinoids that can be produced enzymatically by microbial host cells, e.g., bacterial cells.

### EncM

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:1. SEQ ID NO:1 describes the amino acid sequence of the EncM protein from Streptomyces maritimus.

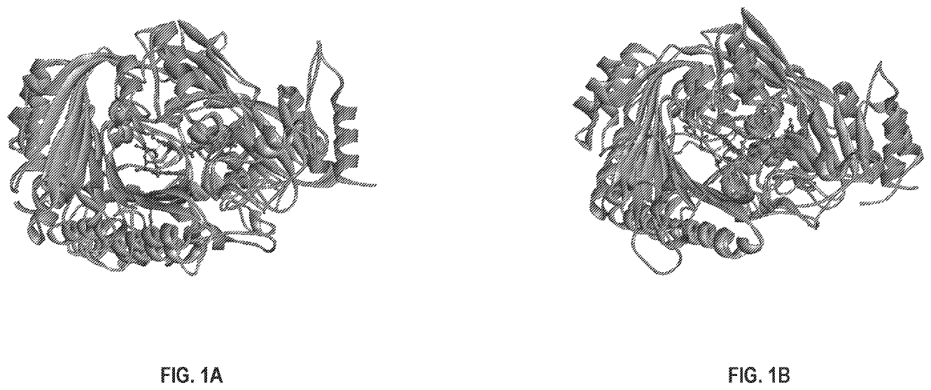

The present inventors found that EncM from Streptomyces maritimus shared structural similarity with THCAS. Unless otherwise specified, “THCAS,” “CBDAS,” and “CBCAS” as used herein refer to the wild-type THCAS from C. sativa, the wild-type CBDAS from C. sativa, and the wild-type CBCAS from C. sativa, respectively. “EncM” as used herein refers to the wild-type EncM from Streptomyces maritimus. EncM contains two N-terminal alpha-helices in a similar manner as THCAS, but unlike THCAS, the alpha-helices in EncM are not stabilized by a disulfide bond. In further contrast to THCAS, which bivalently binds the FAD cofactor, wild-type EncM binds FAD monovalently. However, upon structural superimposition, the inventors noticed that the EncM substrate-binding site is similar to that of THCAS. See FIG. 1B.

THCAS binds FAD via amino acid residues His114 and Cys176. Sequence alignment of various enzymes in the BBE family showed that the amino acid residues surrounding the corresponding His and Cys residues are generally highly conserved. EncM contains the His residue (His78; amino acid numbering with respect to SEQ ID NO:1) but contains a Val residue (Val136) at the place of the required Cys residue for bivalent attachment of FAD. The characteristic motifs for bivalent attachment of the His and Cys residues to the 8α- and 6-positions of FAD are R/KxxGH and CxxV/L/IG (see, e.g., Daniel et al. (2017), Arch Biochem Biophys 632:88-103). EncM also does not contain a highly conserved Val/Leu/Ile residue in the second motif, which should appear at position 139, and instead has a Thr residue (Thr139). Further amino acid residues have been shown to play a role in the bivalent attachment of FAD; see, e.g., Kopacz et al. (2014), Bioorg Med Chem 20:5621-5627.

Structural similarity between two proteins does not necessarily mean that they will share functional similarity. For example, TamL from Streptomyces sp. 307-9 is also structurally similar to THCAS (see FIG. 1A), but TamL did not exhibit any cannabinoid synthase activity when provided with a variety of cannabinoid precursors as substrate. Thus, while EncM and THCAS share structural similarity, it was nevertheless surprising that EncM showed cannabinoid synthase activity.

In some embodiments, the non-natural flavin-dependent oxidase having at least 70%, at least 80%, at least 85%, at least 90% or at least 95% sequence identity to SEQ ID NO:1 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type EncM. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type EncM. In some embodiments, the amino acid variation in the non-natural flavin-dependent oxidase is an amino acid substitution, deletion, or insertion. In some embodiments, the variation is a substitution of one or more amino acids in the wild-type EncM polypeptide sequence. In some embodiments, the variation is a deletion of one or more amino acids in the wild-type EncM polypeptide sequence. In some embodiments, the variation is an insertion of one or more amino acids in the wild-type EncM polypeptide sequence.

In some embodiments, the non-natural flavin-dependent oxidase having at least 70%, at least 80%, at least 85%, at least 90% or at least 95% sequence identity to SEQ ID NO:1 comprises a variation at amino acid position V136, S137, T139, L144, Y249, F313, Q353, or a combination thereof, wherein the amino acid position corresponds to SEQ ID NO:1. In some embodiments, the variation is in an active site of wild-type EncM. In some embodiments, the variation is in a FAD-binding site of wild-type EncM. In some embodiments, the variation is a substitution of the amino acid residue in wild-type EncM with the corresponding amino acid residue in the active site of a wild-type cannabinoid synthase from C. sativa, e.g., THCAS, CBDAS, or CBCAS as described herein.

In some embodiments, the variation in the non-natural flavin-dependent oxidase having at least 70%, at least 80%, at least 85%, at least 90% or at least 95% sequence identity to SEQ ID NO:1 comprises an amino acid substitution selected from V136C, S137P, T139V, L144H, Y249H, F313A, Q353N, and a combination thereof, wherein the amino acid position corresponds to SEQ ID NO:1. In some embodiments, the variation comprises a substitution at T139. In some embodiments, the variation comprises substitutions at V136 and T139. In some embodiments, the variation comprises substitutions at V136, T139, and L144. In some embodiments, the variation comprises substitutions at V136, S137, and T139. In some embodiments, the variation comprises substitutions at V136, S137, T139, and Y249. In some embodiments, the variation comprises substitutions at V136, S137, T139, Y249, and F313. In some embodiments, the variation comprises substitutions at V136, S137, T139, Y249, and Q353. In some embodiments, the variation comprises substitutions at V136, S137, T139, Y249, F313, and Q353. In some embodiments, the variation comprises T139V. In some embodiments, the variation comprises V136C T139V. In some embodiments, the variation comprises V136C T139V L144H. In some embodiments, the variation comprises V136C S137P T139V. In some embodiments, the variation comprises V136C S137P T139V Y249H. In some embodiments, the variation comprises V136C S137P T139V Y249H F313A. In some embodiments, the variation comprises V136C S137P T139V Y249H Q353N. In some embodiments, the variation comprises V136C S137P T139V Y249H F313 Q353N.

In some embodiments, the non-natural flavin-dependent oxidase having at least 70%, at least 80%, at least 85%, at least 90% or at least 95% sequence identity to SEQ ID NO:1 has a substantially similar tertiary structure as a wild-type cannabinoid synthase, e.g., THCAS, CBDAS, or CBCAS as described herein. In some embodiments, the non-natural flavin-dependent oxidase has a substantially similar active site structure as a wild-type cannabinoid synthase, e.g., THCAS, CBDAS, or CBCAS as described herein. In some embodiments, the non-natural flavin-dependent oxidase has a substantially similar FAD cofactor binding site structure as a wild-type cannabinoid synthase, e.g., THCAS, CBDAS, or CBCAS as described herein.

In some embodiments, the non-natural flavin-dependent oxidase having at least 70%, at least 80%, at least 85%, at least 90% or at least 95% sequence identity to SEQ ID NO:1 is capable of converting CBGA to cannabichromenic acid (CBCA), tetrahydrocannabinolic acid (THCA), cannabidiolic acid (CBDA), or a combination hereof. In some embodiments, the non-natural flavin-dependent oxidase converts CBGA to CBDA. In some embodiments, the non-natural flavin-dependent oxidase converts CBGA to THCA. In some embodiments, the non-natural flavin-dependent oxidase converts CBGA to CBCA. In some embodiments, the non-natural flavin-dependent oxidase has at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least or about 99%, or at least about 100% of the catalytic activity of a wild-type CBCAS. As referred to throughout the application, when comparing the catalytic activity of at least two enzymes, it will be understood by one of ordinary skill in the art that the enzymes can be subjected to the same or substantially the same reaction conditions or the enzymes can be subjected to the optimal reaction conditions for each enzyme, and catalytic activity is assessed using the same or substantially the same methods and/or equipment. Optimal reaction conditions for the enzymes described herein can be determined by one of ordinary skill in the art.

In some embodiments, the non-natural flavin-dependent oxidase having at least 70%, at least 80%, at least 85%, at least 90% or at least 95% sequence identity to SEQ ID NO:1 converts CBGA to CBCA at about pH 4 to about pH 9. In some embodiments, the non-natural flavin-dependent oxidase converts CBGA to CBCA at about pH 4.5 to about pH 8.5. In some embodiments, the non-natural flavin-dependent oxidase converts CBGA to CBCA at about pH 5 to about pH 8. In some embodiments, the non-natural flavin-dependent oxidase converts CBGA to CBCA at about pH 5.5 to about pH 7.5. In some embodiments, the non-natural flavin-dependent oxidase converts CBGA to CBCA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGA to CBCA at about pH 5. In some embodiments, the non-natural flavin-dependent oxidase converts CBGA to CBCA at about pH 7.4 or about pH 7.5.

In some embodiments, the non-natural flavin-dependent oxidase having at least 70%, at least 80%, at least 85%, at least 90% or at least 95% sequence identity to SEQ ID NO:1 is further capable of converting CBGOA to cannabiorcichromenic acid (CBCOA), cannabidiorsellinic acid (CBDOA), tetraydrocannabiorcolic acid (THCOA), or a combination thereof. In some embodiments, the non-natural flavin-dependent oxidase converts CBGOA to CBDOA. In some embodiments, the non-natural flavin-dependent oxidase converts CBGOA to THCOA. In some embodiments, the non-natural flavin-dependent oxidase converts CBGOA to CBCOA. In some embodiments, the non-natural flavin-dependent oxidase converts CBGOA to CBCOA at about pH 4 to about pH 9. In some embodiments, the non-natural flavin-dependent oxidase converts CBGOA to CBCOA at about pH 4.5 to about pH 8.5. In some embodiments, the non-natural flavin-dependent oxidase converts CBGOA to CBCOA at about pH 5 to about pH 8. In some embodiments, the non-natural flavin-dependent oxidase converts CBGOA to CBCOA at about pH 5.5 to about pH 7.5. In some embodiments, the non-natural flavin-dependent oxidase converts CBGOA to CBCOA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGOA to CBCOA at about pH 5. In some embodiments, the non-natural flavin-dependent oxidase converts CBGOA to CBCOA at about pH 7.4 or about pH 7.5.

In some embodiments, the non-natural flavin-dependent oxidase having at least 70%, at least 80%, at least 85%, at least 90% or at least 95% sequence identity to SEQ ID NO:1 is further capable of converting CBGVA to cannabichromevarinic acid (CBCVA), cannabidivarinic acid (CBDVA), tetrahydrocannabivarin acid (THCVA), or a combination thereof. In some embodiments, the non-natural flavin-dependent oxidase converts CBGVA to CBDVA. In some embodiments, the non-natural flavin-dependent oxidase further converts CBGVA to THCVA. In some embodiments, the non-natural flavin-dependent oxidase further converts CBGVA to CBCVA. In some embodiments, the non-natural flavin-dependent oxidase converts CBGVA to CBCVA at about pH 4 to about pH 9. In some embodiments, the non-natural flavin-dependent oxidase converts CBGVA to CBCVA at about pH 4.5 to about pH 8.5. In some embodiments, the non-natural flavin-dependent oxidase converts CBGVA to CBCVA at about pH 5 to about pH 8. In some embodiments, the non-natural flavin-dependent oxidase converts CBGVA to CBCVA at about pH 5.5 to about pH 7.5. In some embodiments, the non-natural flavin-dependent oxidase converts CBGVA to CBCVA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGVA to CBCVA at about pH 5. In some embodiments, the non-natural flavin-dependent oxidase converts CBGVA to CBCVA at about pH 7.4 or about pH 7.5.

In some embodiments, the non-natural flavin-dependent oxidase having at least 70%, at least 80%, at least 85%, at least 90% or at least 95% sequence identity to SEQ ID NO:1 is further capable of converting CBG to cannabichromene (CBC), cannabidiol (CBD), tetrahydrocannabinol (THC), or a combination thereof. In some embodiments, the non-natural flavin-dependent oxidase converts CBG to CBD. In some embodiments, the non-natural flavin-dependent oxidase converts CBG to THC. In some embodiments, the non-natural flavin-dependent oxidase converts CBG to CBC. In some embodiments, the non-natural flavin-dependent oxidase converts CBG to CBC at about pH 4 to about pH 9. In some embodiments, the non-natural flavin-dependent oxidase converts CBG to CBC at about pH 4.5 to about pH 8.5. In some embodiments, the non-natural flavin-dependent oxidase converts CBG to CBC at about pH 5 to about pH 8. In some embodiments, the non-natural flavin-dependent oxidase converts CBG to CBC at about pH 5.5 to about pH 7.5. In some embodiments, the non-natural flavin-dependent oxidase converts CBG to CBC at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBG to CBC at about pH 5. In some embodiments, the non-natural flavin-dependent oxidase converts CBG to CBC at about pH 7.4 or about pH 7.5.

### Clz9

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:3. SEQ ID NO:3 describes the amino acid sequence of the Clz9 protein from Streptomyces sp. CNH-287.

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:19. SEQ ID NO:19 describes the amino acid sequence of the Clz9 protein from Streptomyces sp. CNH-287 with a 14-amino acid truncation at the N-terminus.

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:20. SEQ ID NO:20 describes the amino acid sequence of the Clz9 protein from Streptomyces sp. CNH-287 with a 29-amino acid truncation at the N-terminus.

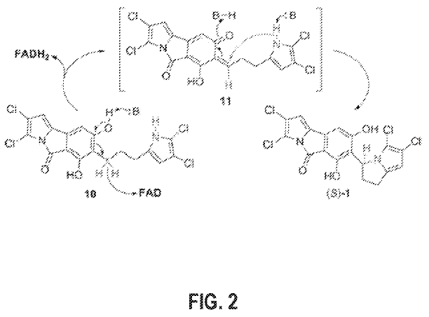

The present inventors noticed that Clz9 from Streptomyces sp. CNH-287 may have a similar catalysis mechanism as a cannabinoid synthase, e.g., THCAS, CBDAS, or CBCAS. Unless otherwise specified, “Clz9” as used herein refers to the wild-type Clz9 from Streptomyces sp. CNH-287. Clz9 is a BBE-like enzyme that catalyzes the final step of the biosynthesis of the tetrachlorinated alkaloid Chlorizidine A (see, e.g., Mantovani et al. (2013), J Am Chem Soc 135:18032-18035). The proposed reaction mechanism of Clz9 is described in FIG. 2, which shows the conversion of Compound 10 to Chlorizidine A. According to the reaction mechanism in FIG. 2, Clz9 likely deprotonates the phenolic hydroxyl of Compound 10, thereby facilitating the abstraction of the hydride by the FAD cofactor in Clz9 and generating intermediate Compound 11. Further nucleophilic attack from the pyrrole nitrogen yields the final compound, Chlorizidine A. Compound 11 contains a reactive ortho-quinone methide, which the present inventors have noticed to resemble the suggested intermediate during conversion of CBGA to cannabinoids such as THCA (see, e.g., Shoyama et al. (2012), J Mol Biol 423:96-105) and CBCA (Pollastro et al. (2018), Nat Prod Comm 13:1189-1194). The present inventors further noticed that Clz9 accepts cannabinoid precursors such as CBGA, CBGOA, CBGVA, and CBG as substrate.

In some embodiments, the non-natural flavin-dependent oxidase having at least 70%, at least 80%, at least 85%, at least 90% or at least 95% sequence identity to SEQ ID NO:3 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Clz9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Clz9. In some embodiments, the amino acid variation in the non-natural flavin-dependent oxidase is an amino acid substitution, deletion, or insertion. In some embodiments, the variation is a substitution of one or more amino acids in the wild-type Clz9 polypeptide sequence. In some embodiments, the variation is a deletion of one or more amino acids in the wild-type Clz9 polypeptide sequence. In some embodiments, the variation is an insertion of one or more amino acids in the wild-type Clz9 polypeptide sequence.

In some embodiments, the non-natural flavin-dependent oxidase having at least 70%, at least 80%, at least 85%, at least 90% or at least 95% sequence identity to SEQ ID NO:3 comprises a deletion. In some embodiments, the variation is a deletion of about 1 to 100, 1 to 90, 1 to 80, 1 to 70, 1 to 60, 1 to 50, 1 to 40, 1 to 30, 1 to 20, 1 to 10, 1 to 5, 5 to 50, 10 to 40, 10 to 38, 12 to 35, or 14 to 30 amino acids. In some embodiments, the variation is a deletion of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the variation is a deletion of an N-terminus of SEQ ID NO:3, also referred to as an “N-terminal truncation.” In some embodiments, the deletion is a deletion of about 5 to about 50 amino acid residues at the N-terminus of SEQ ID NO:3. In some embodiments, the deletion is a deletion of about 10 to about 40 amino acid residues at the N-terminus of SEQ ID NO:3. In some embodiments, the deletion is a deletion of about 12 to about 35 amino acid residues at the N-terminus of SEQ ID NO:3. In some embodiments, the deletion is a deletion of about 14 to about 30 amino acid residues at the N-terminus of SEQ ID NO:3. In some embodiments, the variation comprises a deletion of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids at the N-terminus of SEQ ID NO:3. In some embodiments, the non-natural flavin-dependent oxidase comprising a deletion at the N-terminus of SEQ ID NO:3, as described herein, has about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, or 20-fold higher rate of production of a cannabinoid, e.g., CBCA, from a prenylated aromatic compound, e.g., CBGA, as compared to a non-natural flavin-dependent oxidase of SEQ ID NO:3 that does not comprise the N-terminal deletion. In some embodiments, the non-natural flavin-dependent oxidase comprises (i) a deletion of about 5 to about 50 amino acids at the N-terminus of SEQ ID NO:3 and (ii) an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

In some embodiments, the non-natural flavin-dependent oxidase comprises an amino acid sequence having at least 70%, at least 80%, at least 85%, at least 90% or at least 95% identity to SEQ ID NO:19 or 20. In some embodiments, the non-natural flavin-dependent oxidase of SEQ ID NO:19 or 20 has about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, or 20-fold higher rate of production of a cannabinoid, e.g., CBCA, from a prenylated aromatic compound, e.g., CBGA, as compared to a non-natural flavin-dependent oxidase of SEQ ID NO:3.

In some embodiments, the non-natural flavin-dependent oxidase having at least 70%, at least 80%, at least 85%, at least 90% or at least 95% sequence identity to SEQ ID NO:19 or 20 comprises a variation at amino acid position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

It will be understood by one of ordinary skill in the art that alignment methods can be used to determine the appropriate amino acid number that corresponds to the position referenced in SEQ ID NO:3. For example, the first amino acid in SEQ ID NO:19 corresponds to the 15th amino acid of SEQ ID NO:3, and thus, position “W58” of SEQ ID NO:3 corresponds to position “W44” of SEQ ID NO:19. In another example, the first amino acid in SEQ ID NO:20 corresponds to the 30th amino acid of SEQ ID NO:3, and thus, position “W58” of SEQ ID NO:3 corresponds to position “W28” of SEQ ID NO:20.

In some embodiments, the non-natural flavin-dependent oxidase having at least 70%, at least 80%, at least 85%, at least 90% or at least 95% sequence identity to SEQ ID NO:3 comprises a variation at amino acid position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3. In some embodiments, the non-natural flavin-dependent oxidase further comprises a deletion of about 5 to about 50 amino acid residues at an N-terminus of SEQ ID NO:3. In some embodiments, the variation is in an active site of wild-type Clz9. In some embodiments, the variation is in a FAD-binding site of wild-type Clz9. In some embodiments, the variation is a substitution of the amino acid residue in wild-type Clz9 with the corresponding amino acid residue in the active site of a wild-type cannabinoid synthase from C. sativa, e.g., THCAS, CBDAS, or CBCAS as described herein.

In some embodiments, the variation at amino acid position W58 is W58Q, W58H, W58K, W58G, or W58V. In some embodiments, the variation at amino acid position M101 is M101A, M101S, M101F, or M101Y. In some embodiments, the variation at amino acid position L104 is L104M or L104H. In some embodiments, the variation at amino acid position 1160 is I160V. In some embodiments, the variation at amino acid position G161 is G161C, G161A, G161Q, or G161L. In some embodiments, the variation at amino acid position A163 is A163G. In some embodiments, the variation at amino acid position V167 is V167F. In some embodiments, the variation at amino acid position L168 is L168S or L168G. In some embodiments, the variation at amino acid position A171 is A171Y or A171F. In some embodiments, the variation at amino acid position N267 is N267V, N267M, or N267L. In some embodiments, the variation at amino acid position L269 is L269M, L269T, L269A, or L269R. In some embodiments, the variation at amino acid position I271 is I271H or I271R. In some embodiments, the variation at amino acid position Y273 is Y2731 or Y273R. In some embodiments, the variation at amino acid position Q275 is Q275K or Q275R. In some embodiments, the variation at amino acid position A281 is A281R. In some embodiments, the variation at amino acid position L283 is L283V. In some embodiments, the variation at amino acid position C285 is C285L. In some embodiments, the variation at amino acid position E287 is E287H or E287L. In some embodiments, the variation at amino acid position V323 is V323F or V323Y. In some embodiments, the variation at amino acid position V336 is V336F. In some embodiments, the variation at amino acid position A338 is A338I. In some embodiments, the variation at amino acid position G340 is G340L. In some embodiments, the variation at amino acid position L342 is L342Y. In some embodiments, the variation at amino acid position E370 is E370M or E370Q. In some embodiments, the variation at amino acid position V372 is V372A, V372E, V372I, V372L, V372T, or V372C. In some embodiments, the variation at amino acid position A398 is A398E or A398V. In some embodiments, the variation at amino acid position N400 is N400W. In some embodiments, the variation at amino acid position H402 is H402T, H402I, H402V, H402A, H402M, or H402Q. In some embodiments, the variation at amino acid position D404 is D404S, D404T, or D404A. In some embodiments, the variation at amino acid position V436 is V436L. In some embodiments, the variation at amino acid position T438 is T438A, T438Y, or T438F. Unless otherwise specified, the amino acid positions correspond to SEQ ID NO:3.

In some embodiments, the variation in the non-natural flavin-dependent oxidase having at least 70%, at least 80%, at least 85%, at least 90% or at least 95% sequence identity to SEQ ID NO:3, 19, or 20 comprises an amino acid substitution selected from W58Q, W58H, W58K, W58G, W58V, M101A, M101S, M101F, M101Y, L104M, L104H, I160V, G161C, G161A, G161Q, G161L, A163G, V167F, L168S, L168G, A171Y, A171F, N267V, N267M, N267L, L269M, L269T, L269A, L269R, I271H, I271R, Y2731, Y273R, Q275K, Q275R, A281R, L283V, C285L, E287H, E287L, V323F, V323Y, V336F, A338I, G340L, L342Y, E370M, E370Q, V372A, V372E, V372I, V372L, V372T, V372C, A398E, A398V, N400W, H402T, H402I, H402V, H402A, H402M, H402Q, D404S, D404T, D404A, V436L, T438A, T438Y, T438F, and a combination thereof, wherein the amino acid position corresponds to SEQ ID NO:3. In some embodiments, the non-natural flavin-dependent oxidase further comprises a deletion of about 5 to about 50 amino acid residues at an N-terminus of SEQ ID NO:3.

In some embodiments, the variation in the non-natural flavin-dependent oxidase having at least 70%, at least 80%, at least 85%, at least 90% or at least 95% sequence identity to SEQ ID NO:3, 19, or 20 comprises an amino acid substitution at position T438, N400, D404, or a combination thereof, wherein the position corresponds to SEQ ID NO:3. In some embodiments, the amino acid substitution comprises T438A, T438Y, N400W, D404A, or a combination thereof, wherein the amino acid position corresponds to SEQ ID NO:3. In some embodiments, the amino acid substitution comprises T438A, wherein the amino acid position corresponds to SEQ ID NO:3. In some embodiments, the amino acid substitution comprises T438Y, wherein the amino acid position corresponds to SEQ ID NO:3. In some embodiments, the amino acid substitution comprises N400W, wherein the amino acid position corresponds to SEQ ID NO:3. In some embodiments, the amino acid substitution comprises D404A, wherein the amino acid position corresponds to SEQ ID NO:3. In some embodiments, the non-natural flavin-dependent oxidase further comprises a deletion of about 5 to about 50 amino acid residues at an N-terminus of SEQ ID NO:3.

In some embodiments, the variation in the non-natural flavin-dependent oxidase having at least 70%, at least 80%, at least 85%, at least 90% or at least 95% sequence identity to SEQ ID NO:3, 19, or 20 comprises an amino acid substitution at position D404 and an amino acid substitution at position L269, Y273, Q275, L283, C285, V323, E370, V372, N400, H402, T438, or a combination thereof, wherein the amino acid position corresponds to SEQ ID NO:3. In some embodiments, the variation comprises D404A and one of: L269R, L269T, Q275R, Y273R, L283V, C285L, V323F, V323Y, E370M, E370Q, V372I, N400W, H402A, H402I, H402M, H402T, H402V, T438A, T438F, or T438Y, wherein the amino acid position corresponds to SEQ ID NO:3. In some embodiments, the variation comprises D404A and L269R, wherein the amino acid position corresponds to SEQ ID NO:3. In some embodiments, the variation comprises D404A and L269T, wherein the amino acid position corresponds to SEQ ID NO:3. In some embodiments, the variation comprises D404A and Q275R, wherein the amino acid position corresponds to SEQ ID NO:3. In some embodiments, the variation comprises D404A and Y273R, wherein the amino acid position corresponds to SEQ ID NO:3. In some embodiments, the variation comprises D404A and L283V, wherein the amino acid position corresponds to SEQ ID NO:3. In some embodiments, the variation comprises D404A and C285L, wherein the amino acid position corresponds to SEQ ID NO:3. In some embodiments, the variation comprises D404A and V323F, wherein the amino acid position corresponds to SEQ ID NO:3. In some embodiments, the variation comprises D404A and E370M, wherein the amino acid position corresponds to SEQ ID NO:3. In some embodiments, the variation comprises D404A and E370Q, wherein the amino acid position corresponds to SEQ ID NO:3. In some embodiments, the variation comprises D404A and V372I, wherein the amino acid position corresponds to SEQ ID NO:3. In some embodiments, the variation comprises D404A and N400W, wherein the amino acid position corresponds to SEQ ID NO:3. In some embodiments, the variation comprises D404A and H402A, wherein the amino acid position corresponds to SEQ ID NO:3. In some embodiments, the variation comprises D404A and H402I, wherein the amino acid position corresponds to SEQ ID NO:3. In some embodiments, the variation comprises D404A and H402M, wherein the amino acid position corresponds to SEQ ID NO:3. In some embodiments, the variation comprises D404A and H402T, wherein the amino acid position corresponds to SEQ ID NO:3. In some embodiments, the variation comprises D404A and H402V, wherein the amino acid position corresponds to SEQ ID NO:3. In some embodiments, the variation comprises D404A and T438A, wherein the amino acid position corresponds to SEQ ID NO:3. In some embodiments, the variation comprises D404A and T438F, wherein the amino acid position corresponds to SEQ ID NO:3. In some embodiments, the variation comprises D404A and T438Y, wherein the amino acid position corresponds to SEQ ID NO:3. In some embodiments, the non-natural flavin-dependent oxidase further comprises a deletion of about 5 to about 50 amino acid residues at an N-terminus of SEQ ID NO:3.

In some embodiments, the variation in the non-natural flavin-dependent oxidase having at least 70%, at least 80%, at least 85%, at least 90% or at least 95% sequence identity to SEQ ID NO:3, 19, or 20 comprises an amino acid substitution at position D404, an amino acid substitution at position T438, and an amino acid substitution at position L269, Y273, Q275, L283, C285, V323, E370, V372, N400, H402, or a combination thereof, wherein the amino acid position corresponds to SEQ ID NO:3. In some embodiments, the variation comprises D404A or D404S; T438F; and one or more of: L269R, L269T, Q275R, Y273R, L283V, C285L, V323F, V323Y, E370M, E370Q, V372I, N400W, H402A, H402I, H402M, H402T, H402V, T438A, T438F, or T438Y, wherein the amino acid position corresponds to SEQ ID NO:3. In some embodiments, the non-natural flavin-dependent oxidase further comprises a deletion of about 5 to about 50 amino acid residues at an N-terminus of SEQ ID NO:3.

In some embodiments, the variation comprises D404A, T438F, and N400W, wherein the amino acid position corresponds to SEQ ID NO:3. In some embodiments, the variation comprises D404A, T438F, and V323F, wherein the amino acid position corresponds to SEQ ID NO:3. In some embodiments, the variation comprises D404A, T438F, and V323Y, wherein the amino acid position corresponds to SEQ ID NO:3. In some embodiments, the variation comprises D404A, T438F, and E370M, wherein the amino acid position corresponds to SEQ ID NO:3. In some embodiments, the variation comprises D404A, T438F, and H402I, wherein the amino acid position corresponds to SEQ ID NO:3. In some embodiments, the variation comprises D404A, T438F, and E370Q, wherein the amino acid position corresponds to SEQ ID NO:3. In some embodiments, the variation comprises D404A, T438F, and C285L, wherein the amino acid position corresponds to SEQ ID NO:3.

In some embodiments, the variation comprises T438F, N400W, and D404S, wherein the amino acid position corresponds to SEQ ID NO:3. In some embodiments, the variation comprises T438F, V323Y, and D404S, wherein the amino acid position corresponds to SEQ ID NO:3. In some embodiments, the variation comprises T438F, H402I, and D404S, wherein the amino acid position corresponds to SEQ ID NO:3. In some embodiments, the variation comprises T438F, E370Q, and D404S, wherein the amino acid position corresponds to SEQ ID NO:3.

In some embodiments, the variation comprises D404A, T438F, V372I, and N400W, wherein the amino acid position corresponds to SEQ ID NO:3. In some embodiments, the variation comprises D404A, T438F, V323Y, and N400W, wherein the amino acid position corresponds to SEQ ID NO:3. In some embodiments, the variation comprises D404A, T438F, E370Q, and N400W, wherein the amino acid position corresponds to SEQ ID NO:3. In some embodiments, the variation comprises D404A, T438F, V323Y, and E370M, wherein the amino acid position corresponds to SEQ ID NO:3. In some embodiments, the variation comprises D404A, T438F, E370M, and N400W, wherein the amino acid position corresponds to SEQ ID NO:3.

In some embodiments, the variation comprises D404A, T438F, V323F, and H402I, wherein the amino acid position corresponds to SEQ ID NO:3. In some embodiments, the variation comprises D404A, T438F, C285L, and N400W, wherein the amino acid position corresponds to SEQ ID NO:3. In some embodiments, the variation comprises D404A, T438F, V323F, and N400W, wherein the amino acid position corresponds to SEQ ID NO:3. In some embodiments, the variation comprises D404A, T438F, E370Q, and H402T, wherein the amino acid position corresponds to SEQ ID NO:3. In some embodiments, the variation comprises D404A, T438F, N400W, and H402T, wherein the amino acid position corresponds to SEQ ID NO:3.

In some embodiments, the variation comprises D404A, T438F, V323F, and H402T, wherein the amino acid position corresponds to SEQ ID NO:3. In some embodiments, the variation comprises D404A, T438F, C285L, and V323F, wherein the amino acid position corresponds to SEQ ID NO:3. In some embodiments, the variation comprises D404A, T438F, L283V, and N400W, wherein the amino acid position corresponds to SEQ ID NO:3. In some embodiments, the variation comprises D404A, T438F, V323F, and E370M, wherein the amino acid position corresponds to SEQ ID NO:3. In some embodiments, the variation comprises D404A, T438F, Q275R, and N400W, wherein the amino acid position corresponds to SEQ ID NO:3.

In some embodiments, the variation comprises D404A, T438F, V323Y, and H402T, wherein the amino acid position corresponds to SEQ ID NO:3. In some embodiments, the variation comprises D404A, T438F, V323F, and V372I, wherein the amino acid position corresponds to SEQ ID NO:3. In some embodiments, the variation comprises D404A, T438F, C285L, and V323Y, wherein the amino acid position corresponds to SEQ ID NO:3. In some embodiments, the variation comprises D404A, T438F, E370Q, and H402I, wherein the amino acid position corresponds to SEQ ID NO:3. In some embodiments, the variation comprises D404A, T438F, V323Y, and E370Q, wherein the amino acid position corresponds to SEQ ID NO:3.

In some embodiments, the variation comprises D404A, T438F, Y273R, and V323Y, wherein the amino acid position corresponds to SEQ ID NO:3. In some embodiments, the variation comprises D404A, T438F, Y273R, and N400W, wherein the amino acid position corresponds to SEQ ID NO:3. In some embodiments, the variation comprises D404A, T438F, Y273R, and V323F, wherein the amino acid position corresponds to SEQ ID NO:3. In some embodiments, the variation comprises D404A, T438F, E370M, and H402T, wherein the amino acid position corresponds to SEQ ID NO:3. In some embodiments, the variation comprises D404A, T438F, L269T, and N400W, wherein the amino acid position corresponds to SEQ ID NO:3.

In some embodiments, the variation comprises D404A, T438F, Q275R, and V323Y, wherein the amino acid position corresponds to SEQ ID NO:3. In some embodiments, the variation comprises D404A, T438F, V323Y, and H402I, wherein the amino acid position corresponds to SEQ ID NO:3. In some embodiments, the variation comprises D404A, T438F, V323F, and E370Q, wherein the amino acid position corresponds to SEQ ID NO:3. In some embodiments, the variation comprises D404A, T438F, Y273R, and Q275R, wherein the amino acid position corresponds to SEQ ID NO:3. In some embodiments, the variation comprises D404A, T438F, C285L, and E370Q, wherein the amino acid position corresponds to SEQ ID NO:3.

In some embodiments, the variation comprises D404A, T438F, L283V, and V323Y, wherein the amino acid position corresponds to SEQ ID NO:3. In some embodiments, the variation comprises D404A, T438F, Y273R, and H402I, wherein the amino acid position corresponds to SEQ ID NO:3. In some embodiments, the variation comprises D404A, T438F, L269T, and E370M, wherein the amino acid position corresponds to SEQ ID NO:3. In some embodiments, the variation comprises D404A, T438F, C285L, and H402T, wherein the amino acid position corresponds to SEQ ID NO:3. In some embodiments, the variation comprises D404A, T438F, L269R, and N400W, wherein the amino acid position corresponds to SEQ ID NO:3.

In some embodiments, the variation comprises D404A, T438F, Y273R, and C285L, wherein the amino acid position corresponds to SEQ ID NO:3. In some embodiments, the variation comprises D404A, T438F, L283V, and H402I, wherein the amino acid position corresponds to SEQ ID NO:3. In some embodiments, the variation comprises D404A, T438F, Q275R, and E370Q, wherein the amino acid position corresponds to SEQ ID NO:3. In some embodiments, the variation comprises D404A, T438F, V372I, and H402I, wherein the amino acid position corresponds to SEQ ID NO:3. In some embodiments, the variation comprises D404A, T438F, L283V, and E370Q, wherein the amino acid position corresponds to SEQ ID NO:3. In some embodiments, the variation comprises D404A, T438F, V372I, and H402T, wherein the amino acid position corresponds to SEQ ID NO:3.

In some embodiments, variation comprises D404A, N400W, and V323Y, wherein the amino acid position corresponds to SEQ ID NO:3. In some embodiments, variation comprises D404A, T438F, N400W, and V323Y, wherein the amino acid position corresponds to SEQ ID NO:3. In some embodiments, the variation comprises an amino acid substitution at position D404, an amino acid substitution at position T438, an amino acid substitution at position N400, an amino acid substitution at position V323, and an amino acid substitution at position L269, I271, Q275, A281, L283, C285, E370, V372, H402, or a combination thereof, wherein the amino acid position corresponds to SEQ ID NO:3. In some embodiments, the variation comprises D404A, T438F, N400W, V323Y, and one or more of: L269M, I271H, Q275R, A281R, L283S, C285L, E370M, E370Q, V372I, and H402T, wherein the amino acid position corresponds to SEQ ID NO:3.

In some embodiments, the variation comprises D404A, T438F, N400W, V323Y, and E370Q, wherein the amino acid position corresponds to SEQ ID NO:3. In some embodiments, the variation comprises D404A, T438F, N400W, V323Y, and V372I, wherein the amino acid position corresponds to SEQ ID NO:3. In some embodiments, the variation comprises D404A, T438F, N400W, V323Y, and L269M, wherein the amino acid position corresponds to SEQ ID NO:3. In some embodiments, the variation comprises D404A, T438F, N400W, V323Y, and C285L, wherein the amino acid position corresponds to SEQ ID NO:3. In some embodiments, the variation comprises D404A, T438F, N400W, V323Y, and A281R, wherein the amino acid position corresponds to SEQ ID NO:3.

In some embodiments, the variation comprises D404A, T438F, N400W, V323Y, I271H, and E370Q, wherein the amino acid position corresponds to SEQ ID NO:3. In some embodiments, the variation comprises D404A, T438F, N400W, V323Y, E370Q, and V372I, wherein the amino acid position corresponds to SEQ ID NO:3. In some embodiments, the variation comprises D404A, T438F, N400W, V323Y, L269M, and E370Q, wherein the amino acid position corresponds to SEQ ID NO:3. In some embodiments, the variation comprises D404A, T438F, N400W, V323Y, C285L, and E370Q, wherein the amino acid position corresponds to SEQ ID NO:3. In some embodiments, the variation comprises D404A, T438F, N400W, V323Y, Q275R, and E370Q, wherein the amino acid position corresponds to SEQ ID NO:3.

In some embodiments, the variation comprises D404A, T438F, N400W, V323Y, L283S, and E370Q, wherein the amino acid position corresponds to SEQ ID NO:3. In some embodiments, the variation comprises D404A, T438F, N400W, V323Y, A281R, and C285L, wherein the amino acid position corresponds to SEQ ID NO:3. In some embodiments, the variation comprises D404A, T438F, N400W, V323Y, Q275R, and V372I, wherein the amino acid position corresponds to SEQ ID NO:3. In some embodiments, the variation comprises D404A, T438F, N400W, V323Y, C285L, and E370M, wherein the amino acid position corresponds to SEQ ID NO:3. In some embodiments, the variation comprises D404A, T438F, N400W, V323Y, L269M, and V372I, wherein the amino acid position corresponds to SEQ ID NO:3.

In some embodiments, the variation comprises D404A, T438F, N400W, V323Y, Q275R, and C285L, wherein the amino acid position corresponds to SEQ ID NO:3. In some embodiments, the variation comprises D404A, T438F, N400W, V323Y, I271H, and L283S, wherein the amino acid position corresponds to SEQ ID NO:3. In some embodiments, the variation comprises D404A, T438F, N400W, V323Y, Q275R, and A281R, wherein the amino acid position corresponds to SEQ ID NO:3. In some embodiments, the variation comprises D404A, T438F, N400W, V323Y, L269M, and I271H, wherein the amino acid position corresponds to SEQ ID NO:3. In some embodiments, the variation comprises D404A, T438F, N400W, V323Y, I271H, and E370M, wherein the amino acid position corresponds to SEQ ID NO:3.

In some embodiments, the variation comprises D404A, T438F, N400W, V323Y, I271H, and C285L, wherein the amino acid position corresponds to SEQ ID NO:3. In some embodiments, the variation comprises D404A, T438F, N400W, V323Y, A281R, and V372I, wherein the amino acid position corresponds to SEQ ID NO:3. In some embodiments, the variation comprises D404A, T438F, N400W, V323Y, E370M, and V372I, wherein the amino acid position corresponds to SEQ ID NO:3. In some embodiments, the variation comprises D404A, T438F, N400W, V323Y, L269M, and Q275R, wherein the amino acid position corresponds to SEQ ID NO:3. In some embodiments, the variation comprises D404A, T438F, N400W, V323Y, C285L, and V372I, wherein the amino acid position corresponds to SEQ ID NO:3.

In some embodiments, the variation comprises D404A, T438F, N400W, V323Y, V372I, and H402T, wherein the amino acid position corresponds to SEQ ID NO:3. In some embodiments, the variation comprises D404A, T438F, N400W, V323Y, L269M, and E370M, wherein the amino acid position corresponds to SEQ ID NO:3. In some embodiments, the variation comprises D404A, T438F, N400W, V323Y, Q275R, and E370M, wherein the amino acid position corresponds to SEQ ID NO:3. In some embodiments, the variation comprises D404A, T438F, N400W, V323Y, A281R, and E370Q, wherein the amino acid position corresponds to SEQ ID NO:3. In some embodiments, the variation comprises D404A, T438F, N400W, V323Y, A281R, and L283S, wherein the amino acid position corresponds to SEQ ID NO:3.

In some embodiments, the non-natural flavin-dependent oxidase having at least 70%, at least 80%, at least 85%, at least 90% or at least 95% sequence identity to SEQ ID NO:3, 19, or 20 does not comprise a variation in an active site of the flavin-dependent oxidase. As described herein, the active site of the flavin-dependent oxidase can include one or more amino acid residues involved in binding substrate (e.g., CBGA, CBGOA, CBGVA, and/or CBG), or the active site can include one or more amino acid residues involved in binding FAD cofactor, or the active site can include one or more amino acid residues involved in catalysis, e.g., conversion of CBGA into CBCA. In some embodiments, Y374, Y435, and N437 are in the active site of Clz9. In some embodiments, the non-natural flavin-dependent oxidase does not comprise a variation at any of amino acid positions Y374, Y435, and N437, wherein the amino acid position corresponds to SEQ ID NO:3. In some embodiments, the non-natural flavin-dependent oxidase does not comprise a variation at Y374. In some embodiments, the non-natural flavin-dependent oxidase does not comprise a variation at Y435. In some embodiments, the non-natural flavin-dependent oxidase does not comprise a variation at N437. In some embodiments, the non-natural flavin-dependent oxidase comprises a functionally identical or functionally similar amino acid substitution at Y374, Y435, N437, or a combination thereof. Functionally identical and functionally similar amino acid substitutions are described herein. For example, a functionally similar amino acid substitution for asparagine (N) can be glutamine (Q).

In some embodiments, the non-natural flavin-dependent oxidase having at least 70%, at least 80%, at least 85%, at least 90% or at least 95% sequence identity to SEQ ID NO:3, 19, or 20 is capable of converting CBGA to CBCA, THCA, CBDA, or a combination thereof. In some embodiments, the non-natural flavin-dependent oxidase converts CBGA to CBDA. In some embodiments, the non-natural flavin-dependent oxidase converts CBGA to THCA. In some embodiments, the non-natural flavin-dependent oxidase converts CBGA to CBCA. In some embodiments, the non-natural flavin-dependent oxidase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least or about 99%, or at least about 100% of the catalytic activity of a wild-type CBCAS. Comparison of catalytic activity is described in embodiments herein.

In some embodiments, the non-natural flavin-dependent oxidase having at least 70%, at least 80%, at least 85%, at least 90% or at least 95% sequence identity to SEQ ID NO:3, 19, or 20 converts CBGA to CBCA at about pH 4 to about pH 9. In some embodiments, the non-natural flavin-dependent oxidase converts CBGA to CBCA at about pH 4.5 to about pH 8.5. In some embodiments, the non-natural flavin-dependent oxidase converts CBGA to CBCA at about pH 5 to about pH 8. In some embodiments, the non-natural flavin-dependent oxidase converts CBGA to CBCA at about pH 5.5 to about pH 7.5. In some embodiments, the non-natural flavin-dependent oxidase converts CBGA to CBCA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGA to CBCA at about pH 5. In some embodiments, the non-natural flavin-dependent oxidase converts CBGA to CBCA at about pH 7.4 or about pH 7.5.

In some embodiments, the non-natural flavin-dependent oxidase having at least 70%, at least 80%, at least 85%, at least 90% or at least 95% sequence identity to SEQ ID NO:3, 19, or 20 is further capable of converting CBGOA to CBCOA, CBDOA, THCOA, or a combination thereof. In some embodiments, the non-natural flavin-dependent oxidase converts CBGOA to CBDOA. In some embodiments, the non-natural flavin-dependent oxidase converts CBGOA to THCOA. In some embodiments, the non-natural flavin-dependent oxidase converts CBGOA to CBCOA. In some embodiments, the non-natural flavin-dependent oxidase converts CBGOA to CBCOA at about pH 4 to about pH 9. In some embodiments, the non-natural flavin-dependent oxidase converts CBGOA to CBCOA at about pH 4.5 to about pH 8.5. In some embodiments, the non-natural flavin-dependent oxidase converts CBGOA to CBCOA at about pH 5 to about pH 8. In some embodiments, the non-natural flavin-dependent oxidase converts CBGOA to CBCOA at about pH 5.5 to about pH 7.5. In some embodiments, the non-natural flavin-dependent oxidase converts CBGOA to CBCOA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGOA to CBCOA at about pH 5. In some embodiments, the non-natural flavin-dependent oxidase converts CBGOA to CBCOA at about pH 7.4 or about pH 7.5.

In some embodiments, the non-natural flavin-dependent oxidase having at least 70%, at least 80%, at least 85%, at least 90% or at least 95% sequence identity to SEQ ID NO:3, 19, or 20 is further capable of converting CBGVA to CBCVA, CBDVA, THCVA, or a combination thereof. In some embodiments, the non-natural flavin-dependent oxidase converts CBGVA to CBDVA. In some embodiments, the non-natural flavin-dependent oxidase further converts CBGVA to THCVA. In some embodiments, the non-natural flavin-dependent oxidase further converts CBGVA to CBCVA. In some embodiments, the non-natural flavin-dependent oxidase converts CBGVA to CBCVA at about pH 4 to about pH 9. In some embodiments, the non-natural flavin-dependent oxidase converts CBGVA to CBCVA at about pH 4.5 to about pH 8.5. In some embodiments, the non-natural flavin-dependent oxidase converts CBGVA to CBCVA at about pH 5 to about pH 8. In some embodiments, the non-natural flavin-dependent oxidase converts CBGVA to CBCVA at about pH 5.5 to about pH 7.5. In some embodiments, the non-natural flavin-dependent oxidase converts CBGVA to CBCVA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGVA to CBCVA at about pH 5. In some embodiments, the non-natural flavin-dependent oxidase converts CBGVA to CBCVA at about pH 7.4 or about pH 7.5.

In some embodiments, the non-natural flavin-dependent oxidase having at least 70%, at least 80%, at least 85%, at least 90% or at least 95% sequence identity to SEQ ID NO:3, 19, or 20 is further capable of converting CBG to CBC, CBD, THC, or a combination thereof. In some embodiments, the non-natural flavin-dependent oxidase converts CBG to CBD. In some embodiments, the non-natural flavin-dependent oxidase converts CBG to THC. In some embodiments, the non-natural flavin-dependent oxidase converts CBG to CBC. In some embodiments, the non-natural flavin-dependent oxidase converts CBG to CBC at about pH 4 to about pH 9. In some embodiments, the non-natural flavin-dependent oxidase converts CBG to CBC at about pH 4.5 to about pH 8.5. In some embodiments, the non-natural flavin-dependent oxidase converts CBG to CBC at about pH 5 to about pH 8. In some embodiments, the non-natural flavin-dependent oxidase converts CBG to CBC at about pH 5.5 to about pH 7.5. In some embodiments, the non-natural flavin-dependent oxidase converts CBG to CBC at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBG to CBC at about pH 5. In some embodiments, the non-natural flavin-dependent oxidase converts CBG to CBC at about pH 7.4 or about pH 7.5.

In some embodiments, the non-natural flavin-dependent oxidase provided herein further comprises an affinity tag, a purification tag, a solubility tag, or a combination thereof. As used in the context of proteins and polypeptides, a “tag” can refer to a short polypeptide sequence, typically about 5 to about 50 amino acids in length, that is covalently attached to the protein of interest, e.g., the non-natural flavin-dependent oxidase. Additionally or alternatively, a tag can also comprise a polypeptide that is greater than 50 amino acids in length and that provides a desired property, e.g., increases solubility, to the tagged protein of interest. In some embodiments, the tag is attached to the protein such that it in the same reading frame as the protein, i.e., “in-frame.” In general, the tag allows a specific chemical or enzymatic modification to the protein of interest. Solubility tags increases the solubility of the tagged protein and include, e.g., thioredoxin (TRX), poly(NANP), maltose-binding protein (MBP), and glutathione S-transferase (GST). Affinity tags allow the protein to bind to a specific molecule. Examples of affinity tags include chitin binding protein (CBP), Strep-tag, poly(His) tag, and the like; in addition, certain solubility tags such as MBP and GST can also serve as an affinity tag. Purification tags, also termed chromatography tags, allow the protein to be separated from other components in a particular purification or separation technique and are typically comprise polyanionic amino acids, such as the FLAG-tag. Further examples of tags that can be included on the non-natural flavin-dependent oxidases provided herein include, without limitation, epitope tags such as ALFA-tag, V5-tag, Myc-tag, HA-tag, Spot-tag, T7-tag, and NE-tag, which can be useful in western blotting or immunoprecipitation; and fluorescence tags such as GFP and its variants for visualization of the tagged protein. One of ordinary skill in the art would understand that the non-natural flavin-dependent oxidase provided herein can comprise a single tag, or a combination of tags including multiple functions. Methods of producing tagged proteins, e.g., a tagged non-natural flavin-dependent oxidase, are known in the field. See, e.g., Kimple et al. (2013), Curr Protoc Protein Sci 73: Unit-9.9.

In some embodiments, the disclosure further provides a polynucleotide comprising a nucleic acid sequence encoding the non-natural flavin-dependent oxidase described herein. In some embodiments, the disclosure further provides a polynucleotide comprising: (a) a nucleic acid sequence encoding a polypeptide having at least 80% sequence identity to SEQ ID NO:1, 3, 19, or 20; and (b) a heterologous regulatory element operably linked to the nucleic acid sequence. In some embodiments, the nucleic acid sequence encodes a polypeptide having at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:1. In some embodiments, the nucleic acid sequence encodes a polypeptide having at least 85%, at least 90%, at least 91%, at least 92%, at least 93%, at least 94%, at least 95%, at least 96%, at least 97%, at least 98%, at least 99%, or 100% sequence identity to SEQ ID NO:3. In some embodiments, the polynucleotide encodes a polypeptide having at least 70%, at least 80%, at least 85%, at least 90% or at least 95% identity to SEQ ID NO:1 and comprising an amino acid substitution at position V136, S137, T139, L144, Y249, F313, Q353, or a combination thereof. In some embodiments, the polynucleotide encodes a polypeptide having at least 70%, at least 80%, at least 85%, at least 90% or at least 95% identity to SEQ ID NO:3 and comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof. In some embodiments, the polynucleotide encodes a polypeptide having at least 70%, at least 80%, at least 85%, at least 90% or at least 95% identity to SEQ ID NO:3 and comprising a deletion of about 5 to about 50, about 10 to about 40, about 12 to about 35, or about 14 to about 30 amino acid residues at an N-terminus of SEQ ID NO:3, and optionally further comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3. In some embodiments, the polynucleotide encodes a polypeptide having at least 70%, at least 80%, at least 85%, at least 90% or at least 95% identity to SEQ ID NO:19 or 20, and optionally comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

In some embodiments, the disclosure further provides a polynucleotide comprising any one of SEQ ID NOs:12-15. In some embodiments, the polynucleotide comprises a nucleic acid sequence having at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:12-15.

In some embodiments, the nucleic acid sequence encoding the non-natural flavin-dependent oxidase is codon optimized. An example of a codon optimized sequence is, in one instance, a sequence optimized for expression in a bacterial host cell, e.g., E. coli. In some embodiments, one or more codons in a nucleic acid sequence encoding the non-natural flavin-dependent oxidase described herein corresponds to the most frequently used codon for a particular amino acid in the bacterial host cell.

In some embodiments, the heterologous regulatory element of the polynucleotide comprises a promoter, an enhancer, a silencer, a response element, or a combination thereof. In some embodiments, the heterologous regulatory element of (b) is a bacterial regulatory element. Non-limiting examples of bacterial regulatory elements include the T7 promoter, Sp6 promoter, lac promoter, araBad promoter, trp promoter, and Ptac promoter. Further examples of regulatory elements can be found, e.g., using the PRODORIC2 database (Eckweiler et al. (2018), Nucleic Acids Res 46(D1):D320-D326).

In some embodiments, the disclosure provides an expression construct comprising the polynucleotide provided herein. Expression constructs are described herein and include, e.g., pQE vectors (Qiagen), pBluescript plasmids, pNH vectors, lambda-ZAP vectors (Stratagene); pTrc99a, pKK223-3, pDR540, and pRIT2T (Pharmacia). In some embodiments, the expression construct comprises a regulatory element. Regulatory elements are provided herein.

In some embodiments, the disclosure provides an engineered cell comprising the non-natural flavin-dependent oxidase described herein, the polynucleotide described herein, the expression construct comprising the polynucleotide described herein, or a combination thereof. In some embodiments, the engineered cell comprises the non-natural flavin-dependent oxidase. In some embodiments, the engineered cell comprises the polynucleotide comprising a nucleic acid sequence encoding the non-natural flavin-dependent oxidase. In some embodiments, the engineered cell comprises the polynucleotide comprising (a) a nucleic acid sequence encoding a polypeptide having at least 80% sequence identity to SEQ ID NO:1, 3, 19, or 20; and (b) a heterologous regulatory element operably linked to the nucleic acid sequence. In some embodiments, the engineered cell comprises the expression construct comprising the polynucleotide provided herein. In some embodiments, the polynucleotide encodes a polypeptide having at least 70%, at least 80%, at least 85%, at least 90% or at least 95% identity to SEQ ID NO:1 and comprising an amino acid substitution at position V136, S137, T139, L144, Y249, F313, Q353, or a combination thereof. In some embodiments, the polynucleotide encodes a polypeptide having at least 70%, at least 80%, at least 85%, at least 90% or at least 95% identity to SEQ ID NO:3 and comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof. In some embodiments, the polynucleotide encodes a polypeptide having at least 70%, at least 80%, at least 85%, at least 90% or at least 95% identity to SEQ ID NO:3 and comprising a deletion of about 5 to about 50, about 10 to about 40, about 12 to about 35, or about 14 to about 30 amino acid residues at an N-terminus of SEQ ID NO:3, and optionally further comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof. In some embodiments, the polynucleotide encodes a polypeptide having at least 70%, at least 80%, at least 85%, at least 90% or at least 95% identity to SEQ ID NO:19 or 20, and optionally further comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof. In some embodiments, the disclosure provides a method of making an isolated non-natural flavin-dependent oxidase, comprising isolating the non-natural flavin-dependent oxidase expressed in the engineered cell provided herein. In some embodiments, the disclosure provides an isolated non-natural flavin-dependent oxidase, wherein the isolated non-natural flavin-dependent oxidase is expressed and isolated from the engineered cell.

In some embodiments, the engineered cell further comprises a cannabinoid biosynthesis pathway enzyme. An exemplary cannabinoid biosynthesis pathway starts from the conversion of hexanoate to hexanoyl-CoA (Hex-CoA) via hexanoyl-CoA synthetase. Hex-CoA is then converted to 3-oxooctanoyl-CoA, then 3,5-dioxodecanoyl-CoA, then 3,5,7-trioxododecanoyl-CoA by olivetol synthase (OLS; also known as tetraketide synthase or TKS), which is subsequently converted to olivetolic acid by olivetolic acid cyclase (OAC). A prenyltransferase then catalyzes the reaction between olivetolic acid and geranyldiphosphate (GPP) to produce CBGA, which can be converted to CBG via non-enzymatic decarboxylation. In an analogous manner, CBGOA is produced from the prenyltransferase-catalyzed reaction between orsellinic acid and GPP; CBGVA is produced from the prenyltransferase-catalyzed reaction between divarinic acid and GPP. In some embodiments, the CBGA, CBG, CBGOA, and/or CBGVA produced from the cannabinoid biosynthesis pathways are further converted into a cannabinoid by the non-natural flavin-dependent oxidases provided herein. Cannabinoid biosynthesis pathways are further described, e.g., in Degenhardt et al., Chapter 2—The Biosynthesis of Cannabinoids. Handbook of Cannabis and Related Pathologies, pp. 13-23; Elsevier Academic Press, 2017. In some embodiments, the cannabinoid biosynthesis pathway enzyme comprises an enzyme from Cannabis sativa, e.g., olivetol synthase (OLS), olivetolic acid cyclase (OAC), a geranyl pyrophosphate (GPP) pathway enzyme, and/or prenyltransferase. In some embodiments, the cannabinoid biosynthesis pathway enzyme comprises a homolog of a C. sativa enzyme, e.g., a homolog of OLS, OAC, GPP pathway enzyme, and/or prenyltransferase. It will be understood by one of ordinary skill in the art that a homolog of a cannabinoid biosynthesis pathway enzyme can be a sequence homolog, a structural homolog, and/or an enzyme activity homolog.

In some embodiments, the engineered cell further comprises an enzyme in the CBGA biosynthesis pathway. In some embodiments, the engineered cell further comprises an enzyme in the CBG biosynthesis pathway. In some embodiments, the engineered cell comprises an enzyme in the CBGOA biosynthesis pathway. In some embodiments, the engineered cell comprises an enzyme in the CBGVA biosynthesis pathway. In some embodiments, the cannabinoid biosynthesis pathway enzyme of the engineered cell comprises OLS, OAC, prenyltransferase, or a combination thereof.

In some embodiments, CBGA is produced from olivetolic acid (OA) and geranyldiphosphate (GPP). In some embodiments, CBG is produced from CBGA. In some embodiments, CBGOA is produced from orsellinic acid (OSA) and GPP. In some embodiments, CBGVA is produced from divarinic acid (DA) and GPP. In some embodiments, the engineered cells of the disclosure have higher levels of available GPP, OA, OSA, DA, CBGA, CBG, CBGOA, and/or CBGVA (and derivatives or analogs thereof) as compared to a naturally-occurring, non-engineered cell.

### OLS

In some embodiments, the engineered cell of the present disclosure further comprises an enzyme in the olivetolic acid pathway. In some embodiments, the enzyme in the olivetolic acid pathway is olivetol synthase (OLS). OLS catalyzes the addition of two malonyl-CoA (Mal-CoA) and hexanoyl-CoA (Hex-CoA) to form 3,5-dioxodecanoyl-CoA, which can be further converted by OLS to 3,5,7-trioxododecanoyl-CoA with the addition of a third Mal-CoA. 3,5,7-trioxododecanoyl-CoA can subsequently be converted to OA by OAC.

Although the metabolic pathway is discussed herein with reference to certain precursors and intermediates, it is understood that analogs may be substituted in essentially the same reactions. For example, it is understood that Hex-CoA analogs, including other acyl-CoA, can be used in place of Hex-CoA. Exemplary analogs include, but are not limited to any C₂-C₂₀ acyl-CoA such as acetyl-CoA, propionyl-CoA, butyryl-CoA, pentanoyl-CoA, heptanoyl-CoA, octanoyl-CoA, nonanoyl-CoA, decanoyl-CoA, and aromatic acid CoA such as benzoic, chorismic, phenylacetic, and phenoxyacetic acid-CoA.

In some embodiments, the engineered cells of the disclosure have increased production of one or more precursors (e.g., Mal-CoA, Hex-CoA or other acyl-CoA, OA, OSA, DA, CBGA, CBGOA, and/or CBGVA) of the cannabinoids provided herein, e.g., CBCA, CBDA, THCA, CBCOA, CBDOA, THCOA, CBCVA, CBCOA, THCOA, CBC, CBD, and/or THC. In some embodiments, the engineered cells of the disclosure have increased production of one or more precursors (e.g., Mal-CoA, Hex-CoA or other acyl-CoA, OA, OSA, DA, CBGA, CBGOA, and/or CBGVA) of CBCA, THCA, CBCOA, CBCVA, and/or CBC.

In some embodiments, the engineered cells of the disclosure have increased production of OA precursors, e.g., Mal-CoA and/or acyl-CoA (such as, e.g., Hex-CoA or any other acyl-CoA described herein). In some embodiments, the non-natural OLS preferentially catalyzes the condensation of Mal-CoA and acyl-CoA (such as, e.g., Hex-CoA or any other acyl-CoA described herein) to form a polyketide (such as, e.g., 3,5,7-trioxododecanoyl-CoA and 3,5,7-trioxododecanoate and their analogs) over the reaction side products, e.g., pentyl diacetic acid lactone (PDAL), hexanoyl triacetic acid lactone (HTAL), or other lactone analogs compared with a wild-type OLS.

In some embodiments, the engineered cell expresses an exogenous or overexpresses an exogenous or endogenous OLS. In some embodiments, the OLS is a natural OLS, e.g., a wild-type OLS. In some embodiments, the OLS is a non-natural OLS. In some embodiments, the OLS comprises one or more amino acid substitutions relative to a wild-type OLS. In some embodiments, the one or more amino acid substitutions in the non-natural OLS increases the activity of the OLS as compared to a wild-type OLS.

In some embodiments, the OLS has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:7.

In some embodiments, the OLS comprises a variation at amino acid position A125, S126, D185, M187, L190, G204, G209, D210, G211, G249, G250, L257, F259, M331, S332, or a combination thereof, wherein the position corresponds to SEQ ID NO:7. In some embodiments, the variation is an amino acid substitution. OLS and non-natural variants thereof are further discussed in, e.g., WO2020/214951.

In some embodiments, the non-natural OLS comprises an amino acid substitution selected from A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M1871, M187K, M187R, L190G, L190A, L190S, L190P, L190C, L190T, L190D, L190N, L190E, L190Q, L190H, L190V, L190M, L190I, L190K, L190R, G204A, G204C, G204P, G204V, G204L, G2041, G204M, G204F, G204W, G204S, G204T, G204Y, G204H, G204N, G204Q, G204D, G204E, G204K, G204R, G209A, G209C, G209P, G209V, G209L, G2091, G209M, G209F, G209W, G209S, G209T, G209Y, G209H, G209N, G209Q, G209D, G209E, G209K, G209R, D210A, D210C, D210P, D210V, D210L, D2101, D210M, D210F, D210W, D210S, D210T, D210Y, D210H, D210N, D210Q, D210E, D210K, D210R, G211A, G211C, G211P, G211V, G211L, G211I, G211M, G211F, G211W, G211S, G211T, G211Y, G211H, G211N, G211Q, G211D, G211E, G211K, G211R, G249A, G249C, G249P, G249V, G249L, G2491, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M331I, M331K, M331R, S332G, S332A, or a combination thereof, wherein the position corresponds to SEQ ID NO:7.

In some embodiments, the disclosure provides a composition comprising the non-natural flavin-dependent oxidase described herein and the OLS described herein. In some embodiments, the disclosure provides an engineered cell comprising the non-natural flavin-dependent oxidase described herein and the OLS described herein. In some embodiments, the disclosure provides one or more polynucleotides comprising one or more nucleic acid sequences encoding the non-natural flavin-dependent oxidase described herein and the OLS described herein. In some embodiments, the OLS is a non-natural OLS. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDOA, CBC, CBD, and/or THC. In some embodiments, the engineered cell produces CBCA, THCA, CBCOA, CBCVA, and/or CBC.

### OAC

In some embodiments, the engineered cell of the present disclosure further comprises an enzyme in the olivetolic acid pathway. In some embodiments, the enzyme in the olivetolic acid pathway is olivetolic acid cyclase (OAC). As discussed herein, OAC catalyzes the conversion of 3,5,7-trioxododecanoyl-CoA to OA.

In some embodiments, the engineered cell expresses an exogenous or overexpresses an exogenous or endogenous OAC. In some embodiments, the OAC is a natural OAC, e.g., a wild-type OAC. In some embodiments, the OAC is a non-natural OAC. In some embodiments, the OAC comprises one or more amino acid substitutions relative to a wild-type OAC. In some embodiments, the one or more amino acid substitutions in the non-natural OAC increases the activity of the OAC as compared to a wild-type OAC. OAC and non-natural variants thereof are further discussed in, e.g., WO2020/247741.

In some embodiments, the OAC has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:8.

In some embodiments, the OAC comprises a variation at amino acid position L9, F23, V59, V61, V66, E67, 169, Q70, I73, I74, V79, G80, F81, G82, D83, R86, W89, L92, or 194, V46, T47, Q48, K49, N50, K51, V46, T47, Q48, K49, N50, K51, or a combination thereof, wherein the position corresponds to SEQ ID NO:8. In some embodiments, the variation is an amino acid substitution. In some embodiments, the variation is in a first peptide (e.g., a first monomer) of an OAC dimer. In some embodiments, the variation is in a second peptide (e.g., a second monomer) of an OAC dimer.

In some embodiments, the OAC forms a dimer, wherein a first peptide of the dimer (e.g., a first monomer) of the dimer comprises a variation at amino acid position H5, I7, L9, F23, F24, Y27, V59, V61, V66, E67, 169, Q70, I73, I74, V79, G80, F81, G82, D83, R86, W89, L92, I94, D96, V46, T47, Q48, K49, N50, K51, or combination thereof, and wherein a second peptide (e.g., a second monomer) of the dimer comprises a variation at amino acid position V46, T47, Q48, K49, N50, K51, or combination thereof, wherein the position corresponds to SEQ ID NO:8. In some embodiments, the OAC forms a dimer, wherein a first peptide of the dimer comprises a variation at amino acid position L9, F23, V59, V61, V66, E67, 169, Q70, I73, I74, V79, G80, F81, G82, D83, R86, W89, L92, I94, V46, T47, Q48, K49, N50, K51, or combination thereof, and a second peptide of the dimer comprises a variation at amino acid position V46, T47, Q48, K49, N50, K51, or combination thereof, wherein the position corresponds to SEQ ID NO:8.

In some embodiments, the OAC comprises an amino acid substitution selected from H5X¹, wherein X¹ is G, A, C, P, V, L, I, M, F, Y, W, Q, E, K, R, S, T, Y, N, Q, D, E, K, or R; I7X², wherein X² is G, A, C, P, V, L, M, F, Y, W, K, R, S, T, H, N, Q, D, or E; L9X³, wherein X³ is G, A, C, P, V, I, M, F, Y, W, K, R, S, T, Y, H, N, Q, D, E, K, or R; F23X⁴, wherein X⁴ is G, A, C, P, V, L, I, M, Y, W, S, T, H, N, Q, D, E, K, or R; F24X⁵, wherein X⁵ is G, A, C, P, V, I, M, Y, S, T, H, N, Q, D, E, K, R, or W; Y27X⁶, wherein X⁶ is G, A, C, P, V, L, I, M, F, W, S, T, H, N, Q, D, E, K, or R; V59X⁷, wherein X⁷ is G, A, C, P, L, I, M, F, Y, W, H, Q, E, K, or R; V61X⁸, wherein X⁸ is G, A, C, P, L, I, M, F, Y, W, H, Q, E, K, R, S, T, N, or D; V66X⁹, wherein X⁹ is G, A, C, P, L, I, M, F, Y, or W; E67X¹⁰, wherein X¹⁰ is G, A, C, P, V, L, I, M, F, Y, or W; 169X¹¹, wherein X¹¹ is G, A, C, P, V, L, M, F, Y, or W; Q70X¹², wherein X¹² is S, T, H, N, D, E, R, K, or Y; I73X¹³, wherein X¹³ is G, A, C, P, V, L, M, F, Y, or W; I74X¹⁴, wherein X¹⁴ is G, A, C, P, V, L, M, F, Y, or W; V79X¹⁵, wherein X¹⁵ is G, A, C, P, L, I, M, F, Y, or W; G80X¹⁶, wherein X¹⁶ is A, C, P, V, L, I, M, F, Y, W, S, T, H, N, Q, D, E, K, or R; F81X¹⁷, wherein X¹⁷ is G, A, C, P, V, L, I, M, Y, W, S, T, H, N, Q, D, E, R, or K; G82X¹⁸, wherein X¹⁸ is A, C, P, V, L, I, M, F, Y, W, S, T, H, N, Q, E, K, or R; D83X¹⁹, wherein X¹⁹ is S, T, H, Q, N, E, R, K, or Y; R86X²⁰, wherein X²⁰ is S, T, H, Q, N, D, E, K, or Y; W89X²¹, wherein X²¹ is G, A, C, P, V, L, I, M, F, Y, W, S, T, H, N, Q, D, E, K, or R; L92X²², wherein X²² is G, A, C, P, V, I, M, F, Y, or W; I94X²³, wherein X²³ is G, A, C, P, V, L, M, F, Y, W, K, R, S, T, Y, H, N, Q, D, or E; D96X²⁴, wherein X²⁴ is S, T, H, Q, N, E, R, K, or Y; V46X²⁵, wherein X²⁵ is G, A, C, P, L, I, M, F, Y, or W; T47X²⁶, wherein X²⁶ is S, H, Q, N, D, E, R, K, or Y; Q48X²⁷, wherein X²⁷ is S, T, H, N, D, E, R, K, or Y; K49X²⁸, wherein X²⁸ is S, T, H, Q, N, D, E, R, or Y; N50X²⁹, wherein X²⁹ is G, A, C, P, V, L, I, M, F, Y, or W; K51X³⁰, wherein X³⁰ is S, T, H, Q, N, D, E, R, or Y; V46*X³¹, wherein X³¹ is G, A, C, P, L, I, M, F, Y, or W; T47*X³², wherein X³² is S, H, Q, N, D, E, R, K, or Y; Q48*X³³, wherein X³³ is S, T, H, N, D, E, R, K, or Y; K49*X³⁴, wherein X³⁴ is S, T, H, Q, N, D, E, R, or Y; N50*X³⁵, wherein X³⁵ is G, A, C, P, V, L, I, M, F, Y, or W; K51*X³⁶, wherein X³⁶ is S, T, H, Q, N, D, E, R, or Y; and a combination thereof; wherein the amino acid position corresponds to SEQ ID NO:8, and wherein the “*” following the amino acid position indicates amino acid residues from a second peptide of a OAC dimer (e.g., monomer B) and corresponding to SEQ ID NO:8.

In some embodiments, the OAC comprises more than one amino acid variations. In some embodiments, the OAC is not a single substitution at position K4A, H5A, H5L, H5Q, H5S, H5N, HSD, I7L, I7F, L9A, L9W, K12A, F23A, F231, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, wherein the amino acid position corresponds to SEQ ID NO:8.

In some embodiments, the OAC described herein is capable of producing olivetolic acid at a faster rate compared with a wild-type OAC. In some embodiments, the OAC has increased affinity for a polyketide (e.g., 3,5,7-trioxododecanoyl-CoA or an analog thereof, as produced by an OLS described herein) compared with a wild-type OAC. In some embodiments, the rate of formation of olivetolic acid from 3,5,7-trioxododecanoyl-CoA or analog thereof by the OAC described herein is about 1.2 times to about 300 times, about 1.5 times to about 200 times, or about 2 times to about 30 times as compared to a wild-type OAC. The rate of formation of olivetolic acid from 3,5,7-trioxododecanoyl-CoA or an analog thereof can be determined in an in vitro enzymatic reaction using a purified OAC. Methods of determining enzyme kinetics and product formation rate are known in the field.

In some embodiments, the OLS described herein is enzymatically capable of at least about 1.1, 1.2, 1.5, 2, 3, 4, 5, 6, 7, 8, 9, 10, 12, 15, 20, or greater rate of formation of OA and/or olivetol from Mal-CoA and Hex-CoA in the presence of an excess of the OAC described herein, as compared to a wild type OLS.

In some embodiments, the OAC is present in molar excess of the OLS in the engineered cell. In some embodiments, the molar ratio of the OLS to the OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or 1 to more than 10,000. In some embodiments, the molar ratio of the OLS to the OAC is about 1000:1, 500:1, 100:1, 10:1, 5:1, 2.5:1. 1.5:1, 1.2:1. 1.1:1, 1:1, or less than 1 to 1. In some embodiments, the enzyme turnover rate of the OAC is greater than OLS. As used herein, “turnover rate” refers to the rate at which an enzyme can catalyze a reaction (e.g., turn substrate into product). In some embodiments, the higher turnover rate of OAC compared to OLS provides a greater rate of formation of OA than olivetol.

In some embodiments, the total byproducts (e.g., olivetol and analogs thereof, PDAL, HTAL, and other lactone analogs) of the OLS reaction products in the presence of molar excess of OAC, are in an amount (w/w) of less than about 50%, 45%, 40%, 35%, 30%, 25%, 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by the combination of individual OLS and OAC enzyme reactions.

In some embodiments, the disclosure provides a composition comprising the non-natural flavin-dependent oxidase described herein and one or both of the OLS described herein and the OAC described herein. In some embodiments, the disclosure provides an engineered cell comprising the non-natural flavin-dependent oxidase described herein and one or both of the OLS described herein and the OAC described herein. In some embodiments, the disclosure provides one or more polynucleotides comprising one or more nucleic acid sequences encoding the non-natural flavin-dependent oxidase described herein and one or both of the OLS described herein and the OAC described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDOA, CBC, CBD, and/or THC. In some embodiments, the engineered cell produces CBCA, THCA, CBCOA, CBCVA, and/or CBC.

### GPP

In some embodiments, the engineered cell of the present disclosure further comprises an enzyme in the geranyl pyrophosphate (GPP) pathway. GPP pathways are further provided, e.g., in WO 2017/161041. In some embodiments, the GPP pathway comprises a mevalonate (MVA) pathway, a non-mevalonate methylerythritol-4-phosphate (MEP) pathway, an alternative non-MEP, non-MVA geranyl pyrophosphate pathway, or a combination thereof. In some embodiments, the GPP pathway comprises an enzyme selected from geranyl pyrophosphate (GPP) synthase, farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, or a combination thereof. In some embodiments, the alternative non-MEP, non-MVA geranyl pyrophosphate pathway comprises alcohol kinase, alcohol diphosphokinase, phosphate kinase, isopentenyl disphosphate isomerase, geranyl pyrophosphate synthase, or a combination thereof.

GPP and its precursors may be produced from several pathways within a host cell, including the mevalonate pathway (MVA) or a non-mevalonate, methylerythritol-4-phosphate (MEP) pathway (also known as the deoxyxylulose-5-phosphate pathway), which produce isopentenyl pyrophosphate (IPP) and dimethylallyl pyrophosphate (DMAPP), which are isomerized by isopentenyl-diphosphate delta-isomerase (IDI) and converted to GPP using geranyl pyrophosphate synthase (GPPS). As described herein, prenyltransferase can convert GPP and OA into CBGA, which can then be converted into CBCA and/or THCA by the non-natural flavin-dependent oxidase described herein. Prenyltransferase can also convert GPP and OSA into CBGOA, which can then be converted in CBCOA by the non-natural flavin-dependent oxidase described herein. Prenyltransferase can further convert GPP and DA into CBGVA, which can then be converted into CBCVA by the non-natural flavin-dependent oxidase described herein.

In some embodiments, the engineered cell produces GPP from a MVA pathway. In some embodiments, the engineered cell produces GPP from a MEP pathway. In some embodiments, the engineered cell expresses an exogenous or overexpresses an exogenous or endogenous gene that encodes any one of the enzymes in the MVA pathway or the MEP pathway, thereby increasing the production of GPP. In some embodiments, the MVA pathway enzyme is acetoacetyl-CoA thiolase (AACT); HMG-CoA synthase (HMGS); HMG-CoA reductase (HMGR); mevalonate-3-kinase (MVK); phosphomevalonate kinase (PMK); mevalonate-5-pyrophosphate decarboxylase (MVD); isopentenyl pyrophosphate isomerase (IDI), or geranyl pyrophosphate synthase (GPPS). In some embodiments, the MEP pathway enzyme is 1-deoxy-D-xylulose 5-phosphate synthase (DXS), 1-deoxy-D-xylulose 5-phosphate reductoisomerase (DXR); 2-C-methyl-D-erythritol 4-phosphate cytidylyltransferase (CMS); 4-diphosphocytidyl-2-C-methyl-D-erythritol kinase (CMK); 2-C-methyl-D-erythritol 2,4-cyclodiphosphate synthase (MECS); 4-hydroxy-3-methyl-but-2-enyl pyrophosphate synthase (HDS); 4-hydroxy-3-methyl-but-2-enyl pyrophosphate reductase (HDR); isopentenyl pyrophosphate isomerase (IDI), or geranyl pyrophosphate synthase (GPPS). In some embodiments, the MVA pathway enzyme is mevalonate 3-phosphate-5-kinase, isopentenyl-5-phosphate kinase, mevalonate-5-phosphate decarboxylase, or mevalonate-5-kinase. In some embodiments, the increased production of GPP results in increased production of the cannabinoids described herein, e.g., CBCA, CBDA, THCA, CBCOA, CBDOA, THCOA, CBCVA, CBDVA, THCVA, CBC, CBD, and/or THC, by the non-natural flavin-dependent oxidase described herein. In some embodiments, the increased production of GPP results in increased production of CBCA, THCA, CBCOA, CBCVA, and/or CBC, CBD, by the non-natural flavin-dependent oxidase described herein.

In some embodiments, the engineered cell produces GPP from an alternative non-MEP, non-MVA geranyl pyrophosphate pathway. In some embodiments, GPP is produced from a precursor selected from isoprenol, prenol, and geraniol. In some embodiments, the engineered cell expresses an exogenous or overexpresses an exogenous or endogenous gene that encodes any one of the enzymes in a non-MVA, non-MEP pathways, thereby increasing the production of GPP. In some embodiments, the non-MVA, non-MEP pathway enzyme is alcohol kinase, alcohol diphosphokinase, phosphate kinase, isopentenyl diphosphate isomerase, or geranyl pyrophosphate synthase (GPPS). In some embodiments, the increased production of GPP results in increased production of the cannabinoids described herein, e.g., CBCA, CBDA, THCA, CBCOA, CBDOA, THCOA, CBCVA, CBDVA, THCVA, CBC, CBD, and/or THC, by the non-natural flavin-dependent oxidase described herein. In some embodiments, the increased production of GPP results in increased production of CBCA, THCA, CBCOA, CBCVA, and/or CBC, by the non-natural flavin-dependent oxidase described herein.

In some embodiments, the engineered cell an exogenous or overexpresses an exogenous or endogenous GPP synthase. Non-limiting examples of GPP synthases include E. coli IspA (NP_414955), C. glutamicum IdsA (WP_011014931.1), and the enzymes listed in Table 2.

TABLE 2
Exemplary GPP Synthases.

| | GenBank | | GenBank |
|---|---|---|---|
| Species | Accession No. | Species | Accession No. |
| Abies grandis | AAN01133.1 and | Corynebacterium | WP_035105251.1 |
| | AAN01134.1 | camporealensis | |
| Corynebacterium crudilactis | WP_074025495.1 | Corynebacterium | WP_005328932.1 |
| | | tuberculostearicum | |
| Corynebacterium | WP_096457048.1 | Corynebacterium | WP_005324491.1 |
| glutamicum | | pseudogenitalium | |
| Corynebacterium deserti | WP_053545301.1 | Corynebacterium | WP_083985528.1 |
| | | testudinoris | |
| Corynebacterium callunae | WP_015651699.1 | Corynebacterium stationis | WP_066793135.1 |
| Corynebacterium efficiens | WP_006768068.1 | Corynebacterium sp. J010B- | WP_105324112.1 |
| | | 136 | |
| Corynebacterium sp. | WP_080794061.1 | Corynebacterium sp. CCUG | WP_123047545.1 |
| Marseille-P2417 | | 69366 | |
| Corynebacterium | WP_040086238.1 | Corynebacterium sp. | WP_023030480.1 |
| humireducens | | KPL1818 | |
| Corynebacterium | WP_015401326.1 | Corynebacterium accolens | WP_005283903.1 |
| halotolerans | | | |
| Corynebacterium marinum | WP_042621772.1 | Corynebacterium | WP_126319428.1 |
| | | segmentosum | |
| Corynebacterium singulare | WP_042531577.1 | Corynebacterium | WP_121911356.1 |
| | | macginleyi | |
| Corynebacterium | WP_115022907.1 | Pseudomonas aeruginosa | SQG59150.1 |
| minutissimum | | | |
| Corynebacterium pollutisoli | WP_143337494.1 | Streptococcus thermophilus | VDG63248.1 |
| Corynebacterium | WP_018297093.1 | Nocardia vermiculata | WP_084473733.1 |
| lubricantis | | | |
| Corynebacterium | WP_092284621.1 | Rhodococcus sp. 1168 | WP_088945631.1 |
| spheniscorum | | | |
| Corynebacterium | WP_018020857.1 | Clostridium paraputrificum | WP_113570111.1 |
| doosanense | | | |
| Corynebacterium flavescens | WP_075731219.1 | Nocardia cyriacigeorgica | WP_036535265.1 |
| Corynebacterium | WP_143334899.1 | Nocardia concava | WP_040806894.1 |
| aurimucosum | | | |
| Corynebacterium | WP_003845210.1 | Rhodococcus yunnanensis | WP_072806331.1 |
| ammoniagenes | | | |
| Corynebacterium | WP_086587718.1 | | |
| kefirresidentii | | | |

In some embodiments, the disclosure provides a composition comprising the non-natural flavin-dependent oxidase described herein and one or more of the OLS described herein, the OAC described herein, and the GPP pathway enzyme described herein. In some embodiments, the disclosure provides an engineered cell comprising the non-natural flavin-dependent oxidase described herein and one or more of the OLS described herein, the OAC described herein, and the GPP pathway enzyme described herein. In some embodiments, the disclosure provides one or more polynucleotides comprising one or more nucleic acid sequences encoding the non-natural flavin-dependent oxidase described herein and one or more of the OLS described herein, the OAC described herein, and the GPP pathway enzyme described herein. In some embodiments, the GPP pathway enzyme comprises geranyl pyrophosphate (GPP) synthase, farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, or a combination thereof. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDOA, CBC, CBD, and/or THC. In some embodiments, the engineered cell produces CBCA, THCA, CBCOA, CBCVA, and/or CBC.

### Prenyltransferase

In some embodiments, the engineered cell of the present disclosure further comprises a prenyltransferase.

In general, the conversion of OA+GPP to CBGA (and the analogous conversions of OSA+GPP to CBGOA and DA+GPP to CBGVA) is performed by a prenyltransferase. In C. sativa, prenyltransferase is a transmembrane protein belonging to the UbiA superfamily of membrane proteins. Other prenyltransferases, e.g., aromatic prenyltransferases such as NphB from Streptomyces, which are non-transmembrane and soluble, can also catalyze conversion of OA to CBGA, OSA to CBGOA, and/or DA to CBGVA.

In some embodiments, the prenyltransferase is a natural prenyltransferase, e.g., wild-type prenyltransferase. In some embodiments, the prenyltransferase is a non-natural prenyltransferase. In some embodiments, the prenyltransferase comprises one or more amino acid substitutions relative to a wild-type prenyltransferase. In some embodiments, the one or more amino acid substitutions in the non-natural prenyltransferase increases the activity of the prenyltransferase as compared to a wild-type prenyltransferase.

In some embodiments, the prenyltransferase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:9. In some embodiments, the prenyltransferase is a non-natural prenyltransferase comprising at least 1, at least 2, at least 3, at least 4, at least 5, at least 6, at least 7, at least 8, at least 9, or at least 10 amino acid variations at positions corresponding to SEQ ID NO:9.

Although the amino acid positions of prenyltransferase described herein are with reference to the corresponding amino acid sequence of SEQ ID NO:9, it is understood that the amino acid sequence of a non-natural prenyltransferase can include an amino acid variation at an equivalent position corresponding to a variant of SEQ ID NO:9. One of the skill in the art would understand that alignment methods can be used to align variations of SEQ ID NO:9 to identify the position in the prenyltransferase variant that corresponds to a position in SEQ ID NO:9. In some embodiments, SEQ ID NO:9 corresponds to the amino acid sequence of Streptomyces antibioticus AQJ23_4042 prenyltransferase.

In some embodiments, the prenyltransferase comprises an amino acid substitutions at position V45, F121, T124, Q159, M160, Y173, S212, V213, A230, T267, Y286, Q293, R294, L296, F300, or a combination thereof, wherein the position corresponds to SEQ ID NO:9. In some embodiments, the prenyltransferase comprises two or more amino acid substitutions at positions V45, F121, T124, Q159, M160, Y173, S212, V213, A230, T267, Y286, Q293, R294, L296, F300, or a combination thereof. In some embodiments, the prenyltransferase comprises two or more amino acid substitutions at positions V45, F121, T124, Q159, M160, Y173, S212, V213, A230, T267, Y286, Q293, R294, L296, F300, or a combination thereof. Prenyltransferase and non-natural variants thereof are further discussed, e.g., in WO2019/173770 and WO2021/046367.

In some embodiments, the amino acid substitution is selected from V451, V45T, F121V, T124K, T124L, Q159S, M160L, M160S, Y173D, Y173K, Y173P, Y173Q, S212H, A230S, T267P, Y286V, Q293H, R294K, L296K, L296L, L296M, L296Q, F300Y, and a combination thereof.

In some embodiments, the prenyltransferase comprising an amino acid substitution as described herein is capable of a greater rate of formation of CBGA from GPP and OA, CBGOA from GPP and OSA, and/or CBGVA from GPP and DA as compared with wild-type prenyltransferase.

In some embodiments, the disclosure provides a composition comprising the non-natural flavin-dependent oxidase described herein and one or more of the OLS described herein, the OAC described herein, the GPP pathway enzyme described herein, and the prenyltransferase described herein. In some embodiments, the disclosure provides an engineered cell comprising the non-natural flavin-dependent oxidase described herein and one or more of the OLS described herein, the OAC described herein, the GPP pathway enzyme described herein, and the prenyltransferase described herein. In some embodiments, the disclosure provides one or more polynucleotides comprising one or more nucleic acid sequences encoding the non-natural flavin-dependent oxidase described herein and one or more of the OLS described herein, the OAC described herein, the GPP pathway enzyme described herein, and the prenyltransferase described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDOA, CBC, CBD, and/or THC. In some embodiments, the engineered cell produces CBCA, THCA, CBCOA, CBCVA, and/or CBC.

### Additional Strain Modifications

In some embodiments, the engineered cell of the disclosure further comprises a modification that facilitates the production of the cannabinoids described herein, e.g., CBCA, CBDA, THCA, CBCOA, CBDOA, THCOA, CBCVA, CBDVA, THCVA, CBC, CBD, and/or THC. In some embodiments, the modification increases production of a cannabinoid in the engineered cell compared with a cell not comprising the modification. In some embodiments, the modification increases efflux of a cannabinoid in the engineered cell compared with a cell not comprising the modification. In some embodiments, the cannabinoid is CBCA, THCA, CBCOA, CBCVA, and/or CBC. In some embodiments, the modification comprises expressing or upregulating the expression of an endogenous gene that facilitates production of a cannabinoid. In some embodiments, the modification comprises introducing and/or overexpression an exogenous and/or heterologous gene that facilitates production of a cannabinoid. In some embodiments, the modification comprises downregulating, disrupting, or deleting an endogenous gene that hinders production of a cannabinoid. Expression and/or overexpression of endogenous and exogenous genes, and downregulation, disruption and/or deletion of endogenous genes are described in embodiments herein.

In some embodiments, the engineered cell of the disclosure comprises one or more of the following modifications:
i) express one or more exogenous nucleic acid sequences or overexpress one or more endogenous genes encoding a protein having an ABC transporter permease activity;
ii) express one or more exogenous nucleic acid sequences or overexpress one or more endogenous genes encoding a protein having an ABC transporter ATP-binding protein activity;
iii) express one or more exogenous nucleic acids sequences or overexpress one or more endogenous genes selected from blc, ydhC, ydhG, or a homolog thereof;
iv) express one or more exogenous nucleic acids sequences or overexpress one or more endogenous genes selected from mlaD, mlaE, mlaF, or a homolog thereof;
v) express one or more exogenous nucleic acid sequences or overexpress one or more endogenous genes encoding a protein having a siderophore receptor protein activity or overexpress one or more endogenous genes encoding a protein having a siderophore receptor protein activity;
vi) comprise a disruption of or downregulation in the expression of a regulator of expression of one or more endogenous genes encoding a protein having an ABC transporter permease activity, a protein having an ABC transporter ATP-binding protein activity, a blc gene, a ybhG protein, a ydhC protein, a mlaD protein, mlaE protein, mlaF protein, or a protein having a siderophore receptor protein activity;
vii) express one or more exogenous nucleic acids sequences or overexpress one or more endogenous genes encoding a multi-domain protein having acetyl-CoA carboxylase activity (MD-ACC);
viii) express one or more exogenous nucleic acids sequences or overexpress one or more endogenous genes encoding acetyl-CoA carboxyltransferase subunit α, biotin carboxyl carrier protein, biotin carboxylase, or acetyl-CoA carboxyltransferase subunit β, or express one or more exogenous nucleic acids or overexpress one or more endogenous genes encoding acetyl-CoA carboxyltransferase, biotin carboxyl carrier protein, or biotin carboxylase activities;
ix) disruption of or downregulation in the expression of an endogenous gene encoding a protein having (acyl-carrier-protein) S-malonyltransferase activity, an endogenous gene encoding a protein having 3-hydroxypalmitoyl-(acyl-carrier-protein) dehydratase activity, or both;
x) express an exogenous nucleic acid sequence or overexpress an endogenous gene encoding a protein having fatty acyl-CoA ligase activity, or both;
xi) disruption of or downregulation in the expression of at least one endogenous gene encoding a protein having acyl-CoA dehydrogenase activity or enoyl-CoA hydratase activity;
xii) comprise a disruption of or downregulation in the expression of at least one endogenous gene encoding a protein having acyl-CoA esterase/thioesterase activity;
xiii) comprise a disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway;
xiv) express an exogenous nucleic acid sequence or overexpress an endogenous gene encoding a protein having geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, prenol kinase activity, prenol diphosphokinase activity, isoprenol kinase activity, isoprenol diphosphokinase activity, dimethylallyl phosphate kinase activity, isopentenyl phosphate kinase activity, or isopentenyl diphosphate isomerase activity;
xv) express an exogenous nucleic acid sequence or overexpress an endogenous gene encoding a protein having GPP synthase activity;
xvi) express an exogenous nucleic acid sequence encoding an olivetol synthase;
xvii) express an exogenous nucleic acid sequence encoding an olivetolic acid cyclase;
xviii) express an exogenous nucleic acid sequence encoding a prenyltransferase;
xix) express one or more exogenous nucleic acid sequences or overexpressing one or more endogenous genes encoding one or more enzymes of MVA pathway, MEP pathway, or a non-MVA, non-MEP pathway;
xx) express an exogenous nucleic acid sequence or overexpress an endogenous gene encoding a biotin-(acetyl-CoA carboxylase) ligase;
xxi) express an exogenous nucleic acid sequence or overexpress an endogenous gene encoding a isopentenyl-diphosphate delta-isomerase;
xxii) express an exogenous nucleic acid sequence or overexpress an endogenous gene encoding a hydroxyethylthiazole kinase or both;
xxiii) express an exogenous nucleic acid sequence or overexpress an endogenous gene encoding a Type III pantothenate kinase; and
xxiv) comprise a disruption of or downregulation in the expression of at least one endogenous gene encoding a phosphatase selected from the group consisting of ADP-sugar pyrophosphatase, dihydroneopterin triphosphate diphosphatase, pyrimidine deoxynucleotide diphosphatase, pyrimidine pyrophosphate phosphatase, and Nudix hydrolase.

In some embodiments, the disclosure provides an engineered cell comprising the non-natural flavin-dependent oxidase described herein and one or more of the OLS described herein, the OAC described herein, the GPP pathway enzyme described herein, the prenyltransferase described herein, and an additional modification described herein. In some embodiments, the disclosure provides one or more polynucleotides comprising one or more nucleic acid sequences encoding the non-natural flavin-dependent oxidase described herein and one or more of the OLS described herein, the OAC described herein, the GPP pathway enzyme described herein, the prenyltransferase described herein, and an additional modification described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDOA, CBC, CBD, and/or THC. In some embodiments, the engineered cell produces CBCA, THCA, CBCOA, CBCVA, and/or CBC.

### Host Cells

A variety of microorganisms may be suitable as the engineered cell described herein. Such organisms include both prokaryotic and eukaryotic organisms including, but not limited to, bacteria, including archaea and eubacteria, and eukaryotes, including yeast, plant, and insect. Nonlimiting examples of suitable microbial hosts for the bio-production of a cannabinoid include, but are not limited to, any Gram negative organisms, more particularly a member of the family Enterobacteriaceae, such as E. coli, or Oligotropha carboxidovorans, or a Pseudomononas sp.; any Gram positive microorganism, for example Bacillus subtilis, Lactobaccilus sp. or Lactococcus sp.; a yeast, for example Saccharomyces cerevisiae, Pichia pastoris or Pichia stipitis; and other groups or microbial species. In some embodiments, the microbial host is a member of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, or Saccharomyces. In some embodiments, the microbial host is Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis or Saccharomyces cerevisiae.

Further exemplary species are reported in U.S. Pat. No. 9,657,316 and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In some embodiments, the engineered cell is a bacterial cell or a fungal cell. In some embodiments, the engineered cell is a bacterial cell. In some embodiments, the engineered cell is a yeast cell. In some embodiments, the engineered cell is an algal cell. In some embodiments, the engineered cell is a cyanobacterial cell. In some embodiments, the bacteria is Escherichia, Corynebacterium, Bacillus, Ralstonia, Zymomonas, or Staphylococcus. In some embodiments, the bacterial cell is an Escherichia coli cell.

In some embodiments, the engineered cell is an organism selected from Acinetobacter baumannii Naval-82, Acinetobacter sp. ADP 1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chlorojlexus aggregans DSM 9485, Chlorojlexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bern, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobiurn denitrificans ATCC 51888, Hyphomicrobiurn zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobiurn loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacteriurn extorquens, Methylobacteriurn extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobiurn meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, and Zea mays.

Algae that can be engineered for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta.

Microalgae (single-celled algae) produce natural oils that can contain the synthesized cannabinoids. Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditiana. Dunaliella salina. Dunaliella tertiolecta, Chlorella vulgaris, Chlorella variabilis, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania. Eremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus. Platymonas, Pleurochrsis, Pleurococcus, Prototheca, Pseudochlorella, Pyramimonas, Pvrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scvtonema, Spirulina, Stanieria, Starria, Stigonema, Symploca, Synechococcus, Svnechocystis, Tolipothrix, Trichodesmium. Tychonema, and Xenococcus species.

The host cell may be genetically modified for a recombinant production system, e.g., to produce CBCA, CBDA, THCA, CBCOA, CBDOA, THCOA, CBCVA, CBDVA, THCVA, CBC, CBD, and/or THC as described herein. In some embodiments, the host cell is genetically modified to produce CBCA, THCA, CBCOA, CBCVA, and/or CBC as described herein. The mode of gene transfer technology may be by electroporation, conjugation, transduction or natural transformation as described herein.

To genetically modify a host cell of the disclosure, one or more heterologous nucleic acids disclosed herein is introduced stably or transiently into a host cell, using established techniques. Such techniques may include, but are not limited to, electroporation, calcium phosphate precipitation, DEAE-dextran mediated transfection, liposome-mediated transfection, particle bombardment, and the like. For stable transformation, a heterologous nucleic acid will generally further include a selectable marker, e.g., any of several well-known selectable markers such as neomycin resistance, ampicillin resistance, tetracycline resistance, chloramphenicol resistance, kanamycin resistance, hygromycin resistance, G418 resistance, bleomycin resistance, zeocin resistance, and the like. A broad range of plasmids and drug resistance markers are available and described in embodiments herein. The cloning vectors are tailored to the host organisms based on the nature of antibiotic resistance markers that can function in that host cell. In some embodiments, the host cell is genetically modified using CRISPR/Cas9 to produce the engineered cell of the disclosure.

### Fermentation

In some embodiments, the disclosure provides a method of producing a cannabinoid or precursor thereof, e.g., CBCA, CBDA, THCA, CBCOA, CBDOA, THCOA, CBCVA, CBDVA, THCVA, CBC, CBD, and/or THC, as described herein, comprising culturing an engineered cell provided herein to provide the cannabinoid. In some embodiments, the method further comprises recovering the cannabinoid, e.g., CBCA, CBDA, THCA, CBCOA, CBDOA, THCOA, CBCVA, CBDVA, THCVA, CBC, CBD, and/or THC from the cell, cell extract, culture medium, whole culture, or a combination thereof. In some embodiments, the cannabinoid is CBCA, THCA, CBCOA, CBCVA, CBC, or a combination thereof.

In some embodiments, the culture medium of the engineered cells further comprises at least one carbon source. In embodiments where the cells are heterotrophic cells, the culture medium comprises at least one carbon source that is also an energy source, also known as a “feed molecule.” In some embodiments, the culture medium comprises one, two, three, or more carbon sources that are not primary energy sources. Non-limiting examples of feed molecules that can be included in the culture medium include acetate, malonate, oxaloacetate, aspartate, glutamate, beta-alanine, alpha-alanine, butyrate, hexanoate, hexanol, prenol, isoprenol, and geraniol. Further examples of compounds that can be provided in the culture medium include, without limitation, biotin, thiamine, pantotheine, and 4-phosphopantetheine.

In some embodiments, the culture medium comprises acetate. In some embodiments, the culture medium comprises acetate and hexanoate. In some embodiments, the culture medium comprises malonate and hexanoate. In some embodiments, the culture medium comprises prenol, isoprenol, and/or geraniol. In some embodiments, the culture medium comprises aspartate, hexanoate, prenol, isoprenol, and/or geraniol.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used herein, culture medium, or simply “medium” as it relates to the growth source, refers to the starting medium, which may be in a solid or liquid form. “Cultured medium” as used herein refers to medium (e.g. liquid medium) containing microbes that have been fermentatively grown and can include other cellular biomass. The medium generally includes one or more carbon sources, nitrogen sources, inorganic salts, vitamins and/or trace elements. “Whole culture” as used herein refers to cultured cells plus the culture medium in which they are cultured. “Cell extract” as used herein refers to a lysate of the cultured cells, which may include the culture medium and which may be crude (unpurified), purified or partially purified. Methods of purifying cell lysates are known to the skilled artisan and described in embodiments herein.

Exemplary carbon sources include sugar carbons such as sucrose, glucose, galactose, fructose, mannose, isomaltose, xylose, maltose, arabinose, cellobiose and 3-, 4-, or 5-oligomers thereof. Other carbon sources include carbon sources such as methanol, ethanol, glycerol, formate and fatty acids. Still other carbon sources include carbon sources from gas such as synthesis gas, waste gas, methane, CO, CO₂ and any mixture of CO, CO₂ with Hz. Other carbon sources can include renewal feedstocks and biomass. Exemplary renewal feedstocks include cellulosic biomass, hemicellulosic biomass and lignin feedstocks.

In some embodiments, the engineered cell is sustained, cultured, or fermented under aerobic, microaerobic, anaerobic or substantially anaerobic conditions. Exemplary aerobic, microaerobic, and anaerobic conditions have been described previously and are known in the art. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation, or higher. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N2/CO₂ mixture or other suitable non-oxygen gas or gases. Exemplary anaerobic conditions for fermentation processes are described, for example, in U.S. Patent Publication No. 2009/0047719. Any of these conditions can be employed with the microbial organisms described herein as well as other anaerobic conditions known in the field. The culture conditions can include, for example, liquid culture procedures as well as fermentation and other large scale culture procedures.

The culture conditions can be scaled up and grown continuously for manufacturing the cannabinoid products described herein. Exemplary growth procedures include, for example, fed-batch fermentation and batch separation; fed-batch fermentation and continuous separation, or continuous fermentation and continuous separation. Fermentation procedures can be particularly useful for the biosynthetic production of commercial quantities of cannabinoids, e.g., CBCA, CBDA, THCA, CBCOA, CBDOA, CBCVA, CBDVA, THCVA, THCOA, CBC, CBD, and/or THC. In some embodiments, the cannabinoid is CBCA, CBCOA, CBCVA, CBC, or a combination thereof. Generally, and as with non-continuous culture procedures, the continuous and/or near-continuous production of cannabinoid product can include culturing a cannabinoid-producing organism with sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are known to the skilled artisan. Briefly, fermentation for the biosynthetic production of a cannabinoid, e.g., CBCA, CBDA, THCA, CBCOA, CBDOA, THCOA, CBCVA, CBDVA, THCVA, CBC, CBD, and/or THC, can be utilized in, for example, fed-batch fermentation and batch separation; fed-batch fermentation and continuous separation, or continuous fermentation and continuous separation. Examples of batch and continuous fermentation procedures are known in the field. Typically, cells are grown at a temperature in the range of about 25° C. to about 40° C. in an appropriate medium, as well as up to 70° C. for thermophilic microorganisms. In some embodiments, the cannabinoid is CBCA, CBCOA, CBCVA, CBC, or a combination thereof.

The culture medium at the start of fermentation may have a pH of about 4 to about 7. The pH may be less than 11, less than 10, less than 9, or less than 8. In some embodiments, the pH is at least 2, at least 3, at least 4, at least 5, at least 6, or at least 7. In some embodiments, the pH of the medium is about 6 to about 9.5; 6 to about 9, about 6 to 8 or about 8 to 9.

In some embodiments, upon completion of the cultivation period, the fermenter contents are passed through a cell separation unit, for example, a centrifuge, filtration unit, and the like, to remove cells and cell debris. In embodiments where the desired product is expressed intracellularly, the cells are lysed or disrupted enzymatically or chemically prior to or after separation of cells from the fermentation broth, as desired, in order to release additional product. The fermentation broth can be transferred to a product separations unit. Isolation of product can be performed by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, methylene chloride, chloroform, benzene, pentane, hexane, heptane, petroleum ether, methyl tertiary butyl ether (MTBE), and the like) to provide an organic solution of the product, if appropriate, standard distillation methods, and the like, depending on the chemical characteristics of the product of the fermentation process.

Suitable purification and/or assays to test a cannabinoid, e.g., CBCA, CBDA, THCA, CBCOA, CBDOA, THCOA, CBCVA, CBDVA, THCVA, CBC, CBD, and/or THC, produced by the methods herein can be performed using known methods. In some embodiments, the cannabinoid is CBCA, CBCOA, CBCVA, CBC, or a combination thereof. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al. (2005), Biotechnol. Bioeng. 90:775-779), or other suitable assay and detection methods well known in the art. The individual enzyme or protein activities from the exogenous DNA sequences can also be assayed using methods known in the art.

The cannabinoids produced using methods described herein can be separated from other components in the culture using a variety of methods well known in the art. Such separation methods include, for example, extraction procedures as well as methods that include liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. For example, the amount of cannabinoid or other product(s), including a polyketide, produced in a bio-production media generally can be determined using any of methods such as, for example, high performance liquid chromatography (HPLC), gas chromatography (GC), GC/Mass Spectroscopy (MS), or spectrometry.

In some embodiments, the cell extract or cell culture medium described herein comprises a cannabinoid. In some embodiments, the cannabinoid is cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol type, or a combination thereof. In some embodiments, the cannabinoid is cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C4 (THCA-C4), Δ⁹-tetrahydrocannabinol-C4 (THC-C4), Δ⁹-tetrahydrocannabivarinic acid (THCVA), Δ⁹-tetrahydrocannabivarin (THCV), Δ⁹-tetrahydrocannabiorcolic acid (THCA-C1), Δ⁹-tetrahydrocannabiorcol (THC-C1), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol, 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran, 10-oxo-delta-6a-tetrahydrocannabinol (OTHC), Δ⁹-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-Δ⁹-tetrahydrocannabinol (triOH-THC), or a combination thereof.

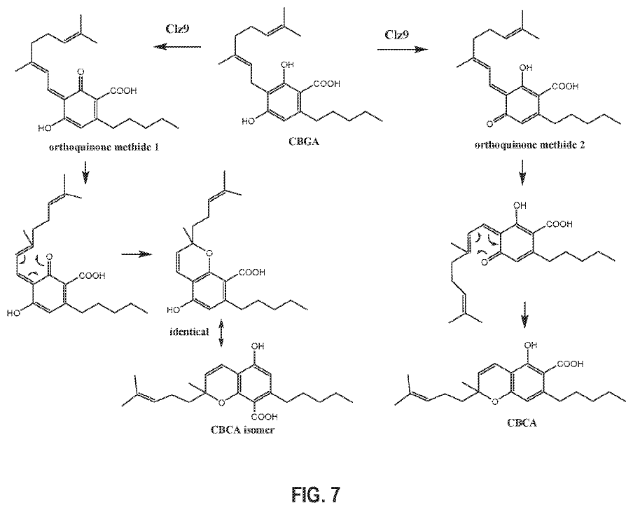

In some embodiments, the disclosure provides a cell extract or cell culture medium comprising cannabigerolic acid (CBGA), cannabichromenic acid (CBCA), cannabidiolic acid (CBDA), tetrahydrocannabinolic acid (THCA), cannabigerol (CBG), cannabichromene (CBC), cannabidiol (CBD), tetrahydrocannabinol (THC), cannabigerorcinic acid (CBGOA), cannabiorcichromenic acid (CBCOA), cannabidiorsellinic acid (CBDOA), tetraydrocannabiorcolic acid (THCOA), cannabigerivarinic acid (CBGVA), cannabichromevarinic acid (CBCVA), cannabidivarinic acid (CBDVA), tetrahydrocannabivarin acid (THCVA), an isomer, analog or derivative thereof, or a combination thereof derived from the engineered cell described herein. In some embodiments, the disclosure provides a cell extract or cell culture medium comprising CBGA, CBCA, THCA, CBG, CBC, CBGOA, CBCOA, CBGVA, CBCVA, an isomer, analog or derivative thereof, or a combination thereof derived from the engineered cell described herein. Isomers, analogs, and derivatives of the cannabinoids described herein are known to one of ordinary skill in the art and include, e.g., the CBCA isomers shown in FIG. 7. In some embodiments, a derivative of a cannabinoid described herein, e.g., CBGA, CBCA, CBDA, THCA, CBGOA, CBCOA, CBDOA, THCOA, CBGVA, CBCVA, CBDVA, and/or THCVA, is a decarboxylated form of the cannabinoid.

In some embodiments, the disclosure provides a novel compound of Formula I:

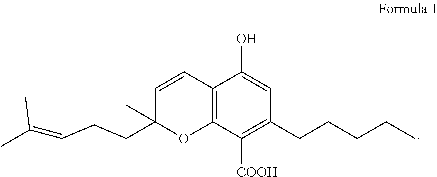 [SMILES: CCCCCc1cc(O)c2c(c1C(=O)O)OC(C)(CCC=C(C)C)C=C2]

In some embodiments, the disclosure provides a method of making CBC, comprising converting the compound of Formula I into CBC. In some embodiments, the disclosure provides a method of making CBC, comprising contacting CBGA with a flavin-dependent oxidase described herein to form a compound of Formula I; and converting the compound of Formula I into CBC. In some embodiments, the compound of Formula I converts into CBC by decarboxylation.

### Method of Making or Isolating

In some embodiments, the disclosure provides a method of making a cannabinoid selected from CBCA, CBC, CBCOA, CBCVA, CBDA, CBD, CBDOA, CBDVA, THCA, THC, THCOA, THCVA, an isomer, analog or derivative thereof, or a combination thereof, comprising culturing the engineered cell as described herein, or isolating the cannabinoid from the cell extract or cell culture medium as described herein. In some embodiments, the cannabinoid is CBCA, CBC, CBCOA, CBCVA, THCA, an isomer, analog or derivative thereof, or a combination thereof.

In some embodiments, the disclosure provides a method of making CBCA, CBC, CBCOA, CBCVA, CBDA, CBD, CBDOA, CBDVA, THCA, THC, THCOA, THCVA, an isomer, analog or derivative thereof, or a combination thereof, comprising culturing the engineered cell comprising the non-natural flavin-dependent oxidase described herein, the polynucleotide described herein comprising the nucleic acid sequence encoding the non-natural flavin-dependent oxidase, the expression construct comprising the polynucleotide, or a combination thereof. In embodiments, the method makes CBCA, CBC, CBCOA, CBCVA, THCA, an isomer, analog or derivative thereof, or a combination thereof. In some embodiments, the disclosure provides a method of isolating CBCA, CBC, CBCOA, CBCVA, CBDA, CBD, CBDOA, CBDVA, THCA, THC, THCOA, THCVA an isomer, analog or derivative thereof, or a combination thereof, from the cell extract or cell culture medium of the engineered cell. In embodiments, the method isolates CBCA, CBC, CBCOA, CBCVA, THCA, an isomer, analog or derivative thereof, or a combination thereof.

Methods of culturing cells, e.g., the engineered cell of the disclosure, are provided herein. Methods of isolating a cannabinoid, e.g., CBCA, CBC, CBCOA, CBCVA, CBDA, CBD, CBDOA, CBDVA, THCA, THC, THCOA, THCVA, an isomer, analog or derivative thereof, are also provided herein. In some embodiments, the cannabinoid is CBCA, CBC, CBCOA, CBCVA, THCA, an isomer, analog or derivative thereof, or a combination thereof. In some embodiments, the isolating comprises liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, ultrafiltration, or a combination thereof.

In some embodiments, the disclosure provides a method of making CBCA, CBDA, THCA, or an isomer, analog or derivative thereof, or a combination thereof, comprising contacting CBGA with the non-natural flavin-dependent oxidase described herein. In some embodiments, the disclosure provides a method of making CBCA, CBDA, THCA, or an isomer, analog or derivative thereof, or a combination thereof, comprising contacting CBGA with a flavin-dependent oxidase comprising any of SEQ ID NOs:1-6. In some embodiments, the method makes CBCA, THCA, or an isomer, analog or derivative thereof, or a combination thereof. In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:4. In some embodiments, the flavin-dependent oxidase comprises SEQ ID NO:5. In some embodiments, the flavin-dependent oxidase comprises SEQ ID NO:6. In some embodiments, the flavin-dependent oxidase comprise a polypeptide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:1-6. In some embodiments, the flavin-dependent oxidase does not comprise a disulfide bond. In some embodiments, the flavin-dependent oxidase is not glycosylated. In some embodiments, the flavin-dependent oxidase has substantially the same catalytic activity at about pH 5 to about pH 8.

In some embodiments, the disclosure provides a method of making CBCA, CBDA, THCA, or an isomer, analog or derivative thereof, comprising contacting CBGA with EncM. In some embodiments, the EncM is a wild-type EncM. In some embodiments, the EncM comprises a modification, e.g., an amino acid variation and/or a tag as described herein. In some embodiments, the disclosure provides a method of making CBCA, CBDA, THCA, or an isomer, analog or derivative thereof, comprising contacting CBGA with Clz9. In some embodiments, the Clz9 is a wild-type Clz9. In some embodiments, the Clz9 comprises a modification, e.g., an amino acid variation and/or a tag as described herein. In some embodiments, the method makes CBCA, THCA, or an isomer, analog or derivative thereof. In some embodiments, the method makes CBCA or an isomer, analog or derivative thereof.

In some embodiments, the contacting occurs at about pH 4 to about pH 9, about pH 4.5 to about pH 8.5, about pH 5 to about pH 8, about pH 5.5 to about pH 7.5, or about pH 5 to about pH 7. In some embodiments, the method is performed in an in vitro reaction medium, e.g., an aqueous reaction medium. In some embodiments, the reaction medium further comprises a buffer, a salt, a surfactant, or a combination thereof. In some embodiments, the surfactant is about 0.005% (v/v) to about 5% (v/v) of the in vitro reaction medium. In some embodiments, the surfactant is about 0.01% (v/v) to about 1% (v/v) of the in vitro reaction medium. In some embodiments, the surfactant is about 0.05% (v/v) to about 0.5% (v/v) of the in vitro reaction medium. In some embodiments, the surfactant is about 0.08% (v/v) to about 0.2% (v/v) of the in vitro reaction medium. In some embodiments, the surfactant is a nonionic surfactant. Non-limiting examples of nonionic surfactants include TRITON™ X-100, TWEEN®, IGEPAL® CA-630, NONIDET™ P-40, and the like. In some embodiments, the surfactant is 2-[4-(2,4,4-trimethylpentan-2-yl)phenoxy]ethanol (also known as TRITON™ X-100). In some embodiments, the in vitro reaction medium comprises about 0.1% (v/v) 2-[4-(2,4,4-trimethylpentan-2-yl)phenoxy]ethanol.

In some embodiments, the disclosure provides a method of making CBCOA, CBDOA, THCOA, or an isomer, analog or derivative thereof, or a combination thereof, comprising contacting CBGOA with the non-natural flavin-dependent oxidase described herein. In some embodiments, the disclosure provides a method of making CBCOA, CBDOA, THCOA, or an isomer, analog or derivative thereof, or a combination thereof, comprising contacting CBGOA with a flavin-dependent oxidase comprising any of SEQ ID NOs:1-6. In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:4. In some embodiments, the flavin-dependent oxidase comprises SEQ ID NO:5. In some embodiments, the flavin-dependent oxidase comprises SEQ ID NO:6. In some embodiments, the flavin-dependent oxidase comprise a polypeptide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:1-6. In some embodiments, the method makes CBCOA or an isomer, analog or derivative thereof.

In some embodiments, the disclosure provides a method of making CBCOA, CBDOA, THCOA, or an isomer, analog or derivative thereof, or a combination thereof, comprising contacting CBGOA with EncM. In some embodiments, the EncM is a wild-type EncM. In some embodiments, the EncM comprises a modification, e.g., an amino acid variation and/or a tag as described herein. In some embodiments, the disclosure provides a method of making CBCOA, CBDOA, THCOA, or an isomer, analog or derivative thereof, or a combination thereof, comprising contacting CBGOA with Clz9. In some embodiments, the Clz9 is a wild-type Clz9. In some embodiments, the Clz9 comprises a modification, e.g., an amino acid variation and/or a tag as described herein. In some embodiments, the method makes CBCOA or an isomer, analog or derivative thereof.

In some embodiments, the contacting occurs at about pH 4 to about pH 9, about pH 4.5 to about pH 8.5, about pH 5 to about pH 8, about pH 5.5 to about pH 7.5, or about pH 5 to about pH 7. In some embodiments, the method is performed in an in vitro reaction medium, e.g., an aqueous reaction medium. In some embodiments, the reaction medium further comprises a buffer, a salt, a surfactant, or a combination thereof. In some embodiments, the surfactant is about 0.005% (v/v) to about 5% (v/v) of the in vitro reaction medium. In some embodiments, the surfactant is about 0.01% (v/v) to about 1% (v/v) of the in vitro reaction medium. In some embodiments, the surfactant is about 0.05% (v/v) to about 0.5% (v/v) of the in vitro reaction medium. In some embodiments, the surfactant is about 0.08% (v/v) to about 0.2% (v/v) of the in vitro reaction medium. In some embodiments, the surfactant is a nonionic surfactant. Non-limiting examples of nonionic surfactants include TRITON™ X-100, TWEEN®, IGEPAL® CA-630, NONIDET™ P-40, and the like. In some embodiments, the surfactant is 2-[4-(2,4,4-trimethylpentan-2-yl)phenoxy]ethanol (also known as TRITON™ X-100). In some embodiments, the in vitro reaction medium comprises about 0.1% (v/v) 2-[4-(2,4,4-trimethylpentan-2-yl)phenoxy]ethanol.

In some embodiments, the disclosure provides a method of making CBCVA, CBDVA, THCVA, or an isomer, analog or derivative thereof, or a combination thereof, comprising contacting CBGVA with the non-natural flavin-dependent oxidase described herein. In some embodiments, the disclosure provides a method of making CBCVA, CBDVA, THCVA, or an isomer, analog or derivative thereof, or a combination thereof, comprising contacting CBGVA with a flavin-dependent oxidase comprising any of SEQ ID NOs:1-6. In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:4. In some embodiments, the flavin-dependent oxidase comprises SEQ ID NO:5. In some embodiments, the flavin-dependent oxidase comprises SEQ ID NO:6. In some embodiments, the flavin-dependent oxidase comprise a polypeptide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:1-6. In some embodiments, the method makes CBCVA or an isomer, analog or derivative thereof.

In some embodiments, the disclosure provides a method of making CBCVA, CBDVA, THCVA, or an isomer, analog or derivative thereof, or a combination thereof, comprising contacting CBGVA with EncM. In some embodiments, the EncM is a wild-type EncM. In some embodiments, the EncM comprises a modification, e.g., an amino acid variation and/or a tag as described herein. In some embodiments, the disclosure provides a method of making CBCVA, CBDVA, THCVA, or an isomer, analog or derivative thereof, or a combination thereof, comprising contacting CBGVA with Clz9. In some embodiments, the Clz9 is a wild-type Clz9. In some embodiments, the Clz9 comprises a modification, e.g., an amino acid variation and/or a tag as described herein. In some embodiments, the method makes CBCVA or an isomer, analog or derivative thereof.

In some embodiments, the contacting occurs at about pH 4 to about pH 9, about pH 4.5 to about pH 8.5, about pH 5 to about pH 8, about pH 5.5 to about pH 7.5, or about pH 5 to about pH 7. In some embodiments, the method is performed in an in vitro reaction medium, e.g., an aqueous reaction medium. In some embodiments, the reaction medium further comprises a buffer, a salt, a surfactant, or a combination thereof. In some embodiments, the surfactant is about 0.005% (v/v) to about 5% (v/v) of the in vitro reaction medium. In some embodiments, the surfactant is about 0.01% (v/v) to about 1% (v/v) of the in vitro reaction medium. In some embodiments, the surfactant is about 0.05% (v/v) to about 0.5% (v/v) of the in vitro reaction medium. In some embodiments, the surfactant is about 0.08% (v/v) to about 0.2% (v/v) of the in vitro reaction medium. In some embodiments, the surfactant is a nonionic surfactant. Non-limiting examples of nonionic surfactants include TRITON™ X-100, TWEEN®, IGEPAL® CA-630, NONIDET™ P-40, and the like. In some embodiments, the surfactant is 2-[4-(2,4,4-trimethylpentan-2-yl)phenoxy]ethanol (also known as TRITON™ X-100). In some embodiments, the in vitro reaction medium comprises about 0.1% (v/v) 2-[4-(2,4,4-trimethylpentan-2-yl)phenoxy]ethanol.

In some embodiments, the disclosure provides a method of making CBC, CBD, THC, or an isomer, analog or derivative thereof, or a combination thereof, comprising contacting CBG with the non-natural flavin-dependent oxidase described herein. In some embodiments, the disclosure provides a method of making CBC, CBD, THC, or an isomer, analog or derivative thereof, or a combination thereof, comprising contacting CBG with a flavin-dependent oxidase comprising any of SEQ ID NOs:1-6. In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:4. In some embodiments, the flavin-dependent oxidase comprises SEQ ID NO:5. In some embodiments, the flavin-dependent oxidase comprises SEQ ID NO:6. In some embodiments, the flavin-dependent oxidase comprise a polypeptide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:1-6. In some embodiments, the method makes CBC or an isomer, analog or derivative thereof.

In some embodiments, the disclosure provides a method of making CBC, CBD, THC, or an isomer, analog or derivative thereof, or a combination thereof, comprising contacting CBG with EncM. In some embodiments, the EncM is a wild-type EncM. In some embodiments, the EncM comprises a modification, e.g., an amino acid variation and/or a tag as described herein. In some embodiments, the disclosure provides a method of making CBC, CBD, THC, or an isomer, analog or derivative thereof, or a combination thereof, comprising contacting CBG with Clz9. In some embodiments, the Clz9 is a wild-type Clz9. In some embodiments, the Clz9 comprises a modification, e.g., an amino acid variation and/or a tag as described herein. In some embodiments, the method makes CBC or an isomer, analog or derivative thereof.

In some embodiments, the contacting occurs at about pH 4 to about pH 9, about pH 4.5 to about pH 8.5, about pH 5 to about pH 8, about pH 5.5 to about pH 7.5, or about pH 5 to about pH 7. In some embodiments, the method is performed in an in vitro reaction medium, e.g., an aqueous reaction medium. In some embodiments, the reaction medium further comprises a buffer, a salt, a surfactant, or a combination thereof. In some embodiments, the surfactant is about 0.005% (v/v) to about 5% (v/v) of the in vitro reaction medium. In some embodiments, the surfactant is about 0.01% (v/v) to about 1% (v/v) of the in vitro reaction medium. In some embodiments, the surfactant is about 0.05% (v/v) to about 0.5% (v/v) of the in vitro reaction medium. In some embodiments, the surfactant is about 0.08% (v/v) to about 0.2% (v/v) of the in vitro reaction medium. In some embodiments, the surfactant is a nonionic surfactant. Non-limiting examples of nonionic surfactants include TRITON™ X-100, TWEEN®, IGEPAL® CA-630, NONIDET™ P-40, and the like. In some embodiments, the surfactant is 2-[4-(2,4,4-trimethylpentan-2-yl)phenoxy]ethanol (also known as TRITON™ X-100). In some embodiments, the in vitro reaction medium comprises about 0.1% (v/v) 2-[4-(2,4,4-trimethylpentan-2-yl)phenoxy]ethanol.

As discussed herein, naturally-occurring cannabinoid synthases from C. sativa do not accept CBG as a substrate. Thus, the flavin-dependent oxidases described herein that are capable of converting CBG into CBC, e.g., EncM and Clz9, advantageously expand the repertoire of cannabinoids that can be produced enzymatically by microbial host cells, e.g., bacterial cells.

In some embodiments, EncM does not comprise a disulfide bond. In some embodiments, EncM is not glycosylated. In some embodiments, EncM has substantially the same catalytic activity at about pH 5 to about pH 8. In some embodiments, Clz9 does not contain a disulfide bond. In some embodiments, Clz9 is not glycosylated. In some embodiments, Clz9 has substantially the same catalytic activity at about pH 5 to about pH 8. The advantages of non-disulfide-containing, non-glycosylated proteins that are active at a wide range of pH, including neutral pH, are further discussed herein and include, e.g., the ability to produce such proteins in large quantities and with high activity by microbial host cells using standard fermentation processes.

In some embodiments, the non-natural flavin-dependent oxidase is produced by an engineered cell. In some embodiments, the non-natural flavin-dependent oxidase is overexpressed, e.g., on an exogenous nucleic acid such as a plasmid, by an inducible or constitutive promoter, in an engineered cell. In some embodiments, the disclosure provides a method of making an isolated non-natural flavin-dependent oxidase, comprising isolating the non-natural flavin-dependent oxidase expressed in the engineered cell. Methods of culturing cells, e.g., the engineered cell of the disclosure, are provided herein. In some embodiments, the disclosure provides an isolated non-natural flavin-dependent oxidase made by the methods provided herein.

Methods of isolating proteins (e.g., the non-natural flavin-dependent oxidase) from cells are known in the art. For example, the cells can be lysed to form a crude lysate, and the crude lysate can be further purified using filtration, centrifugation, chromatography, buffer exchange, or a combination thereof. The cell lysate is considered partially purified when about 10% to about 60%, or about 20% to about 50%, or about 30% to about 50% of the total proteins in the lysate is the desired protein of interest, e.g., the non-natural flavin-dependent oxidase. A protein can also be isolated from the cell lysate as a purified protein when greater than 60%, greater than 70%, greater than 80%, greater than 90%, greater than 95%, or greater than 99% of total proteins in the lysate is the desired protein of interest, e.g., the non-natural flavin-dependent oxidase.

In some embodiments, the crude lysate comprising the non-natural flavin-dependent oxidase is capable of converting CBGA to CBCA, CBDA, THCA, or an isomer, analog or derivative thereof. In some embodiments, the CBGA is contacted with crude cell lysate comprising the non-natural flavin-dependent oxidase to form CBCA, CBDA, THCA, or an isomer, analog or derivative thereof. In some embodiments, a partially purified lysate comprising the non-natural flavin-dependent oxidase is capable of converting CBGA to CBCA, CBDA, THCA, or an isomer, analog or derivative thereof. In some embodiments, the CBGA is contacted with partially purified lysate comprising the non-natural flavin-dependent oxidase to form CBCA, CBDA, THCA, or an isomer, analog or derivative thereof. In some embodiments, a purified non-natural flavin-dependent oxidase is capable of converting CBGA to CBCA, CBDA, THCA, or an isomer, analog or derivative thereof. In some embodiments, the CBGA is contacted with purified non-natural flavin-dependent oxidase to form CBCA, CBDA, THCA, or an isomer, analog or derivative thereof. In some embodiments, the CBGA is converted to CBCA or an isomer, analog or derivative thereof. In some embodiments, the CBGA is converted to THCA or an isomer, analog or derivative thereof.

In some embodiments, the crude lysate comprising the non-natural flavin-dependent oxidase is capable of converting CBGOA to CBCOA, CBDOA, THCOA, or an isomer, analog or derivative thereof. In some embodiments, the CBGOA is contacted with crude cell lysate comprising the non-natural flavin-dependent oxidase to form CBCOA, CBDOA, THCOA, or an isomer, analog or derivative thereof. In some embodiments, a partially purified lysate comprising the non-natural flavin-dependent oxidase is capable of converting CBGOA to CBCOA, CBDOA, THCOA, or an isomer, analog or derivative thereof. In some embodiments, the CBGOA is contacted with partially purified lysate comprising the non-natural flavin-dependent oxidase to form CBCOA, CBDOA, THCOA, or an isomer, analog or derivative thereof. In some embodiments, a purified non-natural flavin-dependent oxidase is capable of converting CBGOA to CBCOA, CBDOA, THCOA, or an isomer, analog or derivative thereof. In some embodiments, the CBGOA is contacted with purified non-natural flavin-dependent oxidase to form CBCOA, CBDOA, THCOA, or an isomer, analog or derivative thereof. In some embodiments, the CBGOA is converted to CBCOA or an isomer, analog or derivative thereof.

In some embodiments, the crude lysate comprising the non-natural flavin-dependent oxidase is capable of converting CBGVA to CBCVA, CBDVA, THCVA, or an isomer, analog or derivative thereof. In some embodiments, the CBGVA is contacted with crude cell lysate comprising the non-natural flavin-dependent oxidase to form CBCVA, CBDVA, THCVA, or an isomer, analog or derivative thereof. In some embodiments, a partially purified lysate comprising the non-natural flavin-dependent oxidase is capable of converting CBGVA to CBCVA, CBDVA, THCVA, or an isomer, analog or derivative thereof. In some embodiments, the CBGVA is contacted with partially purified lysate comprising the non-natural flavin-dependent oxidase to form CBCVA, CBDVA, THCVA, or an isomer, analog or derivative thereof. In some embodiments, a purified non-natural flavin-dependent oxidase is capable of converting CBGVA to CBCVA, CBDVA, THCVA, or an isomer, analog or derivative thereof In some embodiments, the CBGVA is contacted with purified non-natural flavin-dependent oxidase to form CBCVA, CBDVA, THCVA, or an isomer, analog or derivative thereof. In some embodiments, the CBGVA is converted to CBCVA or an isomer, analog or derivative thereof.

In some embodiments, the crude lysate comprising the non-natural flavin-dependent oxidase is capable of converting CBG to CBC, CBD, THC, or an isomer, analog or derivative thereof. In some embodiments, the CBG is contacted with crude cell lysate comprising the non-natural flavin-dependent oxidase to form CBC, CBD, THC, or an isomer, analog or derivative thereof. In some embodiments, a partially purified lysate comprising the non-natural flavin-dependent oxidase is capable of converting CBG to CBC, CBD, THC, or an isomer, analog or derivative thereof. In some embodiments, the CBG is contacted with partially purified lysate comprising the non-natural flavin-dependent oxidase to form CBC, CBD, THC, or an isomer, analog or derivative thereof. In some embodiments, a purified non-natural flavin-dependent oxidase is capable of converting CBG to CBC, CBD, THC, or an isomer, analog or derivative thereof. In some embodiments, the CBG is contacted with purified non-natural flavin-dependent oxidase to form CBC, CBD, THC, or an isomer, analog or derivative thereof. In some embodiments, the CBG is converted to CBC or an isomer, analog or derivative thereof.

### Compositions

In some embodiments, the disclosure provides a composition comprising a cannabinoid or an isomer, analog or derivative thereof obtained from the engineered cell, cell extract, or method described herein. In some embodiments, the cannabinoid is CBCA, CBDA, THCA, CBCOA, CBDOA, THCOA, CBCVA, CBDVA, THCVA, CBC, CBD, THC, or an isomer, analog or derivative thereof, or a combination thereof. In some embodiments, the cannabinoid is CBCA, THCA, CBCOA, CBCVA, CBC, or an isomer, analog or derivative thereof, or a combination thereof.

In some embodiments, the cannabinoid is 10% or greater, 20% or greater, 30% or greater, 40% or greater, 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition. In some embodiments, the cannabinoid is CBCA or an isomer, analog or derivative thereof. In some embodiments, the cannabinoid is THCA or an isomer, analog or derivative thereof. In some embodiments, the cannabinoid is CBCOA or an isomer, analog or derivative thereof. In some embodiments, the cannabinoid is CBCVA or an isomer, analog or derivative thereof. In some embodiments, the cannabinoid is CBC or an isomer, analog or derivative thereof. In some embodiments, the cannabinoid comprises any combination of CBCA, CBDA, THCA, CBCOA, CBDOA, THCOA, CBCVA, CBDVA, THCVA, CBC, CBD, THC, or an isomer, analog, or derivative thereof. In some embodiments, the cannabinoid comprises any combination of CBCA, THCA, CBCOA, CBCVA, CBC, or an isomer, analog, or derivative thereof.

In some embodiments, the composition is a therapeutic or medicinal composition. In some embodiments, the composition further comprises a pharmaceutically acceptable excipient. In some embodiments, the composition is a topical composition. In some embodiments, the composition is in the form of a cream, a lotion, a paste, or an ointment.

In some embodiments, the composition is an edible composition. In some embodiments, the composition is provided in a food or beverage product. In some embodiments, the composition is an oral unit dosage composition. In some embodiments, the composition is provided in a tablet or a capsule.

In some embodiments, the disclosure provides a composition comprising (a) a non-natural flavin-dependent oxidase as described herein; and (b) a cannabinoid, a prenylated aromatic compound, or both. In some embodiments, the cannabinoid is CBCA, CBDA, THCA, CBCOA, CBDOA, THCOA, CBCVA, CBDVA, THCVA, CBC, CBD, THC, or an isomer, analog, or derivative thereof, or a combination thereof. In some embodiments, the cannabinoid or prenylated aromatic compound is CBGA, CBCA, THCA, CBGOA, CBCOA, CBGVA, CBCVA, CBG, CBC, or an isomer, analog, or derivative thereof, or a combination thereof.

In some embodiments, the disclosure provides a composition comprising (a) a flavin-dependent oxidase comprising any one of SEQ ID NOs:1-6; and (b) a cannabinoid, a prenylated aromatic compound, or both. In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:4. In some embodiments, the flavin-dependent oxidase comprises SEQ ID NO:5. In some embodiments, the flavin-dependent oxidase comprises SEQ ID NO:6. In some embodiments, the flavin-dependent oxidase is EncM. In some embodiments, the flavin-dependent oxidase is Clz9. In some embodiments, the cannabinoid or prenylated aromatic compound is CBGA, CBGOA, CBGVA, CBG, CBCA, CBDA, THCA, CBCOA, CBDOA, THCOA, CBCVA, CBDVA, THCVA, CBC, CBD, THC, or an isomer, analog, or derivative thereof, or a combination thereof. In some embodiments, the cannabinoid or prenylated aromatic compound is CBGA, CBCA, THCA, CBGOA, CBCOA, CBGVA, CBCVA, CBG, CBC, or an isomer, analog, or derivative thereof, or a combination thereof.

In some embodiments, the disclosure provides a composition comprising: (a) a flavin-dependent oxidase, wherein the flavin-dependent oxidase does not comprise a disulfide bond, and wherein the non-natural flavin-dependent oxidase is capable of oxidative cyclization of a prenylated aromatic compound into a cannabinoid; and (b) a cannabinoid, a prenylated aromatic compound, or both. In some embodiments, the cannabinoid or prenylated aromatic compound is CBGA, CBGOA, CBGVA, CBG, CBCA, CBDA, THCA, CBCOA, CBDOA, THCOA, CBCVA, CBDVA, THCVA, CBC, CBD, THC, or an isomer, analog, or derivative thereof, or a combination thereof. In some embodiments, the cannabinoid or prenylated aromatic compound is CBGA, CBCA, THCA, CBGOA, CBCOA, CBGVA, CBCVA, CBG, CBC, or an isomer, analog, or derivative thereof, or a combination thereof.

In some embodiments, the disclosure provides a composition comprising: (a) a flavin-dependent oxidase, wherein the flavin-dependent oxidase does not comprise a disulfide bond, and wherein the non-natural flavin-dependent oxidase is capable of oxidative cyclization of a prenylated aromatic compound into a cannabinoid; and (b) a cannabinoid, a prenylated aromatic compound, or both. Flavin-dependent oxidases that do not comprise disulfide bonds and capable of oxidative cyclization of a prenylated aromatic compound into a cannabinoid are described herein. In some embodiments, the cannabinoid or prenylated aromatic compound is CBGA, CBGOA, CBGVA, CBG, CBCA, CBDA, THCA, CBCOA, CBDOA, THCOA, CBCVA, CBDVA, THCVA, CBC, CBD, THC, or an isomer, analog, or derivative thereof, or a combination thereof. In some embodiments, the cannabinoid or prenylated aromatic compound is CBGA, CBCA, THCA, CBGOA, CBCOA, CBGVA, CBCVA, CBG, CBC, or an isomer, analog, or derivative thereof, or a combination thereof.

In some embodiments, the compositions herein comprising a flavin-dependent oxidase and a cannabinoid, a prenylated aromatic compound, or both, further comprise an enzyme in a cannabinoid biosynthesis pathway. Cannabinoid biosynthesis pathways are described herein. In some embodiments, the cannabinoid biosynthesis pathway enzyme comprises olivetol synthase (OLS), olivetolic acid cyclase (OAC), prenyltransferase, or a combination thereof.

All references cited herein, including patents, patent applications, papers, textbooks and the like, and the references cited therein, to the extent that they are not already, are hereby incorporated herein by reference in their entirety.

Sequences

SEQ ID NO: 1-Wild-type EncM
MQFPQLDPATLAAFSAAFRGELIWPSDADYDEARRIWNGTIDRRPALIARCTSTPDVVAAVSFARKSGLLVAVRGGGHSMAGHSVC
DGGIVIDLSLMNSIKVSRRLRRARAQGGCLLGAFDTATQAHMLATPAGVVSHTGLGGLVLGGGFGWLSRKYGLSIDNLTSVEIVTA
DGGVLTASDTENPDLFWAVRGGGGNFGVVTAFEFDLHRVGPVRFASTYYSLDEGPQVIRAWRDHMATAPDELTWALYLRLAPPLPE
LPADMHGKPVICAMSCWIGDPHEGERQLESILHAGKPHGLTKATLPYRALQAYSFPGAVVPDRIYTKSGYLNELSDEATDTVLEHA
ADIASPFTQLELLYLGGAVARVPDDATAYPNRQSPFVTNLAAAWMDPTEDARHTAWAREGYRALAGHLSGGYVNFMNPGEADRTRE
AYGAAKFERLQGVKAKYDPTNLFRLNQNIPPS
SEQ ID NO: 2-EncM T139V
MQFPQLDPATLAAFSAAFRGELIWPSDADYDEARRIWNGTIDRRPALIARCTSTPDVVAAVSFARKSGLLVAVRGGGHSMAGHSVC
DGGIVIDLSLMNSIKVSRRLRRARAQGGCLLGAFDTATQAHMLATPAGVVSHVGLGGLVLGGGFGWLSRKYGLSIDNLTSVEIVTA
DGGVLTASDTENPDLFWAVRGGGGNFGVVTAFEFDLHRVGPVRFASTYYSLDEGPQVIRAWRDHMATAPDELTWALYLRLAPPLPE
LPADMHGKPVICAMSCWIGDPHEGERQLESILHAGKPHGLTKATLPYRALQAYSFPGAVVPDRIYTKSGYLNELSDEATDTVLEHA
ADIASPFTQLELLYLGGAVARVPDDATAYPNRQSPFVTNLAAAWMDPTEDARHTAWAREGYRALAGHLSGGYVNFMNPGEADRTRE
AYGAAKFERLQGVKAKYDPTNLFRLNQNIPPS
SEQ ID NO: 3-Wild-type Clz9
MTADPSSERSDMNEADEVNEVDELSETGQTSGTKGKRPFTGRVIGPADGEFDEARRVWNECFAARPKEIVYCADTRDVVRALREVR
QRGGPFRVRSGGHSMSGLSVLDDGTVLDVSGLDDIQVSEDASTVTVGSGAHLGDIFRALWARGVTVPAGFCPEIGIAGHVLGGGAG
ILVRSRGFLSDHLVALEMVDSEGRIVVADHDSHHELLWASRGGGGGNFGIATSFTLRTQPIGDVTLFTIAWDWDRGAEAIKAWQEW
LATADGRINTLFIAYPQDQDMFAALGCFEGDAAELEPLIAPLVHAVEPTEKVAETMPWIEALSFVETMQGEAMKATSVRAKGNLSF
VTEPLGDRAVEEIKKALAQAPSHRAEVVLYGLGGAVAAKGRRETAFVHRDAPVALNYHTDWDDEAEDDLNFAWIQNLRASVAAHTE
GRGSYVNTIDLTVEHWLWDYYEENLPRLMAVKKRYDPEDVFRHPQSIPVSLTEAEAAELGIPPHIAEELRAARQLR
SEQ ID NO: 4-EncM with N-terminal 6xHis tag and thrombin cleavage site
MGSSHHHHHHSSGLVPRGSQFPQLDPATLAAFSAAFRGELIWPSDADYDEARRIWNGTIDRRPALIARCTSTPDVVAAVSFARKSG
LLVAVRGGGHSMAGHSVCDGGIVIDLSLMNSIKVSRRLRRARAQGGCLLGAFDTATQAHMLATPAGVVSHTGLGGLVLGGGFGWLS
RKYGLSIDNLTSVEIVTADGGVLTASDTENPDLFWAVRGGGGNFGVVTAFEFDLHRVGPVRFASTYYSLDEGPQVIRAWRDHMATA
PDELTWALYLRLAPPLPELPADMHGKPVICAMSCWIGDPHEGERQLESILHAGKPHGLTKATLPYRALQAYSFPGAVVPDRIYTKS
GYLNELSDEATDTVLEHAADIASPFTQLELLYLGGAVARVPDDATAYPNRQSPFVTNLAAAWMDPTEDARHTAWAREGYRALAGHL
SGGYVNFMNPGEADRTREAYGAAKFERLQGVKAKYDPTNLFRLNQNIPPS
SEQ ID NO: 5-EncM T139V with N-terminal 6xHis tag and thrombin cleavage site
MGSSHHHHHHSSGLVPRGSQFPQLDPATLAAFSAAFRGELIWPSDADYDEARRIWNGTIDRRPALIARCTSTPDVVAAVSFARKSG
LLVAVRGGGHSMAGHSVCDGGIVIDLSLMNSIKVSRRLRRARAQGGCLLGAFDTATQAHMLATPAGVVSHVGLGGLVLGGGFGWLS
RKYGLSIDNLTSVEIVTADGGVLTASDTENPDLFWAVRGGGGNFGVVTAFEFDLHRVGPVRFASTYYSLDEGPQVIRAWRDHMATA
PDELTWALYLRLAPPLPELPADMHGKPVICAMSCWIGDPHEGERQLESILHAGKPHGLTKATLPYRALQAYSFPGAVVPDRIYTKS
GYLNELSDEATDTVLEHAADIASPFTQLELLYLGGAVARVPDDATAYPNRQSPFVTNLAAAWMDPTEDARHTAWAREGYRALAGHL
SGGYVNFMNPGEADRTREAYGAAKFERLQGVKAKYDPTNLFRLNQNIPPS
SEQ ID NO: 6-Clz9 with N-terminal MBP tag
MGSSHHHHHHGSSGASEEGKLVIWINGDKGYNGLAEVGKKFEKDTGIKVTVEHPDKLEEKFPQVAATGDGPDIIFWAHDRFGGYAQ
SGLLAEITPDKAFQDKLYPFTWDAVRYNGKLIAYPIAVEALSLIYNKDLLPNPPKTWEEIPALDKELKAKGKSALMFNLQEPYFTW
PLIAADGGYAFKYENGKYDIKDVGVDNAGAKAGLTFLVDLIKNKHMNADTDYSIAEAAFNKGETAMTINGPWAWSNIDTSKVNYGV
TVLPTFKGQPSKPFVGVLSAGINAASPNKELAKEFLENYLLTDEGLEAVNKDKPLGAVALKSYEEELAKDPRIAATMENAQKGEIM
PNIPQMSAFWYAVRTAVINAASGRQTVDEALKDAQTNSITSLYGSSGSSGSNLYFQSNGMTADPSSERSDMNEADEVNEVDELSET
GQTSGTKGKRPFTGRVIGPADGEFDEARRVWNECFAARPKEIVYCADTRDVVRALREVRQRGGPFRVRSGGHSMSGLSVLDDGTVL
DVSGLDDIQVSEDASTVTVGSGAHLGDIFRALWARGVTVPAGFCPEIGIAGHVLGGGAGILVRSRGFLSDHLVALEMVDSEGRIVV
ADHDSHHELLWASRGGGGGNFGIATSFTLRTQPIGDVTLFTIAWDWDRGAEAIKAWQEWLATADGRINTLFIAYPQDQDMFAALGC
FEGDAAELEPLIAPLVHAVEPTEKVAETMPWIEALSFVETMQGEAMKATSVRAKGNLSFVTEPLGDRAVEEIKKALAQAPSHRAEV
VLYGLGGAVAAKGRRETAFVHRDAPVALNYHTDWDDEAEDDLNFAWIQNLRASVAAHTEGRGSYVNTIDLTVEHWLWDYYEENLPR
LMAVKKRYDPEDVFRHPQSIPVSLTEAEAAELGIPPHIAEELRAARQLR
SEQ ID NO: 7-OLS
MNHLRAEGPASVLAIGTANPENILLQDEFPDYYFRVTKSEHMTQLKEKFRKICDKSMIRKRNCFLNEEHLKQNPRLVEHEMQTLDA
RQDMLVVEVPKLGKDACAKAIKEWGQPKSKITHLIFTSASTTDMPGADYHCAKLLGLSPSVKRVMMYQLGCYGGGTVLRIAKDIAE
NNKGARVLAVCCDIMACLFRGPSESDLELLVGQAIFGDGAAAVIVGAEPDESVGERPIFELVSTGQTILPNSEGTIGGHIREAGLI
FDLHKDVPMLISNNIEKCLIEAFTPIGISDWNSIFWITHPGGKAILDKVEEKLHLKSDKFVDSRHVLSEHGNMSSSTVLFVMDELR
KRSLEEGKSTTGDGFEWGVLFGFGPGLTVERVVVRSVPIKY
SEQ ID NO: 8-OAC
MAVKHLIVLKFKDEITEAQKEEFFKTYVNLVNIIPAMKDVYWGKDVTQKNKEEGYTHIVEVTFESVETIQDYIIHPAHVGFGDVYR
SFWEKLLIFDYTPRK
SEQ ID NO: 9-Prenyltransferase
MSGAADVERVYAAMEEAAGLLGVTCAREKIYPLLTEFQDTLTDGVVVFSMASGRRSTELDFSISVPTSQGDPYATVVDKGLFPATG
HPVDDLLADTQKHLPVSMFAIDGEVTGGFKKTYAFFPTDDMPGVAQLSAIPSMPSSVAENAELFARYGLDKVQMTSMDYKKRQVNL
YFSELSEQTLAPESVLALVRELGLHVPTELGLEFCKRSFSVYPTLNWDTGKIDRLCFAVISTDPTLVPSTDERDIEQFRHYGTKAP
YAYVGENRTLVYGLTLSPTEEYYKLGAYYHITDIQRRLLKAFDALED
SEQ ID NO: 10-UniProt KB A0A2E0XWX6 from Phycisphaerae bacterium with C-
terminal linker and 6xHis tag
MQACNNHQLTDAILQEFSQTLSGDLVLPTDGLYQWARLIHHTNFDGTYPVGIVFCETPEDVSKAILFARQFGLHVTARGGGHSYEG
YSVTGGLLIDVSRMNSVSVNPAAMTAVVGAGAVLIDVYHGLYYPHKLSIPGGSCPSVGIAGYLLGGGVGLQSRTYGVGCDRVLEIG
VVLASGEYVVASPTNHSDLYWAYRGGGGGNFGVVTHFKMQCHPVDRLSYAIITWDWEAAAPAFNAWQNWLKGLGEDYRNFSIFKFL
VNAGGDGGLGPKVNLIVQFDSQSNTGTGELETLMAPLLNTAHEHIVSQTIMNGDFFEVTMEIMAGCAWPKTDLDTEFLRCHTVGNP
AFPMASLPRDTYKAKSTFFADVISEKGIETCIKAIEDRFNSNLPDNSSQFTCALQFDSEGGIMGDVPKDATAFMHRDCLMHCQYLA
YWPAEGDAWFDDNPDYSYCDVSNGSMQWISDAFNVLWPFGNGHAYQNYIDKEQPNWLYAYYGENVERLRTVKAKYDPDNIWKFEQS
IPPAQGGSGGSGSGSGGSGSHHHHHH
SEQ ID NO: 11-TamL from Streptomyces sp. 307-9 with N-terminal 6xHis tag and
thrombin cleavage site
MGSSHHHHHHSSGLVPRGSKHIDSVAPGDIRYEDLRRGENLRFVGDPEEIHLVGSAAEIEQVLSRAVRSGKRVAVRSGGHCYEDFV
ANSDVRVVMDMSRLSAVGFDEERGAFAVEAGATLGAVYKTLFRVWGVTLPGGACPDVGAGGHILGGGYGPLSRMHGSIVDYLHAVE
VVVVDASGDARTVIATREPSDPNHDLWWAHTGGGGGNFGVVVRYWLRTAEADVPPEPGRLLPRPPAEVLLNTTVWPWEGLDEAAFA
RLVRNHGRWFEQNSGPDSPWCDLYSVLALTRSQSGALAMTTQLDATGPDAEKRLETYLAAVSEGVGVQPHSDTRRLPWLHSTRWPG
IAGDGDMTGRAKIKAAYARRSFDDRQIGTLYTRLTSTDYDNPAGVVALIAYGGKVNAVPADRTAVAQRDSILKIVYVTTWEDPAQD
PVHVRWIRELYRDVYADTGGVPVPGGAADGAYVNYPDVDLADEEWNTSGVPWSELYYKDAYPRLQAVKARWDPRNVFRHALSVRVP
PA
SEQ ID NO: 12-Polynucleotide encoding SEQ ID NO: 4
ATGGGCAGCAGCCATCATCATCATCATCACAGCAGCGGCCTGGTGCCGCGCGGCAGCCAGTTCCCGCAGTTGGACCCTGCTACTTT
AGCCGCGTTCTCCGCAGCTTTCCGCGGCGAACTGATTTGGCCATCCGATGCAGACTACGATGAAGCGCGCCGTATCTGGAACGGTA
CTATCGACCGCCGTCCGGCTCTCATCGCGCGCTGTACTAGCACCCCGGACGTGGTTGCTGCGGTTTCCTTCGCGCGCAAATCTGGC
CTGCTTGTGGCGGTTCGTGGCGGTGGCCACTCTATGGCTGGTCACTCGGTATGCGACGGTGGCATTGTGATTGACCTGTCTCTGAT
GAACTCCATCAAAGTATCCCGCCGTCTGCGCCGTGCTCGTGCGCAGGGTGGCTGCCTGCTCGGTGCTTTCGACACTGCTACCCAGG
CGCATATGCTCGCGACCCCGGCTGGCGTCGTTTCCCACACCGGCCTGGGCGGTCTGGTTCTGGGTGGCGGTTTCGGCTGGCTGTCA
CGTAAATATGGCCTGAGCATCGACAACCTGACCTCTGTAGAAATCGTGACCGCTGACGGTGGCGTGCTGACCGCATCCGATACTGA
GAACCCGGACTTATTCTGGGCTGTTCGCGGAGGAGGAGGTAATTTTGGCGTCGTAACCGCTTTCGAATTCGACTTACACCGCGTCG
GCCCAGTTCGTTTCGCATCGACCTACTATAGCCTCGATGAAGGCCCGCAGGTCATCCGTGCTTGGCGTGACCACATGGCGACGGCA
CCGGATGAACTGACCTGGGCGCTCTATCTGCGTCTGGCGCCGCCACTGCCGGAACTGCCTGCAGACATGCACGGCAAACCGGTTAT
CTGCGCAATGTCTTGCTGGATTGGTGACCCACATGAAGGTGAACGTCAGTTAGAATCTATTCTGCATGCCGGTAAACCGCACGGCC
TGACTAAAGCGACCCTTCCGTACCGCGCACTGCAGGCCTATTCCTTCCCGGGCGCAGTCGTTCCGGACCGTATCTACACTAAATCC
GGGTACCTGAATGAGCTGTCCGACGAGGCGACCGATACCGTGCTTGAACACGCAGCGGATATCGCGTCGCCGTTCACTCAACTTGA
ACTGCTCTACCTGGGCGGTGCCGTTGCTCGTGTTCCAGATGACGCGACCGCTTATCCTAACCGCCAGTCTCCGTTTGTGACCAACC
TGGCGGCAGCTTGGATGGATCCGACCGAGGATGCTCGTCACACCGCTTGGGCGCGCGAAGGTTACCGTGCGCTGGCGGGCCATCTG
TCCGGTGGCTACGTTAACTTTATGAACCCGGGTGAGGCGGACCGTACCCGTGAAGCCTACGGCGCGGCAAAATTTGAGCGCCTGCA
GGGCGTGAAAGCTAAATATGACCCGACTAACCTGTTCCGTCTGAATCAGAACATTCCGCCATCCTAG
SEQ ID NO: 13-Polynucleotide encoding SEQ ID NO: 5
ATGGGCAGCAGCCATCATCATCATCATCACAGCAGCGGCCTGGTGCCGCGCGGCAGCCAGTTCCCGCAGTTGGACCCTGCTACTTT
AGCCGCGTTCTCCGCAGCTTTCCGCGGCGAACTGATTTGGCCATCCGATGCAGACTACGATGAAGCGCGCCGTATCTGGAACGGTA
CTATCGACCGCCGTCCGGCTCTCATCGCGCGCTGTACTAGCACCCCGGACGTGGTTGCTGCGGTTTCCTTCGCGCGCAAATCTGGC
CTGCTTGTGGCGGTTCGTGGCGGTGGCCACTCTATGGCTGGTCACTCGGTATGCGACGGTGGCATTGTGATTGACCTGTCTCTGAT
GAACTCCATCAAAGTATCCCGCCGTCTGCGCCGTGCTCGTGCGCAGGGTGGCTGCCTGCTCGGTGCTTTCGACACTGCTACCCAGG
CGCATATGCTCGCGACCCCGGCTGGCGTCGTTTCCCACGTGGGCCTGGGCGGTCTGGTTCTGGGTGGCGGTTTCGGCTGGCTGTCA
CGTAAATATGGCCTGAGCATCGACAACCTGACCTCTGTAGAAATCGTGACCGCTGACGGTGGCGTGCTGACCGCATCCGATACTGA
GAACCCGGACTTATTCTGGGCTGTTCGCGGAGGAGGAGGTAATTTTGGCGTCGTAACCGCTTTCGAATTCGACTTACACCGCGTCG
GCCCAGTTCGTTTCGCATCGACCTACTATAGCCTCGATGAAGGCCCGCAGGTCATCCGTGCTTGGCGTGACCACATGGCGACGGCA
CCGGATGAACTGACCTGGGCGCTCTATCTGCGTCTGGCGCCGCCACTGCCGGAACTGCCTGCAGACATGCACGGCAAACCGGTTAT
CTGCGCAATGTCTTGCTGGATTGGTGACCCACATGAAGGTGAACGTCAGTTAGAATCTATTCTGCATGCCGGTAAACCGCACGGCC
TGACTAAAGCGACCCTTCCGTACCGCGCACTGCAGGCCTATTCCTTCCCGGGCGCAGTCGTTCCGGACCGTATCTACACTAAATCC
GGGTACCTGAATGAGCTGTCCGACGAGGCGACCGATACCGTGCTTGAACACGCAGCGGATATCGCGTCGCCGTTCACTCAACTTGA
ACTGCTCTACCTGGGCGGTGCCGTTGCTCGTGTTCCAGATGACGCGACCGCTTATCCTAACCGCCAGTCTCCGTTTGTGACCAACC
TGGCGGCAGCTTGGATGGATCCGACCGAGGATGCTCGTCACACCGCTTGGGCGCGCGAAGGTTACCGTGCGCTGGCGGGCCATCTG
TCCGGTGGCTACGTTAACTTTATGAACCCGGGTGAGGCGGACCGTACCCGTGAAGCCTACGGCGCGGCAAAATTTGAGCGCCTGCA
GGGCGTGAAAGCTAAATATGACCCGACTAACCTGTTCCGTCTGAATCAGAACATTCCGCCATCCTAG
SEQ ID NO: 14-Polynucleotide encoding SEQ ID NO: 3
ATGACCGCAGATCCGAGCAGCGAACGTAGCGATATGAATGAAGCAGATGAAGTGAACGAAGTTGATGAACTGAGCGAAACCGGTCA
GACCAGCGGCACCAAAGGTAAACGTCCGTTTACAGGTCGTGTTATTGGTCCGGCAGATGGTGAATTTGATGAAGCACGTCGTGTTT
GGAATGAATGTTTTGCAGCACGTCCGAAAGAAATTGTTTATTGTGCAGATACCCGTGATGTTGTTCGTGCACTGCGTGAAGTTCGT
CAGCGTGGTGGTCCGTTTCGTGTTCGTAGCGGTGGTCATAGCATGAGCGGTCTGAGCGTTCTGGATGATGGTACAGTGCTGGATGT
TAGTGGCCTGGATGATATTCAGGTTAGCGAAGATGCAAGCACCGTTACCGTTGGTAGCGGTGCACATCTGGGTGATATTTTTCGTG
CCCTGTGGGCACGTGGTGTTACCGTTCCGGCAGGTTTTTGTCCGGAAATTGGTATTGCAGGTCATGTTTTAGGTGGTGGTGCAGGT
ATTCTGGTGCGTAGCCGTGGTTTTCTGAGCGATCATCTGGTTGCACTGGAAATGGTTGATAGCGAAGGTCGTATTGTTGTTGCAGA
TCATGATAGTCATCATGAACTGCTGTGGGCAAGCCGTGGTGGTGGCGGTGGTAATTTTGGCATTGCAACCAGCTTTACCCTGCGTA
CCCAGCCGATTGGTGATGTTACCCTGTTTACCATTGCATGGGATTGGGATCGTGGTGCCGAAGCAATTAAAGCATGGCAAGAATGG
CTGGCAACCGCAGATGGTCGCATTAATACACTGTTTATTGCATATCCGCAGGACCAGGATATGTTTGCAGCCCTGGGTTGTTTTGA
AGGTGATGCAGCAGAACTGGAACCGCTGATTGCACCGCTGGTTCATGCAGTTGAACCGACCGAAAAAGTTGCAGAAACCATGCCGT
GGATTGAAGCACTGAGCTTTGTTGAAACAATGCAGGGTGAAGCCATGAAAGCAACCAGCGTTCGTGCAAAAGGTAATCTGAGTTTT
GTTACCGAACCGCTGGGTGATCGTGCCGTTGAAGAAATCAAAAAAGCACTGGCACAGGCACCGAGCCATCGTGCCGAAGTTGTTCT
GTATGGTTTAGGTGGCGCAGTTGCAGCAAAAGGTCGTCGTGAAACCGCATTTGTTCATCGTGATGCACCGGTTGCGCTGAATTATC
ATACCGATTGGGATGATGAAGCCGAAGATGATCTGAATTTTGCCTGGATTCAGAATCTGCGTGCAAGCGTTGCAGCACATACCGAA
GGTCGCGGTAGCTATGTTAATACCATTGATCTGACCGTTGAACATTGGCTGTGGGATTATTATGAAGAAAATCTGCCTCGTCTGAT
GGCCGTGAAAAAACGTTATGATCCGGAAGATGTTTTTCGTCATCCGCAGAGCATTCCGGTTAGCCTGACCGAAGCAGAAGCAGCCG
AACTGGGTATTCCGCCTCATATTGCCGAAGAACTGCGTGCCGCACGTCAGCTGCGTTAG
SEQ ID NO: 15-Polynucleotide encoding SEQ ID NO: 6
ATGGGTAGCAGCCATCACCATCATCATCATGGTAGCAGCGGTGCAAGCGAAGAAGGCAAACTGGTTATTTGGATTAATGGCGATAA
AGGCTATAATGGTCTGGCAGAAGTTGGCAAAAAATTCGAAAAAGATACCGGCATTAAAGTGACCGTTGAACATCCGGATAAACTGG
AAGAAAAATTTCCGCAGGTTGCAGCAACCGGTGATGGTCCGGATATTATCTTTTGGGCACATGATCGTTTTGGTGGTTATGCACAG
AGCGGTCTGCTGGCAGAAATTACACCGGATAAAGCATTTCAGGACAAACTGTATCCGTTTACCTGGGATGCAGTTCGCTATAACGG
TAAACTGATTGCATATCCGATTGCAGTTGAAGCACTGAGCCTGATCTATAACAAAGATCTGCTGCCGAATCCGCCTAAAACCTGGG
AAGAAATTCCGGCACTGGATAAAGAACTGAAAGCAAAAGGTAAAAGCGCACTGATGTTTAATCTGCAAGAACCGTATTTTACCTGG
CCTCTGATTGCAGCAGATGGTGGCTATGCATTCAAATATGAAAACGGCAAATACGATATCAAGGATGTTGGTGTTGATAATGCGGG
TGCAAAAGCCGGTCTGACCTTTCTGGTTGATCTGATCAAAAACAAACACATGAATGCCGATACCGATTATAGCATTGCAGAAGCAG
CATTTAACAAAGGTGAAACCGCAATGACAATTAATGGTCCGTGGGCATGGTCAAATATTGATACCAGCAAAGTGAATTATGGTGTT
ACCGTTCTGCCGACATTTAAAGGTCAGCCGAGCAAACCGTTTGTTGGTGTGCTGAGCGCAGGTATTAATGCAGCAAGCCCGAACAA
AGAACTGGCAAAAGAATTTCTGGAAAACTATCTGCTGACCGATGAAGGTCTGGAAGCAGTGAATAAAGATAAACCGCTGGGTGCAG
TTGCACTGAAAAGCTATGAAGAGGAATTAGCAAAAGATCCGCGTATTGCAGCCACCATGGAAAATGCACAGAAAGGCGAAATTATG
CCGAATATTCCGCAGATGAGCGCATTTTGGTATGCCGTTCGTACCGCAGTGATTAATGCCGCATCAGGTCGTCAGACCGTTGATGA
AGCCCTGAAAGATGCCCAGACCAATAGCATTACCAGCCTGTATGGTAGCAGCGGTAGCTCAGGTAGCAATCTGTATTTTCAGAGCA
ATGGCATGACCGCAGATCCGAGCAGCGAACGTAGCGATATGAATGAAGCAGATGAAGTGAACGAAGTTGATGAACTGAGCGAAACC
GGTCAGACCAGCGGCACCAAAGGTAAACGTCCGTTTACAGGTCGTGTTATTGGTCCGGCAGATGGTGAATTTGATGAAGCACGTCG
TGTTTGGAATGAATGTTTTGCAGCACGTCCGAAAGAAATTGTTTATTGTGCAGATACCCGTGATGTTGTTCGTGCACTGCGTGAAG
TTCGTCAGCGTGGTGGTCCGTTTCGTGTTCGTAGCGGTGGTCATAGCATGAGCGGTCTGAGCGTTCTGGATGATGGTACAGTGCTG
GATGTTAGTGGCCTGGATGATATTCAGGTTAGCGAAGATGCAAGCACCGTTACCGTTGGTAGCGGTGCACATCTGGGTGATATTTT
TCGTGCCCTGTGGGCACGTGGTGTTACCGTTCCGGCAGGTTTTTGTCCGGAAATTGGTATTGCAGGTCATGTTTTAGGTGGTGGTG
CAGGTATTCTGGTGCGTAGCCGTGGTTTTCTGAGCGATCATCTGGTTGCACTGGAAATGGTTGATAGCGAAGGTCGTATTGTTGTT
GCAGATCATGATAGTCATCATGAACTGCTGTGGGCAAGCCGTGGTGGTGGCGGTGGTAATTTTGGCATTGCAACCAGCTTTACCCT
GCGTACCCAGCCGATTGGTGATGTTACCCTGTTTACCATTGCATGGGATTGGGATCGTGGTGCCGAAGCAATTAAAGCATGGCAAG
AATGGCTGGCAACCGCAGATGGTCGCATTAATACACTGTTTATTGCATATCCGCAGGACCAGGATATGTTTGCAGCCCTGGGTTGT
TTTGAAGGTGATGCAGCAGAACTGGAACCGCTGATTGCACCGCTGGTTCATGCAGTTGAACCGACCGAAAAAGTTGCAGAAACCAT
GCCGTGGATTGAAGCACTGAGCTTTGTTGAAACAATGCAGGGTGAAGCCATGAAAGCAACCAGCGTTCGTGCAAAAGGTAATCTGA
GTTTTGTTACCGAACCGCTGGGTGATCGTGCCGTTGAAGAAATCAAAAAAGCACTGGCACAGGCACCGAGCCATCGTGCCGAAGTT
GTTCTGTATGGTTTAGGTGGCGCAGTTGCAGCAAAAGGTCGTCGTGAAACCGCATTTGTTCATCGTGATGCACCGGTTGCGCTGAA
TTATCATACCGATTGGGATGATGAAGCCGAAGATGATCTGAATTTTGCCTGGATTCAGAATCTGCGTGCAAGCGTTGCAGCACATA
CCGAAGGTCGCGGTAGCTATGTTAATACCATTGATCTGACCGTTGAACATTGGCTGTGGGATTATTATGAAGAAAATCTGCCTCGT
CTGATGGCCGTGAAAAAACGTTATGATCCGGAAGATGTTTTTCGTCATCCGCAGAGCATTCCGGTTAGCCTGACCGAAGCAGAAGC
AGCCGAACTGGGTATTCCGCCTCATATTGCCGAAGAACTGCGTGCCGCACGTCAGCTGCGTTAG
SEQ ID NO: 16-Polynucleotide encoding SEQ ID NO: 10
ATGCAGGCATGTAATAATCATCAGCTGACCGATGCAATCCTGCAAGAATTTTCACAGACCCTGAGCGGTGATCTGGTTCTGCCGAC
CGATGGTCTGTATCAGTGGGCACGTCTGATTCATCATACCAATTTTGATGGTATTTATCCGGTGGGCATTGTGTTTTGTGAAACAC
CGGAAGATGTTAGCAAAGCAATTCTGTTTGCACGTCAGTTTGGTCTGCATGTTACCGCACGTGGTGGTGGTCATAGTTATGAAGGT
TATAGCGTTACCGGTGGTCTGCTGATTGATGTTAGCCGTATGAATAGCGTTAGCGTTAATCCGGCAGCAATGACCGCAGTTGTTGG
TGCCGGTGCAGTTCTGATCGATGTTTATCATGGCCTGTATTATCCGCACAAACTGAGCATTCCAGGTGGTAGCTGTCCGAGCGTTG
GTATTGCAGGTTATCTGCTTGGTGGTGGCGTGGGTCTGCAGAGCCGTACCTATGGTGTTGGTTGTGATCGTGTTCTGGAAATTGGT
GTTGTTCTGGCAAGCGGTGAATATGTTGTTGCAAGCCCGACCAATCATAGCGATCTGTATTGGGCATATCGTGGTGGCGGTGGTGG
TAATTTTGGTGTGGTTACCCACTTTAAAATGCAGTGTCATCCGGTTGATCGTCTGAGCTATGCAATTATTACCTGGGATTGGGAAG
CAGCAGCACCGGCATTTAATGCATGGCAGAATTGGCTGAAAGGTCTGGGTGAAGATTATCGCAATTTCTCGATCTTTAAGTTCCTG
GTTAATGCAGGCGGTGATGGTGGTCTGGGTCCGAAAGTTAATCTGATTGTTCAGTTTGATAGCCAGAGCAATACCGGCACCGGTGA
ACTGGAAACCCTGATGGCACCGCTGCTGAATACCGCACATGAACATATTGTTAGCCAGACCATTATGAACGGCGATTTTTTTGAAG
TGACCATGGAAATTATGGCAGGTTGTGCATGGCCGAAAACCGATCTGGATACCGAATTTCTGCGTTGTCATACCGTTGGCAATCCG
GCATTTCCGATGGCAAGCCTGCCTCGTGATACCTATAAAGCAAAAAGCACCTTTTTTGCCGATGTGATTAGCGAAAAAGGTATCGA
AACCTGCATCAAAGCCATTGAAGATCGCTTTAATAGCAATCTGCCGGATAATAGCAGCCAGTTTACCTGTGCACTGCAGTTCGATA
GCGAAGGTGGTATTATGGGTGATGTTCCGAAAGATGCAACCGCATTTATGCATCGTGATTGTCTGATGCATTGTCAGTATCTGGCA
TATTGGCCTGCCGAAGGTGATGCATGGTTTGATGATAACCCGGATTATAGCTATTGTGATGTGAGCAATGGTAGCATGCAGTGGAT
TAGTGATGCCTTTAATGTTCTGTGGCCGTTTGGTAATGGTCATGCATATCAGAACTATATCGATAAAGAACAGCCGAACTGGCTGT
ATGCATATTATGGTGAAAATGTTGAACGTCTGCGTACCGTGAAAGCAAAATATGATCCGGATAACATCTGGAAGTTTGAACAGAGC
ATTCCGCCTGCATAGGGCGGTAGCGGAGGATCTGGTAGTGGCTCTGGAGGATCTGGTAGTCACCATCATCATCACCAT
SEQ ID NO: 17-Polynucleotide encoding SEQ ID NO: 11
ATGGGCAGCAGCCATCATCATCATCATCACAGCAGCGGCCTGGTGCCGCGCGGCAGCAAACACATTGACTCGGTTGCGCCGGGAGA
CATCCGTTACGAGGATTTGCGCCGCGGTGAAAACTTACGTTTCGTTGGTGATCCAGAGGAAATTCACTTGGTCGGTTCGGCAGCGG
AAATTGAACAGGTTCTTAGTCGCGCGGTGCGCAGTGGGAAACGTGTCGCGGTACGCTCTGGCGGGCATTGCTACGAAGATTTTGTT
GCGAACAGCGACGTGCGCGTGGTTATGGATATGTCCCGCTTAAGCGCAGTGGGCTTTGACGAGGAGCGCGGCGCTTTTGCCGTAGA
GGCTGGGGCCACGCTGGGCGCAGTATACAAGACCTTGTTTCGCGTATGGGGAGTGACTCTGCCAGGTGGCGCCTGTCCTGATGTAG
GCGCTGGCGGACATATCCTTGGCGGTGGATATGGTCCTTTGTCACGCATGCATGGGTCGATCGTCGATTATCTTCACGCTGTTGAG
GTGGTCGTCGTCGACGCTTCTGGAGACGCCCGTACTGTAATCGCTACCCGCGAGCCGAGCGACCCTAACCACGATTTATGGTGGGC
GCACACTGGAGGTGGTGGTGGGAACTTCGGGGTAGTCGTACGCTACTGGCTTCGTACAGCGGAGGCCGACGTACCTCCGGAGCCTG
GGCGCCTGTTGCCCCGTCCACCAGCTGAAGTCTTGCTGAATACTACAGTGTGGCCCTGGGAGGGATTAGACGAGGCCGCGTTTGCT
CGTCTGGTGCGTAATCACGGCCGTTGGTTCGAACAAAACTCGGGACCTGATTCGCCTTGGTGTGACCTTTATAGTGTCTTAGCGTT
GACCCGCTCACAGTCCGGTGCGTTAGCTATGACAACTCAGCTTGACGCAACGGGACCTGACGCCGAAAAACGTCTTGAGACATATC
TTGCGGCTGTTAGCGAAGGCGTTGGGGTTCAGCCCCATTCCGATACACGCCGCTTGCCCTGGTTGCATTCGACACGCTGGCCAGGC
ATCGCCGGCGATGGCGACATGACGGGACGTGCGAAGATTAAGGCTGCCTATGCCCGTCGCAGTTTTGACGATCGCCAAATTGGCAC
ATTATACACACGTCTGACGAGCACCGATTATGATAACCCTGCTGGAGTCGTGGCTCTGATTGCTTATGGCGGAAAAGTCAACGCAG
TACCTGCCGACCGTACTGCCGTAGCGCAGCGCGATTCCATTCTGAAGATTGTATATGTTACGACTTGGGAAGACCCCGCTCAAGAT
CCTGTGCATGTGCGTTGGATCCGCGAGTTATACCGTGACGTTTACGCCGACACGGGGGGTGTGCCTGTTCCTGGGGGTGCGGCCGA
TGGAGCTTACGTTAACTACCCCGACGTGGATTTGGCGGACGAGGAATGGAATACTTCGGGGGTCCCGTGGAGCGAGTTATATTACA
AGGATGCCTATCCTCGTCTGCAAGCTGTGAAAGCACGCTGGGACCCGCGCAACGTTTTCCGTCACGCTTTGTCTGTCCGTGTCCCG
CCAGCTTAG
SEQ ID NO: 18-Clz9 with N-terminal histidine tag and thrombin cleavage site
MKHHHHHHHHGGLVPRGSHGTADPSSERSDMNEADEVNEVDELSETGQTSGTKGKRPFTGRVIGPADGEFDEARRVWNECFAARPK
EIVYCADTRDVVRALREVRQRGGPFRVRSGGHSMSGLSVLDDGTVLDVSGLDDIQVSEDASTVTVGSGAHLGDIFRALWARGVTVP
AGFCPEIGIAGHVLGGGAGILVRSRGFLSDHLVALEMVDSEGRIVVADHDSHHELLWASRGGGGGNFGIATSFTLRTQPIGDVTLF
TIAWDWDRGAEAIKAWQEWLATADGRINTLFIAYPQDQDMFAALGCFEGDAAELEPLIAPLVHAVEPTEKVAETMPWIEALSFVET
MQGEAMKATSVRAKGNLSFVTEPLGDRAVEEIKKALAQAPSHRAEVVLYGLGGAVAAKGRRETAFVHRDAPVALNYHTDWDDEAED
DLNFAWIQNLRASVAAHTEGRGSYVNTIDLTVEHWLWDYYEENLPRLMAVKKRYDPEDVFRHPQSIPVSLTEAEAAELGIPPHIAE
ELRAARQLR
SEQ ID NO: 19-Clz9 with 14-amino acid N-terminal truncation
ADEVNEVDELSETGQTSGTKGKRPFTGRVIGPADGEFDEARRVWNECFAARPKEIVYCADTRDVVRALREVRQRGGPFRVRSGGHS
MSGLSVLDDGTVLDVSGLDDIQVSEDASTVTVGSGAHLGDIFRALWARGVTVPAGFCPEIGIAGHVLGGGAGILVRSRGFLSDHLV
ALEMVDSEGRIVVADHDSHHELLWASRGGGGGNFGIATSFTLRTQPIGDVTLFTIAWDWDRGAEAIKAWQEWLATADGRINTLFIA
YPQDQDMFAALGCFEGDAAELEPLIAPLVHAVEPTEKVAETMPWIEALSFVETMQGEAMKATSVRAKGNLSFVTEPLGDRAVEEIK
KALAQAPSHRAEVVLYGLGGAVAAKGRRETAFVHRDAPVALNYHTDWDDEAEDDLNFAWIQNLRASVAAHTEGRGSYVNTIDLTVE
HWLWDYYEENLPRLMAVKKRYDPEDVFRHPQSIPVSLTEAEAAELGIPPHIAEELRAARQLR
SEQ ID NO: 20-Clz9 with 29-amino acid N-terminal truncation
TSGTKGKRPFTGRVIGPADGEFDEARRVWNECFAARPKEIVYCADTRDVVRALREVRQRGGPFRVRSGGHSMSGLSVLDDGTVLDV
SGLDDIQVSEDASTVTVGSGAHLGDIFRALWARGVTVPAGFCPEIGIAGHVLGGGAGILVRSRGFLSDHLVALEMVDSEGRIVVAD
HDSHHELLWASRGGGGGNFGIATSFTLRTQPIGDVTLFTIAWDWDRGAEAIKAWQEWLATADGRINTLFIAYPQDQDMFAALGCFE
GDAAELEPLIAPLVHAVEPTEKVAETMPWIEALSFVETMQGEAMKATSVRAKGNLSFVTEPLGDRAVEEIKKALAQAPSHRAEVVL
YGLGGAVAAKGRRETAFVHRDAPVALNYHTDWDDEAEDDLNFAWIQNLRASVAAHTEGRGSYVNTIDLTVEHWLWDYYEENLPRLM
AVKKRYDPEDVFRHPQSIPVSLTEAEAAELGIPPHIAEELRAARQLR

### Exemplary Embodiments

Embodiment 1 includes a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid, wherein: (i) the non-natural flavin-dependent oxidase comprises at least 70% sequence identity to SEQ ID NO:1, e.g., at least 80% sequence identity, at least 85% sequence identity, or at least 90% sequence identity to SEQ ID NO:1, and wherein the at least one amino acid variation comprises a substitution at position V136, S137, T139, L144, Y249, F313, Q353, or a combination thereof, wherein the amino acid position corresponds to SEQ ID NO:1; or (ii) wherein the non-natural flavin-dependent oxidase comprises at least 70% sequence identity to SEQ ID NO:3, e.g., at least 80% sequence identity, at least 85% sequence identity, or at least 90% sequence identity to SEQ ID NO:3, and wherein the at least one amino acid variation comprises a substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or (iii) the non-natural flavin-dependent oxidase comprises at least 70% sequence identity to SEQ ID NO:3, e.g., at least 80% sequence identity, at least 85% sequence identity, or at least 90% sequence identity to SEQ ID NO: 3, and wherein the at least one amino acid variation comprises a deletion of about 5 to about 50 amino acid residues at an N-terminus of SEQ ID NO:3, optionally comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or (iv) wherein the non-natural flavin-dependent oxidase comprises at least 70% sequence identity to SEQ ID NO:19 or 20, e.g., at least 80% sequence identity, at least 85% sequence identity, or at least 90% sequence identity to SEQ ID NO:19 or 20, optionally comprising an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

Embodiment 2 includes the non-natural flavin-dependent oxidase of embodiment 1, wherein the non-natural flavin-dependent oxidase is a berberine bridge enzyme (BBE)-like enzyme.

Embodiment 3 includes the non-natural flavin-dependent oxidase of embodiment 1 or 2, wherein the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG).

Embodiment 4 includes the non-natural flavin-dependent oxidase of any one of embodiments 1 to 3, wherein the non-natural flavin-dependent oxidase has at least 75% sequence identity to SEQ ID NO:1, 3, 19, or 20.

Embodiment 5 includes the non-natural flavin-dependent oxidase of any one of embodiments 1 to 4, wherein the non-natural flavin-dependent oxidase has at least 80% sequence identity to SEQ ID NO:1, 3, 19, or 20.

Embodiment 6 includes the non-natural flavin-dependent oxidase of any one of embodiments 1 to 5, wherein the non-natural flavin-dependent oxidase is not glycosylated.

Embodiment 7 includes the non-natural flavin-dependent oxidase of any one of embodiments 1 to 6, wherein the non-natural flavin-dependent oxidase comprises a monovalently bound FAD cofactor.

Embodiment 8 includes the non-natural flavin-dependent oxidase of any one of embodiments 1 to 6, wherein the non-natural flavin-dependent oxidase comprises a bivalently bound FAD cofactor.

Embodiment 9 includes the non-natural flavin-dependent oxidase of any one of embodiments 1 to 8, wherein the non-natural flavin-dependent oxidase is capable of oxidative cyclization of a prenylated aromatic compound into a cannabinoid at about pH 7.5.

Embodiment 10 includes the non-natural flavin-dependent oxidase of any one of embodiments 1 to 9, wherein catalytic activity of the non-natural flavin-dependent oxidase is substantially the same from about pH 5 to about pH 8.

Embodiment 11 includes the non-natural flavin-dependent oxidase of any one of embodiments 1 to 10, wherein the at least one amino acid variation comprises a substitution, deletion, insertion, or a combination thereof.

Embodiment 12 includes the non-natural flavin-dependent oxidase of any one of embodiments 1 to 11, having at least 90% sequence identity to SEQ ID NO:1.

Embodiment 13 includes the non-natural flavin-dependent oxidase of any one of embodiments 1 to 12, having at least 95% sequence identity to SEQ ID NO:1.

Embodiment 14 includes the non-natural flavin-dependent oxidase of embodiment 12 or 13, wherein the variation comprises an amino acid substitution selected from V136C, S137P, T139V, L144H, Y249H, F313A, Q353N, or a combination thereof, wherein the amino acid position corresponds to SEQ ID NO:1.

Embodiment 15 includes the non-natural flavin-dependent oxidase of embodiment 14, wherein the variation comprises a T139V substitution.

Embodiment 16 includes the non-natural flavin-dependent oxidase of any one of embodiments 12 to 15, wherein the non-natural flavin-dependent oxidase converts CBGA to cannabichromenic acid (CBCA), tetrahydrocannabinolic acid (THCA), or both.

Embodiment 17 includes the non-natural flavin-dependent oxidase of embodiment 16, wherein the non-natural flavin-dependent oxidase converts CBGA to CBCA at about pH 4 to about pH 9.

Embodiment 18 includes the non-natural flavin-dependent oxidase of any one of embodiments 12 to 17, wherein the non-natural flavin-dependent oxidase converts CBGOA to cannabiorcichromenic acid (CBCOA).

Embodiment 19 includes the non-natural flavin-dependent oxidase of any one of embodiments 12 to 18, wherein the non-natural flavin-dependent oxidase converts CBGVA to cannabichromevarinic acid (CBCVA).

Embodiment 20 includes the non-natural flavin-dependent oxidase of any one of embodiments 12 to 19, wherein the non-natural flavin-dependent oxidase converts CBG to cannabichromene (CBC).

Embodiment 21 includes the non-natural flavin-dependent oxidase of embodiment any one of embodiments 1 to 11, having at least 90% sequence identity to SEQ ID NO:3.

Embodiment 22 includes the non-natural flavin-dependent oxidase of any one of embodiments 1 to 11 or 21, having at least 95% sequence identity to SEQ ID NO:3.

Embodiment 23 includes the non-natural flavin-dependent oxidase of embodiment any one of embodiments 1 to 11, having at least 90% sequence identity to SEQ ID NO:19 or 20.

Embodiment 24 includes the non-natural flavin-dependent oxidase of any one of embodiments 1 to 11 or 21, having at least 95% sequence identity to SEQ ID NO:19 or 20.

Embodiment 25 includes the non-natural flavin-dependent oxidase of any one of embodiment 21 to 24, wherein the variation comprises an amino acid substitution selected from W58Q, W58H, W58K, W58G, W58V, M101A, M101S, M101F, M101Y, L104M, L104H, I160V, G161C, G161A, G161Q, G161L, A163G, V167F, L168S, L168G, A171Y, A171F, N267V, N267M, N267L, L269M, L269T, L269A, L269R, I271H, I271R, Y2731, Y273R, Q275K, Q275R, A281R, L283V, C285L, E287H, E287L, V323F, V323Y, V336F, A338I, G340L, L342Y, E370M, E370Q, V372A, V372E, V372I, V372L, V372T, V372C, A398E, A398V, N400W, H402T, H402I, H402V, H402A, H402M, H402Q, D404S, D404T, D404A, V436L, T438A, T438Y, T438F, or a combination thereof, wherein the amino acid position corresponds to SEQ ID NO:3.

Embodiment 26 includes the non-natural flavin-dependent oxidase of embodiment 25, wherein the variation comprises an amino acid substitution selected from T438A, T438Y, N400W, D404A, or a combination thereof, wherein the amino acid position corresponds to SEQ ID NO:3.

Embodiment 27 includes the non-natural flavin-dependent oxidase of embodiment 25, wherein the variation comprises an amino acid substitution at position D404 and an amino acid substitution at position L269, Y273, Q275, L283, C285, V323, E370, V372, N400, H402, T438, or a combination thereof, wherein the amino acid position corresponds to SEQ ID NO:3.

Embodiment 28 includes the non-natural flavin-dependent oxidase of embodiment 27, wherein the variation comprises D404A and one of: L269R, L269T, Q275R, Y273R, L283V, C285L, V323F, V323Y, E370M, E370Q, V372I, N400W, H402A, H402I, H402M, H402T, H402V, T438A, T438F, or T438Y.

Embodiment 29 includes the non-natural flavin-dependent oxidase of embodiment 25, wherein the variation comprises an amino acid substitution at position D404, an amino acid substitution at position T438, and an amino acid substitution at position L269, Y273, Q275, L283, C285, V323, E370, V372, N400, H402, T438, or a combination thereof, wherein the amino acid position corresponds to SEQ ID NO:3.

Embodiment 30 includes the non-natural flavin-dependent oxidase of embodiment 29, wherein the variation comprises: (a) D404A, T438F, and N400W; (b) D404A, T438F, and V323F; (c) D404A, T438F, and V323Y; (d) D404A, T438F, and E370M; (e) D404A, T438F, and H402I; (0 D404A, T438F, and E370Q; (g) D404A, T438F, and C285L; (h) T438F, N400W, and D404S; (i) T438F, V323Y, and D404S; (j) T438F, H402I, and D404S; (k) T438F, E370Q, and D404S; (l) D404A, T438F, V372I, and N400W; (m) D404A, T438F, V323Y, and N400W; (n) D404A, T438F, E370Q, and N400W; (o) D404A, T438F, V323Y, and E370M; (p) D404A, T438F, E370M, and N400W; (q) D404A, T438F, V323F, and H402I; (r) D404A, T438F, C285L, and N400W; (s) D404A, T438F, V323F, and N400W; (t) D404A, T438F, E370Q, and H402T; (u) D404A, T438F, N400W, and H402T; (v) D404A, T438F, V323F, and H402T; (w) D404A, T438F, C285L, and V323F; (x) D404A, T438F, L283V, and N400W; (y) D404A, T438F, V323F, and E370M; (z) D404A, T438F, Q275R, and N400W; (aa) D404A, T438F, V323Y, and H402T; (bb) D404A, T438F, V323F, and V372I; (cc) D404A, T438F, C285L, and V323Y; (dd) D404A, T438F, E370Q, and H402I; (ee) D404A, T438F, V323Y, and E370Q; (ff) D404A, T438F, Y273R, and V323Y; (gg) D404A, T438F, Y273R, and N400W; (hh) D404A, T438F, Y273R, and V323F; (ii) D404A, T438F, E370M, and H402T; (jj) D404A, T438F, L269T, and N400W; (kk) D404A, T438F, Q275R, and V323Y; (ll) D404A, T438F, V323Y, and H402I; (mm) D404A, T438F, V323F, and E370Q; (nn) D404A, T438F, Y273R, and Q275R; (oo) D404A, T438F, C285L, and E370Q; (pp) D404A, T438F, L283V, and V323Y; (qq) D404A, T438F, Y273R, and H402I; (rr) D404A, T438F, L269T, and E370M; (ss) D404A, T438F, C285L, and H402T; (tt) D404A, T438F, L269R, and N400W; (uu) D404A, T438F, Y273R, and C285L; (vv) D404A, T438F, L283V, and H402I; (ww) D404A, T438F, Q275R, and E370Q; (xx) D404A, T438F, V372I, and H402I; (yy) D404A, T438F, L283V, and E370Q; or (zz) D404A, T438F, V372I, and H402T.

Embodiment 31 includes the non-natural flavin-dependent oxidase of embodiment 30, wherein the variation comprises D404A, N400W, and V323Y.

Embodiment 32 includes the non-natural flavin-dependent oxidase of embodiment 30, wherein the variation comprises D404A, T438F, N400W, and V323Y.

Embodiment 33 includes the non-natural flavin-dependent oxidase of embodiment 25, wherein the variation comprises an amino acid substitution at position D404, an amino acid substitution at position T438, an amino acid substitution at position N400, an amino acid substitution at position V323, and an amino acid substitution at position L269, I271, Q275, A281, L283, C285, E370, V372, H402, or a combination thereof.

Embodiment 34 includes the non-natural flavin-dependent oxidase of embodiment 30, wherein the variation comprises D404A, T438F, N400W, V323Y, and one or more of: L269M, I271H, Q275R, A281R, L283S, C285L, E370M, E370Q, V372I, and H402T.

Embodiment 35 includes the non-natural flavin-dependent oxidase of embodiment 34, wherein the variation comprises: (a) D404A, T438F, N400W, V323Y, and E370Q; (b) D404A, T438F, N400W, V323Y, and V372I; (c) D404A, T438F, N400W, V323Y, and L269M; (d) D404A, T438F, N400W, V323Y, and C285L; (e) D404A, T438F, N400W, V323Y, and A281R; (0 D404A, T438F, N400W, V323Y, I271H, and E370Q; (g) D404A, T438F, N400W, V323Y, E370Q, and V372I; (h) D404A, T438F, N400W, V323Y, L269M, and E370Q; (i) D404A, T438F, N400W, V323Y, C285L, and E370Q; (j) D404A, T438F, N400W, V323Y, Q275R, and E370Q; (k) D404A, T438F, N400W, V323Y, L283S, and E370Q; (l) D404A, T438F, N400W, V323Y, A281R, and C285L; (m) D404A, T438F, N400W, V323Y, Q275R, and V372I; (n) D404A, T438F, N400W, V323Y, C285L, and E370M; (o) D404A, T438F, N400W, V323Y, L269M, and V372I; (p) D404A, T438F, N400W, V323Y, Q275R, and C285L; (q) D404A, T438F, N400W, V323Y, I271H, and L283S; (r) D404A, T438F, N400W, V323Y, Q275R, and A281R; (s) D404A, T438F, N400W, V323Y, L269M, and I271H; (t) D404A, T438F, N400W, V323Y, I271H, and E370M; (u) D404A, T438F, N400W, V323Y, I271H, and C285L; (v) D404A, T438F, N400W, V323Y, A281R, and V372I; (w) D404A, T438F, N400W, V323Y, E370M, and V372I; (x) D404A, T438F, N400W, V323Y, L269M, and Q275R; (y) D404A, T438F, N400W, V323Y, C285L, and V372I; (z) D404A, T438F, N400W, V323Y, V372I, and H402T; (aa) D404A, T438F, N400W, V323Y, L269M, and E370M; (bb) D404A, T438F, N400W, V323Y, Q275R, and E370M; (cc) D404A, T438F, N400W, V323Y, A281R, and E370Q; or (dd) D404A, T438F, N400W, V323Y, A281R, and L283S.

Embodiment 36 includes the non-natural flavin-dependent oxidase of any one of embodiments 21 to 35, wherein the non-natural flavin-dependent oxidase does not comprise a variation at any of amino acid positions Y374, Y435, and N437, wherein the amino acid position corresponds to SEQ ID NO:3.

Embodiment 37 includes the non-natural flavin-dependent oxidase of any one of embodiments 21 to 36, wherein the variation comprises a deletion of about 5 to about 50 amino acid residues at the N-terminus of SEQ ID NO:3.

Embodiment 38 includes the non-natural flavin-dependent oxidase of any one of embodiments 21 to 37, wherein the variation comprises a deletion of about 10 to about 40 amino acid residues at the N-terminus of SEQ ID NO:3.

Embodiment 39 includes the non-natural flavin-dependent oxidase of any one of embodiments 21 to 38, wherein the variation comprises a deletion of about 12 to about 35 amino acid residues at the N-terminus of SEQ ID NO:3.

Embodiment 40 includes the non-natural flavin-dependent oxidase of any one of embodiments 21 to 39, wherein the variation comprises a deletion of about 14 to about 29 amino acid residues at the N-terminus of SEQ ID NO:3.

Embodiment 41 includes the non-natural flavin-dependent oxidase of any one of embodiments 21, 22, or 25 to 38, wherein the variation comprises a deletion of about 14 amino acid residues at the N-terminus of SEQ ID NO:3.

Embodiment 42 includes the non-natural flavin-dependent oxidase of any one of embodiments 21, 22, or 25 to 38, wherein the variation comprises a deletion of about 29 amino acid residues at the N-terminus of SEQ ID NO:3.

Embodiment 43 includes the non-natural flavin-dependent oxidase of any one of embodiments 21 to 42, wherein the non-natural flavin-dependent oxidase converts CBGA to cannabichromenic acid (CBCA), tetrahydrocannabinolic acid (THCA), or both.

Embodiment 44 includes the non-natural flavin-dependent oxidase of embodiment 43, wherein the non-natural flavin-dependent oxidase converts CBGA to CBCA at about pH 4 to about pH 9.

Embodiment 45 includes the non-natural flavin-dependent oxidase of any one of embodiments 21 to 44, wherein the non-natural flavin-dependent oxidase converts CBGOA to cannabiorcichromenic acid (CBCOA).

Embodiment 46 includes the non-natural flavin-dependent oxidase of embodiment 45, wherein the non-natural flavin-dependent oxidase converts CBGOA to CBCOA at about pH 4 to about pH 9.

Embodiment 47 includes the non-natural flavin-dependent oxidase of any one of embodiments 21 to 46, wherein the non-natural flavin-dependent oxidase converts CBGVA to cannabichromevarinic acid (CBCVA).

Embodiment 48 includes the non-natural flavin-dependent oxidase of embodiment 47, wherein the non-natural flavin-dependent oxidase converts CBGVA to CBCVA at about pH 4 to about pH 9.

Embodiment 49 includes the non-natural flavin-dependent oxidase of any one of embodiments 21 to 48, wherein the non-natural flavin-dependent oxidase converts CBG to cannabichromene (CBC) at about pH 4 to about pH 9.

Embodiment 50 includes the non-natural flavin-dependent oxidase of any one of embodiments 1 to 49, wherein the non-natural flavin-dependent oxidase converts CBGO to cannabiorcichromene.

Embodiment 51 includes the non-natural flavin-dependent oxidase of any one of embodiments 1 to 50, wherein the non-natural flavin-dependent oxidase converts CBGV to cannabichromevarin.

Embodiment 52 includes the non-natural flavin-dependent oxidase of any of embodiments 1 to 51, further comprising an affinity tag, a purification tag, a solubility tag, or a combination thereof.

Embodiment 53 includes a polynucleotide comprising a nucleic acid sequence encoding the non-natural flavin-dependent oxidase of any one of embodiments 1 to 52.

Embodiment 54 includes a polynucleotide comprising: (a) a nucleic acid sequence encoding a polypeptide having at least 70%, at least 80%, at least 85%, or at least 90% sequence identity to SEQ ID NO:1, 3, 19, or 20; and (b) a heterologous regulatory element operably linked to the nucleic acid sequence, wherein: (i) the polypeptide having at least 70%, at least 80%, at least 85%, or at least 90% sequence identity to SEQ ID NO:1 comprises an amino acid substitution at position V136, S137, T139, L144, Y249, F313, Q353, or a combination thereof; or (ii) the polypeptide having at least 70%, at least 80%, at least 85%, or at least 90% sequence identity to SEQ ID NO:3 comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof; or (iii) the polypeptide having at least 70%, at least 80%, at least 85%, or at least 90% sequence identity to SEQ ID NO:3 comprises a deletion of about 5 to about 50 amino acid residues at an N-terminus of SEQ ID NO:3, and optionally further comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or (iv) wherein the polypeptide having at least 70%, at least 80%, at least 85%, or at least 90% sequence identity to SEQ ID NO:19 or 20 optionally comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

Embodiment 55 includes an expression construct comprising the polynucleotide of embodiment 53 or 54.

Embodiment 56 includes an engineered cell comprising the non-natural flavin-dependent oxidase of any one of embodiments 1 to 52, the polynucleotide of embodiment 53 or 54, the expression construct of embodiment 55, or a combination thereof.

Embodiment 57 includes the engineered cell of embodiment 56, further comprising a cannabinoid biosynthesis pathway enzyme.

Embodiment 58 includes the engineered cell of embodiment 57, wherein the cannabinoid biosynthesis pathway enzyme comprises olivetol synthase (OLS), olivetolic acid cyclase (OAC), prenyltransferase, or a combination thereof.

Embodiment 59 includes the engineered cell of embodiment 58, wherein the OLS comprises an amino acid substitution at position A125, S126, D185, M187, L190, G204, G209, D210, G211, G249, G250, L257, F259, M331, S332, or a combination thereof, wherein the position corresponds to SEQ ID NO:7.

Embodiment 60 includes the engineered cell of embodiment 59, wherein the amino acid substitution is selected from A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M1871, M187K, M187R, L190G, L190A, L190S, L190P, L190C, L190T, L190D, L190N, L190E, L190Q, L190H, L190V, L190M, L190I, L190K, L190R, G204A, G204C, G204P, G204V, G204L, G2041, G204M, G204F, G204W, G204S, G204T, G204Y, G204H, G204N, G204Q, G204D, G204E, G204K, G204R, G209A, G209C, G209P, G209V, G209L, G2091, G209M, G209F, G209W, G209S, G209T, G209Y, G209H, G209N, G209Q, G209D, G209E, G209K, G209R, D210A, D210C, D210P, D210V, D210L, D2101, D210M, D210F, D210W, D210S, D210T, D210Y, D210H, D210N, D210Q, D210E, D210K, D210R, G211A, G211C, G211P, G211V, G211L, G211I, G211M, G211F, G211W, G211S, G211T, G211Y, G211H, G211N, G211Q, G211D, G211E, G211K, G211R, G249A, G249C, G249P, G249V, G249L, G2491, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M331I, M331K, M331R, S332G, S332A, and a combination thereof.

Embodiment 61 includes the engineered cell of any one of embodiments 58 to 60, wherein the OAC comprises an amino acid substitution at position L9, F23, V59, V61, V66, E67, 169, Q70, I73, I74, V79, G80, F81, G82, D83, R86, W89, L92,194, V46, T47, Q48, K49, N50, K51, V46, T47, Q48, K49, N50, K51, or a combination thereof, wherein the position corresponds to SEQ ID NO: 8.

Embodiment 62 includes the engineered cell of any one of embodiments 58 to 61, wherein the prenyltransferase comprises an amino acid substitution at position V45, F121, T124, Q159, M160, Y173, S212, A230, T267, Y286, Q293, R294, L296, F300, or a combination thereof, wherein the position corresponds to SEQ ID NO:9.

Embodiment 63 includes the engineered cell of embodiment 61, wherein the amino acid substitution is selected from V451, V45T, F121V, T124K, T124L, Q159S, M160L, M160S, Y173D, Y173K, Y173P, Y173Q, S212H, A230S, T267P, Y286V, Q293H, R294K, L296K, L296L, L296M, L296Q, F300Y, and a combination thereof.

Embodiment 64 includes the engineered cell of any one of embodiments 57 to 63, further comprising a geranyl pyrophosphate (GPP) biosynthesis pathway enzyme.

Embodiment 65 includes the engineered cell of embodiment 63, wherein the GPP biosynthesis pathway comprises a mevalonate (MVA) pathway, a non-mevalonate (MEP) pathway, an alternative non-MEP, non-MVA GPP pathway, or a combination thereof.

Embodiment 66 includes the engineered cell of embodiment 64 or 65, wherein the GPP biosynthesis pathway enzyme is geranyl pyrophosphate synthase (GPPS), farnesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, or a combination thereof.

Embodiment 67 includes the engineered cell of any of embodiments 57 to 66, wherein the cell is a bacterial cell.

Embodiment 68 includes the engineered cell of embodiment 67, wherein the cell is an Escherichia coli cell.

Embodiment 69 includes a cell extract or cell culture medium comprising cannabigerolic acid (CBGA), cannabichromenic acid (CBCA), tetrahydrocannabinolic acid (THCA), cannabigerol (CBG), cannabichromene (CBC), cannabigerorcinic acid (CBGOA), cannabiorcichromenic acid (CBCOA), cannabigerivarinic acid (CBGVA), cannabichromevarinic acid (CBCVA), an isomer, analog or derivative thereof, or a combination thereof derived from the engineered cell of any one of embodiments 56 to 68.

Embodiment 70 includes a method of making a cannabinoid selected from CBCA, CBC, CBCOA, CBCVA, THCA, an isomer, analog or derivative thereof, or a combination thereof, comprising: culturing the engineered cell of any one of embodiments 56 to 68, isolating the cannabinoid from the cell extract or cell culture medium of embodiment 69, or both.

Embodiment 71 includes a method of making CBCA, THCA, or an isomer, analog or derivative thereof, comprising contacting CBGA with the non-natural flavin-dependent oxidase of any one of embodiments 1 to 52.

Embodiment 72 includes a method of making CBCOA or an isomer, analog or derivative thereof, or a combination thereof, comprising contacting CBGOA with the non-natural flavin-dependent oxidase of any one of embodiments 1 to 52.

Embodiment 73 includes a method of making CBCVA and/or an isomer, analog or derivative thereof, or a combination thereof, comprising contacting CBGVA with the non-natural flavin-dependent oxidase of any one of embodiments 1 to 52.

Embodiment 73 includes a method of making CBC or an analog or derivative thereof, comprising contacting comprising contacting CBG with the non-natural flavin-dependent oxidase of any one of embodiments 1 to 52.

Embodiment 75 includes the method of any one of embodiments 71 to 74, wherein the contacting occurs at about pH 4 to about pH 9.

Embodiment 76 includes the method of any one of embodiments 71 to 75, wherein the flavin-dependent oxidase comprises SEQ ID NO:3.

Embodiment 77 includes the method of any one of embodiments 71 to 76, wherein the method is performed in an in vitro reaction medium.

Embodiment 78 includes the method of embodiment 77, wherein the in vitro reaction medium comprises a surfactant.

Embodiment 79 includes the method of embodiment 78, wherein the surfactant is about 0.01% (v/v) to about 1% (v/v) of the in vitro reaction medium.

Embodiment 80 includes the method of embodiment 78 or 79, wherein the surfactant is 2-[4-(2,4,4-trimethylpentan-2-yl)phenoxyl]ethanol.

Embodiment 81 includes the method of any one of embodiments 77 to 80, wherein the in vitro reaction medium comprises about 0.1% (v/v) 2-[4-(2,4,4-trimethylpentan-2-yl)phenoxyl]ethanol.

Embodiment 82 includes a method of making an isolated non-natural flavin-dependent oxidase, comprising isolating the non-natural flavin-dependent oxidase expressed in the engineered cell of any one of embodiments 56 to 68.

Embodiment 83 includes an isolated non-natural flavin-dependent oxidase made by the method of embodiment 82.

Embodiment 84 includes a composition comprising a cannabinoid or an isomer, analog or derivative thereof obtained from the engineered cell of any one of embodiments 56 to 68, the cell extract of embodiment 69, or the method of any one of embodiments 70 to 82.

Embodiment 85 includes the composition of embodiment 84, wherein the cannabinoid is CBCA, THCA, CBCOA, CBCVA, CBC, or an isomer, analog or derivative thereof, or a combination thereof.

Embodiment 86 includes the composition of embodiment 85, wherein the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

Embodiment 87 includes the composition of any one of embodiments 84 to 86, wherein the composition is a therapeutic or medicinal composition.

Embodiment 88 includes the composition of any one of embodiments 84 to 87, wherein the composition is a topical composition.

Embodiment 89 includes the composition of any one of embodiments 84 to 87, wherein the composition is an edible composition.

Embodiment 90 includes a composition comprising: (a) a flavin-dependent oxidase, wherein the flavin-dependent oxidase does not comprise a disulfide bond, and wherein the non-natural flavin-dependent oxidase is capable of oxidative cyclization of a prenylated aromatic compound into a cannabinoid; and (b) a cannabinoid, the prenylated aromatic compound, or both.

Embodiment 91 includes a composition comprising: (a) the non-natural flavin-dependent oxidase of any one of embodiments 1 to 52; and (b) a cannabinoid, the prenylated aromatic compound, or both.

Embodiment 92 includes the composition of embodiment 90 or 91, wherein the cannabinoid or the prenylated aromatic compound is CBGA, CBGOA, CBGVA, CBG, CBCA, THCA, CBCOA, CBCVA, CBC, or an isomer, analog, or derivative thereof, or a combination thereof.

Embodiment 92 includes the composition of any one of embodiments 90 to 92, further comprising an enzyme in a cannabinoid biosynthesis pathway.

Embodiment 93 includes the composition of embodiment 92, wherein the cannabinoid biosynthesis pathway enzyme comprises olivetol synthase (OLS), olivetolic acid cyclase (OAC), prenyltransferase, or a combination thereof.

### Examples

### Methods

Unless otherwise specified, the Examples provided herein were performed according to the following methods.

Cloning. Synthetic genes for UniProtKB-A0A2E0XWX6 (referred to herein as “Cds_11170A”), TamL (UniProtKB-D3Y1I2), and EncM (UniProtKB-Q9KHK2) with optimized codons for expression in E. coli were ordered. The synthetic gene coding for Cds_11170A was designed such that it encodes a C-terminal 6× poly-histidine tag (SEQ ID NO:21), and the gene was cloned into a modified expression vector. The synthetic genes coding for TamL and EncM were cloned into the common expression vector pET28 such that they contained a 6× poly-histidine tag (SEQ ID NO:21) and thrombin cleave site at their N-termini. For EncM, a variant with threonine 139 mutated to valine (T139V) was also evaluated (UniProtKB-U6A1G7). The synthetic gene coding for Clz9 was cloned into an expression vector such that a gene fusion was created coding for a Clz9 fusion protein with the N-terminal maltose binding protein (MBP-Clz9) for improved solubility. The latter fusion protein also contained a 6× poly-histidine tag (SEQ ID NO:21) for purification purposes.

Enzyme Expression. The expression plasmids containing tamL, cds_11170A, or encM were used to transform E. coli BL21 (DE3). A single colony was used to inoculate a 20-mL preculture of LB media and grown overnight at 35° C. 10 ml of the preculture was used to inoculate 1 L LB medium in 2.8-L Fernbach flasks supplemented with the appropriate antibiotic and grown at 37° C. until the OD₆₀₀=0.6. In some cases, 10 mg/L riboflavin was added to the culture media. Protein expression was induced by the addition of 0.5 mM isopropyl β-thiogalactoside (IPTG) at 16° C. for 20 h. Cells were harvested by centrifugation at 8000 g for 10 min. Typically, cells pellets were frozen and stored at −20° C. until purification.

Enzyme Purification. Frozen cells were resuspended in 50 mL of 50 mM potassium phosphate buffer, pH 8.0 with 300 mM KCl and 10 mM imidazole supplemented with a Pierce Protease Inhibitor tablet, EDTA free. Cells were lysed by passage through a Microfluidics microfluidizer at 4° C. Cell debris was removed by centrifugation at 29,000 g for 40 min followed by syringe filtering with a 0.2 μm filter. The resulting solution was loaded onto a 5-mL HisTrap HP (GE Healthcare) nickel affinity column that had been pre-equilibrated with resuspension buffer using an ATKA HPLC system. Protein was eluted from the column using 50 mM potassium phosphate buffer, pH 8.0 with 300 mM KCl and 300 mM imidazole over a gradient of 15 column volumes. Fractions of 3.0 mLs were collected and tested for purity by SDS-polyacrylamide gel electrophoresis. Typically, proteins eluted between 6-12 column volumes. Fractions containing the purest samples were pooled and concentrated using an Amicon Ultra-15 Centrifugal Filter Unit with 30 kDa cutoff. Enzymes were exchanged into 50 mM potassium phosphate buffer, pH 8.0 with 300 mM KCl with 10% glycerol by overnight dialysis at 4° C. protein or three repeated concentration and resuspension in storage buffer steps using Amicon centrifugal filters. Final protein concentration was determined by Bradford assay (BioRad, Hercules, CA), estimated purity, and calculated molecular weight of the enzyme. Yields were 100 mg, 50 mg, 6 mg, and 5 mg per liter of LB media for TamL, Clz9, Cds_11170A, and EncM, respectively. Final concentrated enzyme solutions were bright yellow in color and the UV spectra of TamL and EncM match previously published data.

Enzyme In-vitro Assay. For enzymatic assays, 4 μL of concentrated enzyme was mixed with 20 μL of a 240 μM CBGA solution in either 100 mM Tris·HCl, pH 7.4, or 100 mM Na·citrate, pH 5.0. To the CBGA substrate solution, 0.1% Triton™ X-100 (Sigma-Aldrich) was included unless otherwise stated. Typically, reactions were incubated at 30° C. or 37° C. for 24 hrs. Reactions were mixed with 216 μL of 75% acetonitrile solution containing 0.1% formic acid and 1.2 μM diclofenac and 2 μM ibuprofen as internal standards. Precipitated protein was removed by centrifugation and supernatant loaded onto an HPLC/MS system. Cannabinoids were identified by comparison of retention time, mass, and fragmentation pattern to authentic cannabinoid standards. Structures were inferred for cannabinoids whose standards were not available by comparison of retention time and fragmentation pattern. Enzymatically formed cannabinoids were quantified by relative peak area versus peak area of known concentrations of cannabinoid standards.

Analytical Methods. All enzymes were tested for their ability to produce cannabinoids from CBGA, CBGVA, or CBGOA in 100 mM Tris buffer, pH 7.4, or Citrate buffer, pH 5.0. Cannabinoid products were detected using HPLC/MS/MS following the mass of 357 Da (M-1) and three common cannabinoid fragments: loss of water (−18 Da, 339 Da), loss of carboxylic acid (−44 Da, 313 Da) and loss of carboxylic acid (−44 Da) and terpenyl C9 side chain (−122 Da) yielding peak at 191 Da.

The structures of cannabinoid products observed in the enzymatic reactions were confirmed by LC/MS/MS (Thermo Vanquish™ LC). LC used the following conditions: A solvent gradient was performed as follows: Starting with 15% solvent B for 1 minute, then ramp up solvent B to 95% until 12 minutes and hold for 3 minutes until 15 minutes, then sharp return to 15% solvent B over a 0.1 minute period until 15.1 minutes, and re-equilibrate until 18 minutes. The whole gradient takes 18 minutes, and the column temperature is set at 40° C. (Solvent A: 0.1% Ammonium Acetate in H₂O, Solvent B: Acetonitrile). MS data was acquired using heated electrospray sample introduction method with subsequent ion detection and separation using Thermo Q Exactive™ instrument. Data was acquired using Polarity switching to get coverage for both positive and negative ions using identical settings. The acquired MS data used following settings: AGC target was set at 1.00×10⁶ ions with minimum IT of 100 ms over 1 microscan and the m/z range of 70 to 1050 m/z at 30000 resolution. The acquired MS/MS data used the following settings: AGC target was set at 1.00×10⁵ with dynamic ion exclusion over 3.0 sec with minimum IT of 80 ms over 1 microscan and the isolation window of 1 m/z at 17500 resolution. Normalized collision energy was stepped at 25, 35 and 45 eV.

### Example 1. Lysate Assay

The 57 uncharacterized BBE-like enzymes shown in Table 6 were tested in an in-vitro lysate assay. These BBE-like enzymes did not exhibit activity with CBGA or CBGOA.

TABLE 6
BBE-Like Enzymes for Lysate Assay

| UniProtKB | Microorganism |
|---|---|
| A0A1B2HZS0 | Lentzea guizhouensis |
| A0A3A3DF21 | Pseudoalteromonas sp. MSK9-3 |
| A0A2T0SRN9 | Geodermatophilus tzadiensis |
| A0A1I7C372 | Geodermatophilus amargosae A |
| A0A124H5G2 | Streptomyces curacoi |
| A0A2E6YAM7 | Gammaproteobacteria bacterium |
| A0A2N3UZH1 | Streptomyces sp. GP55 |
| A0A2W2G821 | Sphaerisporangium sp. 7K107 |
| A0A101J861 | Streptomyces regalis |
| A0A2E0XWX6 | Phycisphaerae bacterium |
| A0A1M5B0S0 | Litoreibacter ascidiaceicola |
| A0A1S1HLW5 | Sphingomonas haloaromaticamans |
| A0A1H5AQN2 | Bradyrhizobium erythrophlei MT12 |
| A0A1N6H3D0 | Bradyrhizobium erythrophlei GAS478 |
| A0A249T0Q2 | Chitinophaga sp. MD30 |
| A0A0N0UXK4 | bacterium 336/3 |
| A0A358SK05 | Actinobacterium |
| A0A2N8NZ04 | Streptomyces eurocidicus |
| A0A0N7F488 | Kibdelosporangium phytohabitans |
| A0A2C2VYJ5 | Bacillus cereus |
| A0A0F4QXW3 | Pseudoalteromonas rubra |
| A0A0K3B041 | Kibdelosporangium sp. MJ126-NF4 |
| A0A0K3B3N7 | Kibdelosporangium sp. MJ126-NF4 |
| A0A0K3BAZ6 | Kibdelosporangium sp. MJ126-NF4 |
| A0A0R3FH33_A0A0R3FH33_9MYCO | |
| A0A117PE42_A0A117PE42_9ACTN | |
| A0A154MC42_A0A154MC42_9PSEU | |
| A0A1C7CFP8_A0A1C7CFP8_9MICO | |
| A0A1E4EJR7_A0A1E4EJR7_9BACT | |
| A0A1H0RXB8_A0A1H0RXB8_9ACTN | |
| A0A1H7KI02_A0A1H7KI02_STRJI | |
| A0A1H8NFU8_A0A1H8NFU8_9ACTN | |
| A0A1H9S2L7_A0A1H9S2L7_9PSEU | |
| A0A119ZAG1_A0A1I9ZAG1_9NOCA | |
| A0A1K0GUC1_A0A1K0GUC1_9ACTN | |
| A0A1M7QXF4_A0A1M7QXF4_9ACTN | |
| A0A1S2PRY3_A0A1S2PRY3_9ACTN | |
| A0A1U9NUY0_A0A1U9NUY0_9ACTN | |
| A0A1V4DXI0_A0A1V4DXI0_9ACTN | |
| A0A1V9WU88_A0A1V9WU88_9ACTN | |
| A0A1Y5XFQ7_A0A1Y5XFQ7_KIBAR | |
| A0A209CUP2_A0A209CUP2_9ACTN | |
| A0A229GK89_A0A229GK89_9ACTN | |
| A0A260ICH0_A0A260ICH0_9NOCA | |
| A0A2K9NL77_A0A2K9NL77_9PROT | |
| A0A2S1Z3M3_A0A2S1Z3M3_9ACTN | |
| A0A2U2ENY4_A0A2U2ENY4_9ACTN | |
| A0A2W6E2K8_A0A2W6E2K8_9PSEU | |
| A0A358SM40_A0A358SM40_9ACTN | |
| A0A370H5R3_A0A370H5R3_9NOCA | |
| A0A397RPG8_A0A397RPG8_9BURK | |
| A0A3C1NDJ3_A0A3C1NDJ3_9RHIZ | |
| A0A3D9SRM9_A0A3D9SRM9_9ACTN | |
| D7BRT5_D7BRT5_STRBB | |
| D9XHS6_D9XHS6_STRVT | |
| E4MZ37_E4MZ37_KITSK | |
| Q21NE7_Q21NE7_SACD2 | |

### Example 2. TamL, Cds_11170A

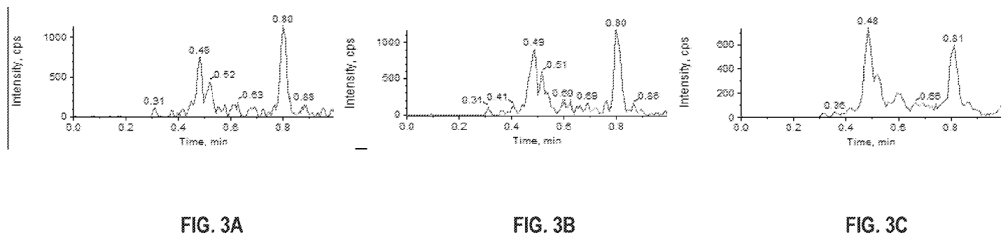

Neither TamL nor Cds_11170A showed cannabinoid products in a detectable amount that were not observed in a control reaction using E. coli BL21(DE3) empty vector lysate. FIG. 3 shows exemplary spectra for these reactions for the E. coli BL21(DE3) lysate control (FIG. 3A), purified TamL (FIG. 3B) and purified Cds_11170A (FIG. 3C). These results demonstrated that TamL and Cds_11170A wild type enzymes do not have inherent cannabinoid synthase activity under the conditions tested.

As shown in FIG. 1A, TamL and THCAS share structural similarity. Thus, a variant library of TamL was generated to combine the features of TamL and THCAS: (i) THCAS scaffold with N-terminal residues from TamL; and (ii) TamL scaffold with variations of its substrate binding site based on THCAS substrate binding site. The libraries were screened, but none of the variants had THCA synthase activity.

### Example 2. EncM and EncM T139V

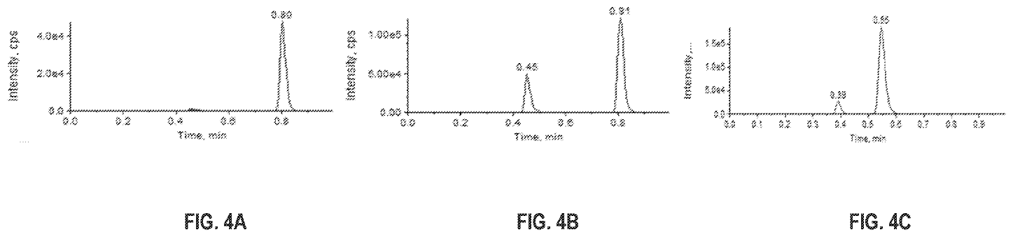

FIGS. 4A and 4B show the spectra of the in-vitro conversion of CBGA, and FIG. 4C shows the spectrum of the in-vitro conversion of CBGOA, at pH 7.4, using the T139V variant of EncM (“EncM T137V”). EncM T139V yielded a significant amount of cannabinoid product from both CBGA and CBGOA. The major peaks in FIGS. 4A and 4B eluted at a retention time of 0.80 min. Authentic cannabinoid standards showed that CBCA also elutes at 0.80 min. The ion fragmentation pattern of the peak at 0.80 min was identical to the ion fragmentation pattern of CBCA with the major fragment mass of 191 Da corresponding to the loss of CO₂ and the terpenyl C9 side chain. A comparison of the CBCA peak area produced from EncM T139V to the peak area of a known amount of CBCA standard showed that EncM T139V produced approximately 10 μM of CBCA in 24 hrs. Wild type EncM produced approximately 1 μM CBCA in 24 hrs. These results demonstrated that EncM and the EncM T139V variant surprisingly have inherent cannabinoid synthase activity, namely CBCA synthase activity. EncM T139V was also capable of converting CBGA to CBCA at pH 5.0, with lower activity than at pH 7.4.

When the solubility additive 0.1% Triton™ X-100 was not included the in vitro assay reaction with CBGA, a second detectable peak at pH 7.4 was observed with the retention time of 0.45 min (FIG. 4B). Based on the ion-fragmentation pattern and its molecular weight, the peak corresponds to an unknown cannabinoid-like compound.

EncM T139V was also active with CBGOA. As shown in FIG. 4C, the major peak resulting from CBGOA incubation with EncM T139V elutes at 0.55 min and is likely CBCOA based on its molecular weight and ion-fragmentation pattern, which were consistent with the expected molecular weight and ion-fragmentation pattern of CBCOA. An authentic standard of CBCOA was not available. Thus, EncM T139V surprisingly not only converts CBGA to CBCA but also CBGOA to CBCOA, indicating substrate promiscuity.

### Example 3. Clz9

Clz9 was tested with CBGA, CBGOA, CBGVA, and CBC as substrate.

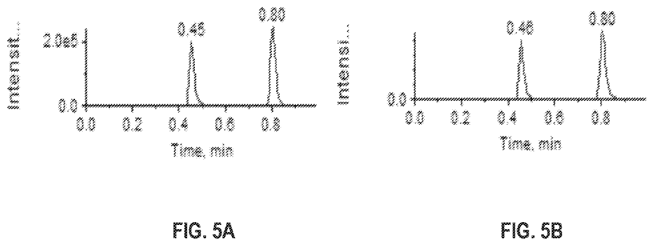

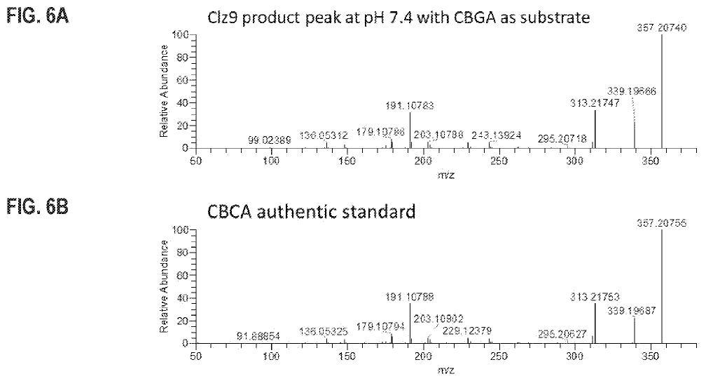

CBGA as substrate. The in vitro conversion of CBGA was evaluated with purified Clz9 protein as described above at pH 5.0 and at pH 7.4 in the presence or absence of the solubilizing agent Triton™-X100. FIGS. 5A and 5B show the LC/MS spectra of the in-vitro conversion of CBGA at pH 7.4 and at pH 5.5, respectively, in the presence of Triton™-X100. The LC/MS in the absence of Triton™-X100 were similar. The Clz9 reaction yielded a significant amount of cannabinoid product from CBGA at both pH. Two major peaks were observed, one with a retention time of 0.46 min and one with a retention time of 0.80 min. Authentic cannabinoid standards showed that CBCA also elutes at 0.80 min. The ion fragmentation pattern of the peak at 0.80 min was identical to the ion fragmentation pattern of CBCA (see FIG. 6) with the major fragment mass of 191 Da corresponding to the loss of CO₂ and the terpenyl C9 side chain. A comparison of the CBCA peak area produced from Clz9 to the peak area of a known amount of CBCA standard showed that Clz9 produced approximately 44 μM of CBCA from 200 uM CBGA in 2.5 hrs.

The ion-fragmentation pattern of the second peak with the retention time of 0.46 min suggested that it was an unknown cannabinoid-like compound, as neither the retention time nor the fragmentation pattern was consistent with CBDA or THCA. Without being bound by theory, it is hypothesized that the compound is an intermediate or side product from the Clz9 reaction with CBGA as substrate. For further structure elucidation, an orthogonal GC/MS method was used in which the compounds are derivatized before analysis with BSTFA/TMCS (99:1 Bis(trimethylsilyl)trifluoroacetamide/Trimethylchlorosilane). The ion fragmentation pattern of the derivatized compound in GC/MS was very similar to CBCA and indicated it possesses two hydroxyl groups like CBCA. FIG. 7 shows a putative reaction mechanism of Clz9 with CBGA as substrate. The CBCA-like compound may be derived by Clz9 oxidizing the hydroxyl group adjacent to the carboxyl group. If so, this CBCA-like compound can be converted to CBC via decarboxylation.

At both pH conditions with CBGA as substrate, Clz9 also showed an additional small peak with the retention time of 0.74 min. The ion fragmentation pattern of this peak with the major fragment mass of 313 Da corresponding to the loss of CO₂ indicates that Clz9 may also forms small amounts of THCA from CBGA.

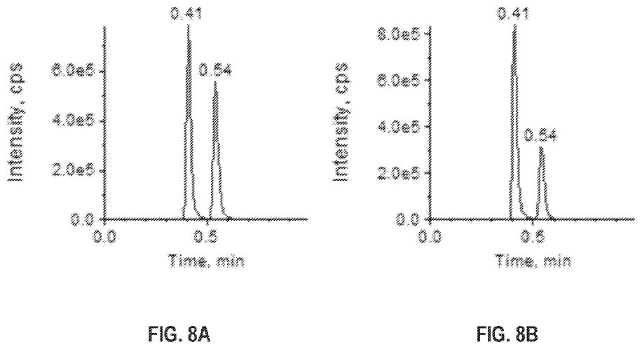

CBGOA as substrate. FIGS. 8A and 8B show the spectra of the in-vitro conversion of CBGOA at pH 7.4 and pH 5.0, respectively, with the solubility additive 0.1% Triton™ X-100 using Clz9 wild type enzyme. Clz9 showed the ability to yield a significant amount of cannabinoid product from CBGOA at both pH. One peak elutes at a retention time of 0.54 min. The molecular ion (301 Da) and the ion fragmentation pattern of the peak at 0.54 min (signature fragment at 135) suggested that it corresponds to CBCOA. An authentic standard for CBCOA was not available. A second peak elutes at a retention time of 0.41 min. The molecular ion (301 Da) and the ion fragmentation pattern of the peak at 0.41 min suggested that it may be a CBCOA-like compound, similar to the CBCA-like compound observed with CBGA as substrate. The peak height at 0.41 min increased at pH 5.0 as compared to pH 7.4.

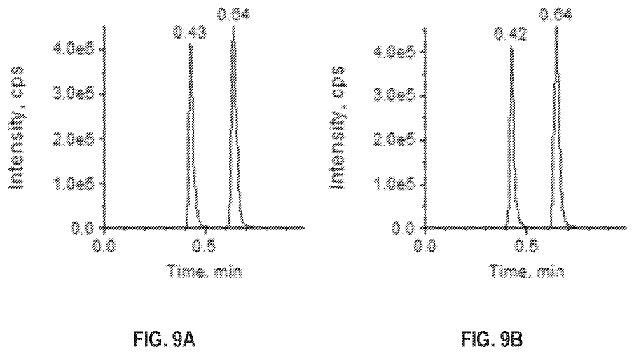

CBGVA as substrate. FIGS. 9A and 9B show the spectra of the in-vitro conversion of CBGVA at pH 7.4 and pH 5.0, respectively, with the solubility additive 0.1% Triton™ X-100 using Clz9 wild type enzyme. Clz9 showed the ability to yield a significant amount of cannabinoid product from CBGVA. One peak elutes at a retention time of 0.64 min. The molecular ion (329 Da) and the ion fragmentation pattern of the peak at 0.64 min (signature fragment at 163 Da) suggested that it corresponds to CBCVA. An authentic standard for CBCVA was not available. A second peak elutes at a retention time of 0.43 min. The molecular ion (329 Da) and the ion fragmentation pattern of the peak at 0.43 min suggested that it may be a CBCVA-like compound similar to the CBCA-like compound observed with CBGA as substrate.

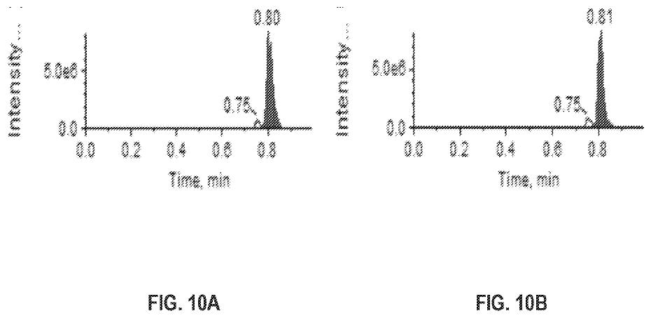

CBG as substrate. FIGS. 10A and 10B show the spectra of the in-vitro conversion of CBG (167 μM) at pH 7.4 and pH 5.0, respectively, with the solubility additive 0.1% Triton™ X-100 using Clz9 wild type enzyme. Clz9 showed the ability to yield a significant amount of cannabinoid product from CBG at both pH. In contrast to CBGA, CBGOA, and CBGVA as a substrate, only one major peak was observed, which eluted at a retention time of 0.8 min. An authentic CBC standard also eluted with retention time of 0.80. In addition, the molecular ion (315 Da) and the ion fragmentation pattern of the peak at 0.8 min (signature fragment at 193 Da) was identical to CBC. FIG. 12 shows a proposed reaction mechanism for Clz9 with CBG as substrate, indicating Clz9-catalyzed oxidations of both phenolic hydroxyl groups of CBG, leading to the formation of CBC after cyclization.

### Example 4. Identification of Clz9 Variants with Cannabinoid Synthase Activity

In this Example, a library of Clz9 variants were constructed and tested for cannabinoid synthase activity. Overnight cultures of E. coli BL21(DE3) containing plasmids expressing sequence-verified Clz9 variants were grown in 0.5 mL of LB media overnight at 35° C. in a 96-deep-well plate. On the following day, 10 μL of overnight culture was added to 1000 μL of LB media containing 100 μg/mL of carbenicillin in a 96-deep-well plate. The cultures were grown at 35° C. for 3 hours until OD₆₀₀ reached approximately 0.4 to 0.6, and 0.5 mM IPTG and 0.2 mM cumate were added to induce protein expression. Protein was expressed for approximately 18 to 20 hours at room temperature. Following expression, the culture OD was measured, and the cultures were transferred to 96 well plates. Cells were pelleted by centrifugation at 4000×g for 10 minutes. Cell pellets were resuspended to OD₆₀₀₌₂₀, and lysed using 50 mM Tris-HCl buffer, pH 7.4 with 50% SOLULYSE™ and protease inhibitor cocktail for 10 minutes. Cell lysates were clarified by centrifugation at 4000×g for 10 minutes. 4 μL of clarified lysate was mixed with 20 μL of 240 μM CBGA in 100 mM Tris-HCl buffer, pH 7.4, with 0.1% TRITON™ X-100 in 96-well plates. The plates were then sealed, and the reactions were incubated at 37° C. for 24 hours and then quenched with 376 μL of 75% acetonitrile solution containing 0.1% formic acid and 1.2 μM diclofenac and 2 μM ibuprofen as internal standards. Precipitated protein and cell debris were removed by vacuum filtration using a 0.2 μm 96-well filter plate (PALL). The flow through was directly injected into an LC/MS system for analysis. The spectra were monitored by LC/MS at 357/339 multiple reaction monitoring (MRM) transitions. Cannabinoid products were identified by retention time to authentic cannabinoid standards and quantified by relative peak area versus peak area of known concentrations of cannabinoid standards. Beside CBCA product, a CBCA-like “unknown” side product was also monitored.

Results of CBCA production by the Clz9 variant library are shown in Tables 3 to 6. Table 3 shows single mutants of Clz9 and their fold-improvement in CBCA production over wild-type Clz9. Table 4 shows further mutations of the Clz9 D404A variant, and their fold-improvement in CBCA production over the Clz9 D404A variant. The substitutions in Table 4 are indicated relative to the Clz9 D404A variant. Thus, for example, the variant indicated as “D404A+V323F” is equivalent to variant Clz9 D404S V323F variant.

Table 5 shows further mutations of the Clz9 D404A T438F variant, and their fold-improvement in CBCA production over the Clz9 D404A T438F variant. The substitutions in Table 5 are indicated relative to the Clz9 D404A T438F variant. Thus, for example, the variant indicated as “D404A T438F+N400W, A404S” is equivalent to variant Clz9 D404S T438F N400W variant.

TABLE 3
Clz9 Variants and CBCA Production

| | [CBCA] Production | Fold-improvement |
|---|---|---|
| Clz9 Variant | (μM) | over Clz9 WT |
| Clz9 WT | 1.27 | 1 |
| T438A | 6.43 | 5.05 |
| D404A | 6.155 | 4.83 |
| N400W | 2.345 | 1.83 |
| T438Y | 1.95 | 1.52 |

TABLE 4
Further Mutations to Clz9 D404A Variant and CBCA Production

| | | [CBCA] production | Fold-improvement |
|---|---|---|---|
| | Clz9 Variant | (μM) | over Clz9 D404A |
| | Clz9 D404A | 6.18 | 1 |
| D404A+ | V323F | 21.85 | 3.54 |
| | H402I | 20.55 | 3.33 |
| | T438F | 19.85 | 3.21 |
| | H402A | 16.70 | 2.70 |
| | H402V | 16.60 | 2.69 |
| | H402T | 15.75 | 2.55 |
| | L269T | 14.58 | 2.36 |
| | Y273R | 14.30 | 2.31 |
| | T438Y | 13.50 | 2.19 |
| | T438A | 11.22 | 1.82 |
| | N400W | 11.16 | 1.81 |
| | L269R | 10.27 | 1.66 |
| | Q275R | 9.03 | 1.46 |
| | V372I | 8.88 | 1.44 |
| | L283V | 8.45 | 1.37 |
| | C285L | 8.31 | 1.34 |
| | E370M | 8.30 | 1.34 |
| | E370Q | 8.24 | 1.33 |
| | H402M | 7.65 | 1.24 |

TABLE 5
Further Mutations to Clz9 D404A
T438F Variant and CBCA Production

| | | [CBCA] | Fold-improvement |
|---|---|---|---|
| | | production | over Clz9 D404A |
| | Clz9 Variant | (μM) | T438F |
| | Clz9 D404A T438F | 26.0 | 1 |
| D404A T438F+ | N400W | 67.95 | 2.62 |
| | V323F | 53.29 | 2.05 |
| | V323Y | 43.20 | 1.66 |
| | E370M | 36.26 | 1.40 |
| | H402I | 34.83 | 1.34 |
| | E370Q | 33.80 | 1.30 |
| | C285L | 31.54 | 1.22 |
| | N400W, A404S | 104.00 | 2.82 |
| | V323Y, A404S | 53.90 | 1.97 |
| | H402I, A404S | 33.10 | 1.28 |
| | E370Q, A404S | 32.00 | 1.23 |
| | V372I, N400W | 108.39 | 4.18 |
| | V323Y, N400W | 108.12 | 4.17 |
| | E370Q, N400W | 105.49 | 4.07 |
| | V323Y, E370M | 94.25 | 3.63 |
| | E370M, N400W | 92.83 | 3.58 |
| | V323F, H402I | 89.33 | 3.44 |
| | C285L, N400W | 84.90 | 3.27 |
| | V323F, N400W | 80.19 | 3.09 |
| | E370Q, H402T | 75.95 | 2.93 |
| | N400W, H402T | 75.25 | 2.90 |
| | V323F, H402T | 75.19 | 2.90 |
| | C285L, V323F | 72.32 | 2.79 |
| | L283V, N400W | 71.90 | 2.77 |
| | V323F, E370M | 70.40 | 2.71 |
| | Q275R, N400W | 65.25 | 2.51 |
| | V323Y, H402T | 65.05 | 2.51 |
| | V323F, V372I | 63.06 | 2.43 |
| | C285L, V323Y | 62.40 | 2.40 |
| | E370Q, H402I | 60.66 | 2.34 |
| | V323Y, E370Q | 59.01 | 2.27 |
| | Y273R, V323Y | 57.74 | 2.23 |
| | Y273R, N400W | 57.15 | 2.20 |
| | Y273R, V323F | 56.89 | 2.19 |
| | E370M, H402T | 56.47 | 2.18 |
| | L269T, N400W | 53.54 | 2.06 |
| | Q275R, V323Y | 51.09 | 1.97 |
| | V323Y, H402I | 46.62 | 1.80 |
| | V323F, E370Q | 45.77 | 1.76 |
| | Y273R, Q275R | 44.70 | 1.72 |
| | C285L, E370Q | 43.30 | 1.67 |
| | L283V, V323Y | 42.10 | 1.62 |
| | Y273R, H402I | 40.47 | 1.56 |
| | L269T, E370M | 38.00 | 1.46 |
| | C285L, H402T | 37.20 | 1.43 |
| | L269R, N400W | 36.52 | 1.41 |
| | Y273R, C285L | 35.00 | 1.35 |
| | L283V, H402I | 34.30 | 1.32 |
| | Q275R, E370Q | 33.90 | 1.31 |
| | V372I, H402I | 33.24 | 1.28 |
| | L283V, E370Q | 32.30 | 1.24 |
| | V372I, H402T | 31.93 | 1.23 |

Table 6 shows further mutations of the Clz9 D404A T438F N400W V323Y variant, and their fold-improvement in CBCA production over the Clz9 D404A T438F N400W V323Y variant. For the variants in Table 6, CBCA formation was measured at 30° C. after 3 hours, as compared to 37° C. after 24 hours as for Tables 3 to 5.

The substitutions in Table 6 are indicated relative to the Clz9 D404A T438F N400W V323Y variant. Thus, for example, the variant indicated as “D404A T438F N400W V323Y+E370Q” is equivalent to variant Clz9 D404A T438F N400W V323Y E370Q variant.

TABLE 6
Further Mutations to Clz9 D404A T438F
N400W V323Y Variant and CBCA Production

| | | [CBCA] | Fold-improvement |
|---|---|---|---|
| | | production | over Clz9 D404A |
| | Clz9 Variant | (μM) | T438F N400W V323Y |
| | Clz9 D404A | 0.97 | 1 |
| | N400W V323Y | | |
| D404A T438F | E370Q | 5.14 | 5.33 |
| N400W V323Y+ | V372I | 1.93 | 1.99 |
| | L269M | 1.69 | 1.75 |
| | C285L | 1.41 | 1.46 |
| | A281R | 1.33 | 1.38 |
| | I271H, E370Q | 7.10 | 7.36 |
| | E370Q, V372I | 6.58 | 6.82 |
| | L269M, E370Q | 4.95 | 5.12 |
| | C285L, E370Q | 4.25 | 4.40 |
| | Q275R, E370Q | 3.79 | 3.93 |
| | L283S, E370Q | 3.18 | 3.29 |
| | A281R, C285L | 3.18 | 3.29 |
| | Q275R, V372I | 2.89 | 2.99 |
| | C285L, E370M | 2.34 | 2.42 |
| | L269M, V372I | 2.33 | 2.41 |
| | Q275R, C285L | 2.26 | 2.34 |
| | I271H, L283S | 2.10 | 2.17 |
| | Q275R, A281R | 2.05 | 2.12 |
| | L269M, I271H | 2.03 | 2.10 |
| | I271H, E370M | 1.88 | 1.95 |
| | I271H, C285L | 1.84 | 1.90 |
| | A281R, V372I | 1.76 | 1.82 |
| | E370M, V372I | 1.71 | 1.77 |
| | L269M, Q275R | 1.63 | 1.68 |
| | C285L, V372I | 1.59 | 1.64 |
| | V372I, H402T | 1.43 | 1.48 |
| | L269M, E370M | 1.38 | 1.43 |
| | Q275R, E370M | 1.26 | 1.30 |
| | A281R, E370Q | 1.20 | 1.24 |
| | A281R, L283S | 1.19 | 1.23 |

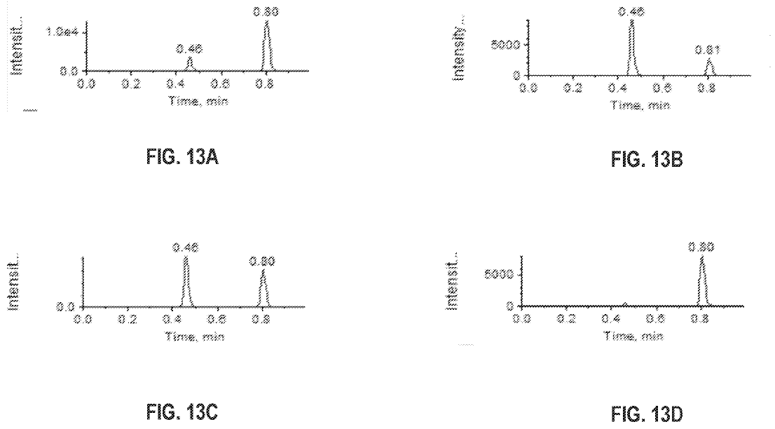

The Clz9 variants had varying levels of selectivity for CBCA synthesis. FIG. 13A shows the product profile of an in vitro reaction with Clz9 wild type and CBGA as substrate. As shown in FIG. 13A, the products included CBCA (peak at RT 0.880 min) and an unknown “CBCA-like” cannabinoid (peak at RT 0.46 min).

FIG. 13B shows the product profile of the Clz9 H402A variant. This variant formed significantly more of the “CBCA-like” cannabinoid than CBCA. A similar product profile was observed with the Clz9 H40AI, H402V, H402T, and H402M variants.

FIG. 13C shows the product profile of the Clz9 N400W variant. This variant formed slightly more of the “CBCA-like” cannabinoid than CBCA. A similar product profile was observed with the Clz9 V323F variant.

FIG. 13D shows the product profile of the Clz9 T438Y variant. This variant formed significantly less of the “CBCA-like” cannabinoid than CBCA. A similar product profile was observed with the Clz9 T438F and T438A variants. Thus, the Clz9 T438Y, T438F, and T438A variants are more selective for CBCA synthesis than wild type Clz9. All of the variants in Table 5, which included a mutation at T438, were also observed to form significantly less of the “CBCA-like” cannabinoid.

### Example 5. Evaluation of Truncated Clz9 Variants

In this example, the impact of truncating N-terminal amino acids on the activity of Clz9 was examined. Two truncated Clz9 variants were identified with over 2-fold higher activity than full length Clz9 protein.

Clz9 wildtype and the truncated Clz9 variants were expressed and purified using a HisTrap HP nickel affinity column as described in the Methods. The N-terminal His-tag was removed with thrombin via the encoded thrombin cleavage site. The in-vitro assay with CBGA as substrate was carried out as described in the Methods using 10 μM of purified Clz9 protein.

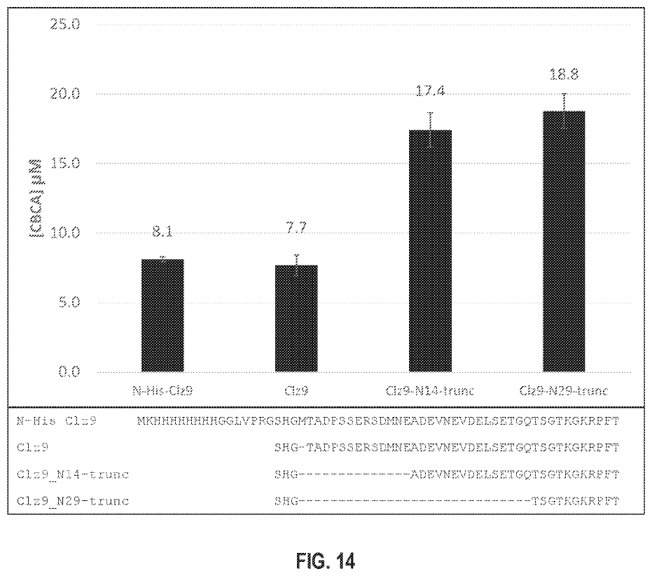

As shown in FIG. 14, cleaving the N-terminal His tag from the Clz9 protein did not have a significant impact on its CBCA synthase activity. However, both truncated Clz9 variants showed over two-fold higher specific CBCA synthase activity than full length Clz9. One variant had 14 and the other had 29 amino acids removed from the N-terminus of Clz9 (SEQ ID NO:19 and 20, respectively).

What is claimed is:

1. A non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid,
wherein:
(i) the non-natural flavin-dependent oxidase comprises at least 70% sequence identity to SEQ ID NO:1, and wherein the at least one amino acid variation comprises a substitution at position V136, S137, T139, L144, Y249, F313, Q353, or a combination thereof, wherein the amino acid position corresponds to SEQ ID NO:1; or
(ii) the non-natural flavin-dependent oxidase comprises at least 70% sequence identity to SEQ ID NO:3, and wherein the at least one amino acid variation comprises a substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or
(iii) the non-natural flavin-dependent oxidase comprises at least 70% sequence identity to SEQ ID NO:3, and wherein the at least one amino acid variation comprises a deletion of about 5 to about 50 amino acid residues at an N-terminus of SEQ ID NO:3 and optionally further comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or
(iv) the non-natural flavin-dependent oxidase comprises at least 70% sequence identity to SEQ ID NO:19 or 20 and optionally further comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, wherein the amino acid position corresponds to SEQ ID NO:3.

2. The non-natural flavin-dependent oxidase of claim 1, wherein the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG).

3. The non-natural flavin-dependent oxidase of claim 1 or 2, wherein the non-natural flavin-dependent oxidase has at least 80% sequence identity to SEQ ID NO:1, 3, 19, or 20.

4. The non-natural flavin-dependent oxidase of any one of claims 1 to 3, wherein the non-natural flavin-dependent oxidase is not glycosylated.

5. The non-natural flavin-dependent oxidase of any one of claims 1 to 4, wherein the non-natural flavin-dependent oxidase comprises a monovalently bound FAD cofactor, or wherein the non-natural flavin-dependent oxidase comprises a bivalently bound FAD cofactor.

6. The non-natural flavin-dependent oxidase of any one of claims 1 to 5, wherein the non-natural flavin-dependent oxidase is capable of oxidative cyclization of a prenylated aromatic compound into a cannabinoid at about pH 7.5.

7. The non-natural flavin-dependent oxidase of any one of claims 1 to 6, wherein catalytic activity of the non-natural flavin-dependent oxidase is substantially the same from about pH 5 to about pH 8.

8. The non-natural flavin-dependent oxidase of any one of claims 1 to 7, having at least 90% sequence identity to SEQ ID NO:1.

9. The non-natural flavin-dependent oxidase of claim 8, wherein the variation comprises an amino acid substitution selected from V136C, S137P, T139V, L144H, Y249H, F313A, Q353N, or a combination thereof, wherein the amino acid position corresponds to SEQ ID NO:1.

10. The non-natural flavin-dependent oxidase of claim 8 or 9, wherein the non-natural flavin-dependent oxidase converts CBGA to cannabichromenic acid (CBCA), tetrahydrocannabinolic acid (THCA), or both.

11. The non-natural flavin-dependent oxidase of claim 10, wherein the non-natural flavin-dependent oxidase converts CBGA to CBCA at about pH 4 to about pH 9.

12. The non-natural flavin-dependent oxidase of any one of claims 8 to 11, wherein the non-natural flavin-dependent oxidase converts CBGOA to cannabiorcichromenic acid (CBCOA);
wherein the non-natural flavin-dependent oxidase converts CBGVA to cannabichromevarinic acid (CBCVA); and/or
wherein the non-natural flavin-dependent oxidase converts CBG to cannabichromene (CBC).

13. The non-natural flavin-dependent oxidase of claim any one of claims 1 to 7, having at least 90% sequence identity to SEQ ID NO:3.

14. The non-natural flavin-dependent oxidase of claim any one of claims 1 to 7, having at least 90% sequence identity to SEQ ID NO:19 or 20.

15. The non-natural flavin-dependent oxidase of claim 13 or 14, wherein the variation comprises an amino acid substitution selected from W58Q, W58H, W58K, W58G, W58V, M101A, M101S, M101F, M101Y, L104M, L104H, I160V, G161C, G161A, G161Q, G161L, A163G, V167F, L168S, L168G, A171Y, A171F, N267V, N267M, N267L, L269M, L269T, L269A, L269R, I271H, I271R, Y2731, Y273R, Q275K, Q275R, A281R, L283V, C285L, E287H, E287L, V323F, V323Y, V336F, A338I, G340L, L342Y, E370M, E370Q, V372A, V372E, V372I, V372L, V372T, V372C, A398E, A398V, N400W, H402T, H402I, H402V, H402A, H402M, H402Q, D404S, D404T, D404A, V436L, T438A, T438Y, T438F, or a combination thereof, preferably wherein the variation comprises an amino acid substitution selected from T438A, T438Y, N400W, D404A, or a combination thereof, wherein the amino acid position corresponds to SEQ ID NO:3.

16. The non-natural flavin-dependent oxidase of claim 15, wherein the variation comprises an amino acid substitution at position D404 and an amino acid substitution at position L269, Y273, Q275, L283, C285, V323, E370, V372, N400, H402, T438, or a combination thereof, preferably wherein the variation comprises D404A and one of: L269R, L269T, Q275R, Y273R, L283V, C285L, V323F, V323Y, E370M, E370Q, V372I, N400W, H402A, H402I, H402M, H402T, H402V, T438A, T438F, or T438Y, wherein the amino acid position corresponds to SEQ ID NO:3.

17. The non-natural flavin-dependent oxidase of claim 15, wherein the variation comprises an amino acid substitution at position D404, an amino acid substitution at position T438, and an amino acid substitution at position L269, Y273, Q275, L283, C285, V323, E370, V372, N400, H402, T438, or a combination thereof, wherein the amino acid position corresponds to SEQ ID NO:3.

18. The non-natural flavin-dependent oxidase of claim 17, wherein the variation comprises:
a. D404A, T438F, and N400W;
b. D404A, T438F, and V323F;
c. D404A, T438F, and V323Y;
d. D404A, T438F, and E370M;
e. D404A, T438F, and H402I;
f D404A, T438F, and E370Q;
g. D404A, T438F, and C285L;
h. T438F, N400W, and D404S;
i. T438F, V323Y, and D404S;
j. T438F, H402I, and D404S;
k. T438F, E370Q, and D404S;
l. D404A, T438F, V372I, and N400W;
m. D404A, T438F, V323Y, and N400W;
n. D404A, T438F, E370Q, and N400W;
o. D404A, T438F, V323Y, and E370M;
p. D404A, T438F, E370M, and N400W;
q. D404A, T438F, V323F, and H402I;
r. D404A, T438F, C285L, and N400W;
s. D404A, T438F, V323F, and N400W;
t. D404A, T438F, E370Q, and H402T;
u. D404A, T438F, N400W, and H402T;
v. D404A, T438F, V323F, and H402T;
w. D404A, T438F, C285L, and V323F;
x. D404A, T438F, L283V, and N400W;
y. D404A, T438F, V323F, and E370M;
z. D404A, T438F, Q275R, and N400W;
aa. D404A, T438F, V323Y, and H402T;
bb. D404A, T438F, V323F, and V372I;
cc. D404A, T438F, C285L, and V323Y;
dd. D404A, T438F, E370Q, and H402I;
ee. D404A, T438F, V323Y, and E370Q;
ff. D404A, T438F, Y273R, and V323Y;
gg. D404A, T438F, Y273R, and N400W;
hh. D404A, T438F, Y273R, and V323F;
ii. D404A, T438F, E370M, and H402T;
jj. D404A, T438F, L269T, and N400W;
kk. D404A, T438F, Q275R, and V323Y;
ll. D404A, T438F, V323Y, and H402I;
mm. D404A, T438F, V323F, and E370Q;
nn. D404A, T438F, Y273R, and Q275R;
oo. D404A, T438F, C285L, and E370Q;
pp. D404A, T438F, L283V, and V323Y;
qq. D404A, T438F, Y273R, and H402I;
rr. D404A, T438F, L269T, and E370M;
ss. D404A, T438F, C285L, and H402T;
tt. D404A, T438F, L269R, and N400W;
uu. D404A, T438F, Y273R, and C285L;
vv. D404A, T438F, L283V, and H402I;
ww. D404A, T438F, Q275R, and E370Q;
xx. D404A, T438F, V372I, and H402I;
yy. D404A, T438F, L283V, and E370Q; or
zz. D404A, T438F, V372I, and H402T,
preferably wherein the variation comprises D404A, N400W, and V323Y; or D404A, T438F, N400W, and V323Y.

19. The non-natural flavin-dependent oxidase of claim 15, wherein the variation comprises an amino acid substitution at position D404, an amino acid substitution at position T438, an amino acid substitution at position N400, an amino acid substitution at position V323, and an amino acid substitution at position L269, I271, Q275, A281, L283, C285, E370, V372, H402, or a combination thereof, preferably wherein the variation comprises D404A, T438F, N400W, V323Y, and one or more of: L269M, I271H, Q275R, A281R, L283S, C285L, E370M, E370Q, V372I, and H402T.

20. The non-natural flavin-dependent oxidase of claim 19, wherein the variation comprises:
a. D404A, T438F, N400W, V323Y, and E370Q;
b. D404A, T438F, N400W, V323Y, and V372I;
c. D404A, T438F, N400W, V323Y, and L269M;
d. D404A, T438F, N400W, V323Y, and C285L;
e. D404A, T438F, N400W, V323Y, and A281R;
f D404A, T438F, N400W, V323Y, I271H, and E370Q;
g. D404A, T438F, N400W, V323Y, E370Q, and V372I;
h. D404A, T438F, N400W, V323Y, L269M, and E370Q;
i. D404A, T438F, N400W, V323Y, C285L, and E370Q;
j. D404A, T438F, N400W, V323Y, Q275R, and E370Q;
k. D404A, T438F, N400W, V323Y, L283S, and E370Q;
l. D404A, T438F, N400W, V323Y, A281R, and C285L;
m. D404A, T438F, N400W, V323Y, Q275R, and V372I;
n. D404A, T438F, N400W, V323Y, C285L, and E370M;
o. D404A, T438F, N400W, V323Y, L269M, and V372I;
p. D404A, T438F, N400W, V323Y, Q275R, and C285L;
q. D404A, T438F, N400W, V323Y, I271H, and L283S;
r. D404A, T438F, N400W, V323Y, Q275R, and A281R;
s. D404A, T438F, N400W, V323Y, L269M, and I271H;
t. D404A, T438F, N400W, V323Y, I271H, and E370M;
u. D404A, T438F, N400W, V323Y, I271H, and C285L;
v. D404A, T438F, N400W, V323Y, A281R, and V372I;
w. D404A, T438F, N400W, V323Y, E370M, and V372I;
x. D404A, T438F, N400W, V323Y, L269M, and Q275R;
y. D404A, T438F, N400W, V323Y, C285L, and V372I;
z. D404A, T438F, N400W, V323Y, V372I, and H402T;
aa. D404A, T438F, N400W, V323Y, L269M, and E370M;
bb. D404A, T438F, N400W, V323Y, Q275R, and E370M;
cc. D404A, T438F, N400W, V323Y, A281R, and E370Q; or
dd. D404A, T438F, N400W, V323Y, A281R, and L283S.

21. The non-natural flavin-dependent oxidase of any one of claims 14 to 20, wherein the non-natural flavin-dependent oxidase does not comprise a variation at any of amino acid positions Y374, Y435, and N437, wherein the amino acid position corresponds to SEQ ID NO:3.

22. The non-natural flavin-dependent oxidase of any one of claims 13 or 15 to 20, wherein the variation comprises a deletion of about 5 to about 50 amino acid residues, preferably a deletion of about 10 to about 40 amino acid residues, more preferably a deletion of about 12 to about 35 amino acid residues, or more preferably a deletion of about 14 to about 29 amino acid residues, at the N-terminus of SEQ ID NO:3.

23. The non-natural flavin-dependent oxidase of any one of claims 13 to 22, wherein the non-natural flavin-dependent oxidase converts CBGA to cannabichromenic acid (CBCA), tetrahydrocannabinolic acid (THCA), or both, optionally wherein the non-natural flavin-dependent oxidase performs one or more of the following conversions: CBGA to CBCA; CBGOA to CBCOA; CBGVA to CBCVA; CBG to CBC; CBGO to cannabiorcichromene; and/or CBGV to cannabichromevarin, preferably wherein the conversion is performed at about pH 4 to about pH 9.

24. The non-natural flavin-dependent oxidase of any of claims 1 to 23, further comprising an affinity tag, a purification tag, a solubility tag, or a combination thereof.

25. A polynucleotide comprising a nucleic acid sequence encoding the non-natural flavin-dependent oxidase of any one of claims 1 to 24.

26. A polynucleotide comprising: (a) a nucleic acid sequence encoding a polypeptide having at least 80% sequence identity to SEQ ID NO:1, 3, 19, or 20; and (b) a heterologous regulatory element operably linked to the nucleic acid sequence,
wherein:
(i) the polypeptide having at least 80% identity to SEQ ID NO:1 comprises an amino acid substitution at position V136, S137, T139, L144, Y249, F313, Q353, or a combination thereof; or
(ii) the polypeptide having at least 80% identity to SEQ ID NO:3 comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof; or
(iii) the polypeptide having at least 80% identity to SEQ ID NO:3 comprises a deletion of about 5 to about 50 amino acid residues at an N-terminus of SEQ ID NO:3, and optionally further comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3; or
(iv) the polypeptide having at least 80% sequence identity to SEQ ID NO:19 or 20 optionally comprises an amino acid substitution at position W58, M101, L104, I160, G161, A163, V167, L168, A171, N267, L269, I271, Y273, Q275, A281, L283, C285, E287, V323, V336, A338, G340, L342, E370, V372, A398, N400, H402, D404, V436, T438, or a combination thereof, wherein the amino acid position corresponds to SEQ ID NO:3.

27. An expression construct comprising the polynucleotide of claim 25 or 26.

28. An engineered cell comprising the non-natural flavin-dependent oxidase of any one of claims 1 to 24, the polynucleotide of claim 25 or 26, the expression construct of claim 27, or a combination thereof.

29. The engineered cell of claim 28, further comprising a cannabinoid biosynthesis pathway enzyme.

30. The engineered cell of claim 29, wherein the cannabinoid biosynthesis pathway enzyme comprises olivetol synthase (OLS), olivetolic acid cyclase (OAC), prenyltransferase, a geranyl pyrophosphate (GPP) biosynthesis pathway enzyme, or a combination thereof,
optionally wherein the OLS comprises an amino acid substitution at position A125, S126, D185, M187, L190, G204, G209, D210, G211, G249, G250, L257, F259, M331, S332, or a combination thereof, wherein the position corresponds to SEQ ID NO:7;
optionally wherein the OAC comprises an amino acid substitution at position L9, F23, V59, V61, V66, E67, 169, Q70, I73, I74, V79, G80, F81, G82, D83, R86, W89, L92, I94, V46, T47, Q48, K49, N50, K51, V46, T47, Q48, K49, N50, K51, or a combination thereof, wherein the position corresponds to SEQ ID NO:8; and
optionally wherein the prenyltransferase comprises an amino acid substitution at position V45, F121, T124, Q159, M160, Y173, S212, A230, T267, Y286, Q293, R294, L296, F300, or a combination thereof, wherein the position corresponds to SEQ ID NO:9.

31. The engineered cell of any of claims 28 to 30, wherein the cell is a bacterial cell, preferably wherein the cell is an Escherichia coli cell.

32. A cell extract or cell culture medium comprising cannabigerolic acid (CBGA), cannabichromenic acid (CBCA), tetrahydrocannabinolic acid (THCA), cannabigerol (CBG), cannabichromene (CBC), cannabigerorcinic acid (CBGOA), cannabiorcichromenic acid (CBCOA), cannabigerivarinic acid (CBGVA), cannabichromevarinic acid (CBCVA), an isomer, analog or derivative thereof, or a combination thereof derived from the engineered cell of any one of claims 28 to 31.

33. A method of making a cannabinoid selected from CBCA, CBC, CBCOA, CBCVA, THCA, an isomer, analog or derivative thereof, or a combination thereof, comprising: culturing the engineered cell of any one of claims 28 to 31, isolating the cannabinoid from the cell extract or cell culture medium of claim 32, or both.

34. A method of making a cannabinoid selected from CBCA, THCA, CBCOA, CBCVA, CBG, or an isomer, analog or derivative thereof, comprising:
contacting one or more of CBGA, CGBOA, CGBVA, and CBG with the non-natural flavin-dependent oxidase of any one of claims 1 to 24, preferably wherein the contacting occurs at about pH 4 to about pH 9.

35. The method of claim 34, wherein the method is performed in an in vitro reaction medium, optionally wherein the in vitro reaction medium comprises a surfactant, further optionally wherein the surfactant is about 0.01% (v/v) to about 1% (v/v) of the in vitro reaction medium, and preferably wherein the surfactant is 2-[4-(2,4,4-trimethylpentan-2-yl)phenoxy]ethanol.

36. A method of making an isolated non-natural flavin-dependent oxidase, comprising isolating the non-natural flavin-dependent oxidase expressed in the engineered cell of any one of claims 28 to 31.

37. An isolated non-natural flavin-dependent oxidase made by the method of claim 36.

38. A composition comprising a cannabinoid or an isomer, analog or derivative thereof obtained from the engineered cell of any one of claims 28 to 31, the cell extract of claim 32, or the method of any one of claims 33 to 36.

39. The composition of claim 38, wherein the cannabinoid is CBCA, THCA, CBCOA, CBCVA, CBC, or an isomer, analog or derivative thereof, or a combination thereof, optionally wherein the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

40. The composition of claim 38 or 39, wherein the composition is a therapeutic or medicinal composition; a topical composition; an edible composition; or a combination thereof.

41. A composition comprising: (a) the non-natural flavin-dependent oxidase of any one of claims 1 to 24; and (b) a cannabinoid, the prenylated aromatic compound, or both, preferably wherein the cannabinoid or the prenylated aromatic compound is CBGA, CBGOA, CBGVA, CBG, CBCA, THCA, CBCOA, CBCVA, CBC, or an isomer, analog, or derivative thereof, or a combination thereof optionally wherein the composition further comprises an enzyme in a cannabinoid biosynthesis pathway, preferably wherein the cannabinoid biosynthesis pathway enzyme comprises OLS, OAC, an enzyme in a geranyl pyrophosphate (GPP) pathway, prenyltransferase, or a combination thereof.


SEQUENCE LISTING (25 sequences)

SEQ ID NO: 1 - protein, 462 aa, Streptomyces sp.
MQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHTGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 2 - protein, 462 aa, Artificial Sequence
  Description of Artificial Sequence Synthetic polypeptide
MQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHVGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 3 - protein, 506 aa, Streptomyces sp.
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 4 - protein, 480 aa, Artificial Sequence
  Description of Artificial Sequence Synthetic polypeptide
MGSSHHHHHHSSGLVPRGSQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHTGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 5 - protein, 480 aa, Artificial Sequence
  Description of Artificial Sequence Synthetic polypeptide
MGSSHHHHHHSSGLVPRGSQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHVGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 6 - protein, 909 aa, Artificial Sequence
  Description of Artificial Sequence Synthetic polypeptide
MGSSHHHHHHGSSGASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSITSLYGSSGSSGSNLYFQSNGMTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 7 - protein, 385 aa, Streptomyces antibioticus
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 8 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 9 - protein, 305 aa, Unknown
  Description of Unknown Prenyltransferase sequence
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 10 - protein, 542 aa, Artificial Sequence
  Description of Artificial Sequence Synthetic polypeptide
MQACNNHQLTDAILQEFSQTLSGDLVLPTDGLYQWARLIHHTNFDGIYPVGIVFCETPEDVSKAILFARQFGLHVTARGGGHSYEGYSVTGGLLIDVSRMNSVSVNPAAMTAVVGAGAVLIDVYHGLYYPHKLSIPGGSCPSVGIAGYLLGGGVGLQSRTYGVGCDRVLEIGVVLASGEYVVASPTNHSDLYWAYRGGGGGNFGVVTHFKMQCHPVDRLSYAIITWDWEAAAPAFNAWQNWLKGLGEDYRNFSIFKFLVNAGGDGGLGPKVNLIVQFDSQSNTGTGELETLMAPLLNTAHEHIVSQTIMNGDFFEVTMEIMAGCAWPKTDLDTEFLRCHTVGNPAFPMASLPRDTYKAKSTFFADVISEKGIETCIKAIEDRFNSNLPDNSSQFTCALQFDSEGGIMGDVPKDATAFMHRDCLMHCQYLAYWPAEGDAWFDDNPDYSYCDVSNGSMQWISDAFNVLWPFGNGHAYQNYIDKEQPNWLYAYYGENVERLRTVKAKYDPDNIWKFEQSIPPAQGGSGGSGSGSGGSGSHHHHHH

SEQ ID NO: 11 - protein, 518 aa, Artificial Sequence
  Description of Artificial Sequence Synthetic polypeptide
MGSSHHHHHHSSGLVPRGSKHIDSVAPGDIRYEDLRRGENLRFVGDPEEIHLVGSAAEIEQVLSRAVRSGKRVAVRSGGHCYEDFVANSDVRVVMDMSRLSAVGFDEERGAFAVEAGATLGAVYKTLFRVWGVTLPGGACPDVGAGGHILGGGYGPLSRMHGSIVDYLHAVEVVVVDASGDARTVIATREPSDPNHDLWWAHTGGGGGNFGVVVRYWLRTAEADVPPEPGRLLPRPPAEVLLNTTVWPWEGLDEAAFARLVRNHGRWFEQNSGPDSPWCDLYSVLALTRSQSGALAMTTQLDATGPDAEKRLETYLAAVSEGVGVQPHSDTRRLPWLHSTRWPGIAGDGDMTGRAKIKAAYARRSFDDRQIGTLYTRLTSTDYDNPAGVVALIAYGGKVNAVPADRTAVAQRDSILKIVYVTTWEDPAQDPVHVRWIRELYRDVYADTGGVPVPGGAADGAYVNYPDVDLADEEWNTSGVPWSELYYKDAYPRLQAVKARWDPRNVFRHALSVRVPPA

SEQ ID NO: 12 - DNA, 1443 bp, Artificial Sequence
  Description of Artificial Sequence Synthetic polynucleotide
atgggcagcagccatcatcatcatcatcacagcagcggcctggtgccgcgcggcagccagttcccgcagttggaccctgctactttagccgcgttctccgcagctttccgcggcgaactgatttggccatccgatgcagactacgatgaagcgcgccgtatctggaacggtactatcgaccgccgtccggctctcatcgcgcgctgtactagcaccccggacgtggttgctgcggtttccttcgcgcgcaaatctggcctgcttgtggcggttcgtggcggtggccactctatggctggtcactcggtatgcgacggtggcattgtgattgacctgtctctgatgaactccatcaaagtatcccgccgtctgcgccgtgctcgtgcgcagggtggctgcctgctcggtgctttcgacactgctacccaggcgcatatgctcgcgaccccggctggcgtcgtttcccacaccggcctgggcggtctggttctgggtggcggtttcggctggctgtcacgtaaatatggcctgagcatcgacaacctgacctctgtagaaatcgtgaccgctgacggtggcgtgctgaccgcatccgatactgagaacccggacttattctgggctgttcgcggaggaggaggtaattttggcgtcgtaaccgctttcgaattcgacttacaccgcgtcggcccagttcgtttcgcatcgacctactatagcctcgatgaaggcccgcaggtcatccgtgcttggcgtgaccacatggcgacggcaccggatgaactgacctgggcgctctatctgcgtctggcgccgccactgccggaactgcctgcagacatgcacggcaaaccggttatctgcgcaatgtcttgctggattggtgacccacatgaaggtgaacgtcagttagaatctattctgcatgccggtaaaccgcacggcctgactaaagcgacccttccgtaccgcgcactgcaggcctattccttcccgggcgcagtcgttccggaccgtatctacactaaatccgggtacctgaatgagctgtccgacgaggcgaccgataccgtgcttgaacacgcagcggatatcgcgtcgccgttcactcaacttgaactgctctacctgggcggtgccgttgctcgtgttccagatgacgcgaccgcttatcctaaccgccagtctccgtttgtgaccaacctggcggcagcttggatggatccgaccgaggatgctcgtcacaccgcttgggcgcgcgaaggttaccgtgcgctggcgggccatctgtccggtggctacgttaactttatgaacccgggtgaggcggaccgtacccgtgaagcctacggcgcggcaaaatttgagcgcctgcagggcgtgaaagctaaatatgacccgactaacctgttccgtctgaatcagaacattccgccatcctag

SEQ ID NO: 13 - DNA, 1443 bp, Artificial Sequence
  Description of Artificial Sequence Synthetic polynucleotide
atgggcagcagccatcatcatcatcatcacagcagcggcctggtgccgcgcggcagccagttcccgcagttggaccctgctactttagccgcgttctccgcagctttccgcggcgaactgatttggccatccgatgcagactacgatgaagcgcgccgtatctggaacggtactatcgaccgccgtccggctctcatcgcgcgctgtactagcaccccggacgtggttgctgcggtttccttcgcgcgcaaatctggcctgcttgtggcggttcgtggcggtggccactctatggctggtcactcggtatgcgacggtggcattgtgattgacctgtctctgatgaactccatcaaagtatcccgccgtctgcgccgtgctcgtgcgcagggtggctgcctgctcggtgctttcgacactgctacccaggcgcatatgctcgcgaccccggctggcgtcgtttcccacgtgggcctgggcggtctggttctgggtggcggtttcggctggctgtcacgtaaatatggcctgagcatcgacaacctgacctctgtagaaatcgtgaccgctgacggtggcgtgctgaccgcatccgatactgagaacccggacttattctgggctgttcgcggaggaggaggtaattttggcgtcgtaaccgctttcgaattcgacttacaccgcgtcggcccagttcgtttcgcatcgacctactatagcctcgatgaaggcccgcaggtcatccgtgcttggcgtgaccacatggcgacggcaccggatgaactgacctgggcgctctatctgcgtctggcgccgccactgccggaactgcctgcagacatgcacggcaaaccggttatctgcgcaatgtcttgctggattggtgacccacatgaaggtgaacgtcagttagaatctattctgcatgccggtaaaccgcacggcctgactaaagcgacccttccgtaccgcgcactgcaggcctattccttcccgggcgcagtcgttccggaccgtatctacactaaatccgggtacctgaatgagctgtccgacgaggcgaccgataccgtgcttgaacacgcagcggatatcgcgtcgccgttcactcaacttgaactgctctacctgggcggtgccgttgctcgtgttccagatgacgcgaccgcttatcctaaccgccagtctccgtttgtgaccaacctggcggcagcttggatggatccgaccgaggatgctcgtcacaccgcttgggcgcgcgaaggttaccgtgcgctggcgggccatctgtccggtggctacgttaactttatgaacccgggtgaggcggaccgtacccgtgaagcctacggcgcggcaaaatttgagcgcctgcagggcgtgaaagctaaatatgacccgactaacctgttccgtctgaatcagaacattccgccatcctag

SEQ ID NO: 14 - DNA, 1521 bp, Streptomyces sp.
atgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtgtttggaatgaatgttttgcagcacgtccgaaagaaattgtttattgtgcagatacccgtgatgttgttcgtgcactgcgtgaagttcgtcagcgtggtggtccgtttcgtgttcgtagcggtggtcatagcatgagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggtttttgtccggaaattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccctgtttaccattgcatgggattgggatcgtggtgccgaagcaattaaagcatggcaagaatggctggcaaccgcagatggtcgcattaatacactgtttattgcatatccgcaggaccaggatatgtttgcagccctgggttgttttgaaggtgatgcagcagaactggaaccgctgattgcaccgctggttcatgcagttgaaccgaccgaaaaagttgcagaaaccatgccgtggattgaagcactgagctttgttgaaacaatgcagggtgaagccatgaaagcaaccagcgttcgtgcaaaaggtaatctgagttttgttaccgaaccgctgggtgatcgtgccgttgaagaaatcaaaaaagcactggcacaggcaccgagccatcgtgccgaagttgttctgtatggtttaggtggcgcagttgcagcaaaaggtcgtcgtgaaaccgcatttgttcatcgtgatgcaccggttgcgctgaattatcataccgattgggatgatgaagccgaagatgatctgaattttgcctggattcagaatctgcgtgcaagcgttgcagcacataccgaaggtcgcggtagctatgttaataccattgatctgaccgttgaacattggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgccgcacgtcagctgcgttag

SEQ ID NO: 15 - DNA, 2730 bp, Artificial Sequence
  Description of Artificial Sequence Synthetic polynucleotide
atgggtagcagccatcaccatcatcatcatggtagcagcggtgcaagcgaagaaggcaaactggttatttggattaatggcgataaaggctataatggtctggcagaagttggcaaaaaattcgaaaaagataccggcattaaagtgaccgttgaacatccggataaactggaagaaaaatttccgcaggttgcagcaaccggtgatggtccggatattatcttttgggcacatgatcgttttggtggttatgcacagagcggtctgctggcagaaattacaccggataaagcatttcaggacaaactgtatccgtttacctgggatgcagttcgctataacggtaaactgattgcatatccgattgcagttgaagcactgagcctgatctataacaaagatctgctgccgaatccgcctaaaacctgggaagaaattccggcactggataaagaactgaaagcaaaaggtaaaagcgcactgatgtttaatctgcaagaaccgtattttacctggcctctgattgcagcagatggtggctatgcattcaaatatgaaaacggcaaatacgatatcaaggatgttggtgttgataatgcgggtgcaaaagccggtctgacctttctggttgatctgatcaaaaacaaacacatgaatgccgataccgattatagcattgcagaagcagcatttaacaaaggtgaaaccgcaatgacaattaatggtccgtgggcatggtcaaatattgataccagcaaagtgaattatggtgttaccgttctgccgacatttaaaggtcagccgagcaaaccgtttgttggtgtgctgagcgcaggtattaatgcagcaagcccgaacaaagaactggcaaaagaatttctggaaaactatctgctgaccgatgaaggtctggaagcagtgaataaagataaaccgctgggtgcagttgcactgaaaagctatgaagaggaattagcaaaagatccgcgtattgcagccaccatggaaaatgcacagaaaggcgaaattatgccgaatattccgcagatgagcgcattttggtatgccgttcgtaccgcagtgattaatgccgcatcaggtcgtcagaccgttgatgaagccctgaaagatgcccagaccaatagcattaccagcctgtatggtagcagcggtagctcaggtagcaatctgtattttcagagcaatggcatgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtgtttggaatgaatgttttgcagcacgtccgaaagaaattgtttattgtgcagatacccgtgatgttgttcgtgcactgcgtgaagttcgtcagcgtggtggtccgtttcgtgttcgtagcggtggtcatagcatgagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggtttttgtccggaaattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccctgtttaccattgcatgggattgggatcgtggtgccgaagcaattaaagcatggcaagaatggctggcaaccgcagatggtcgcattaatacactgtttattgcatatccgcaggaccaggatatgtttgcagccctgggttgttttgaaggtgatgcagcagaactggaaccgctgattgcaccgctggttcatgcagttgaaccgaccgaaaaagttgcagaaaccatgccgtggattgaagcactgagctttgttgaaacaatgcagggtgaagccatgaaagcaaccagcgttcgtgcaaaaggtaatctgagttttgttaccgaaccgctgggtgatcgtgccgttgaagaaatcaaaaaagcactggcacaggcaccgagccatcgtgccgaagttgttctgtatggtttaggtggcgcagttgcagcaaaaggtcgtcgtgaaaccgcatttgttcatcgtgatgcaccggttgcgctgaattatcataccgattgggatgatgaagccgaagatgatctgaattttgcctggattcagaatctgcgtgcaagcgttgcagcacataccgaaggtcgcggtagctatgttaataccattgatctgaccgttgaacattggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgccgcacgtcagctgcgttag

SEQ ID NO: 16 - DNA, 1626 bp, Artificial Sequence
  Description of Artificial Sequence Synthetic polynucleotide
atgcaggcatgtaataatcatcagctgaccgatgcaatcctgcaagaattttcacagaccctgagcggtgatctggttctgccgaccgatggtctgtatcagtgggcacgtctgattcatcataccaattttgatggtatttatccggtgggcattgtgttttgtgaaacaccggaagatgttagcaaagcaattctgtttgcacgtcagtttggtctgcatgttaccgcacgtggtggtggtcatagttatgaaggttatagcgttaccggtggtctgctgattgatgttagccgtatgaatagcgttagcgttaatccggcagcaatgaccgcagttgttggtgccggtgcagttctgatcgatgtttatcatggcctgtattatccgcacaaactgagcattccaggtggtagctgtccgagcgttggtattgcaggttatctgcttggtggtggcgtgggtctgcagagccgtacctatggtgttggttgtgatcgtgttctggaaattggtgttgttctggcaagcggtgaatatgttgttgcaagcccgaccaatcatagcgatctgtattgggcatatcgtggtggcggtggtggtaattttggtgtggttacccactttaaaatgcagtgtcatccggttgatcgtctgagctatgcaattattacctgggattgggaagcagcagcaccggcatttaatgcatggcagaattggctgaaaggtctgggtgaagattatcgcaatttctcgatctttaagttcctggttaatgcaggcggtgatggtggtctgggtccgaaagttaatctgattgttcagtttgatagccagagcaataccggcaccggtgaactggaaaccctgatggcaccgctgctgaataccgcacatgaacatattgttagccagaccattatgaacggcgatttttttgaagtgaccatggaaattatggcaggttgtgcatggccgaaaaccgatctggataccgaatttctgcgttgtcataccgttggcaatccggcatttccgatggcaagcctgcctcgtgatacctataaagcaaaaagcaccttttttgccgatgtgattagcgaaaaaggtatcgaaacctgcatcaaagccattgaagatcgctttaatagcaatctgccggataatagcagccagtttacctgtgcactgcagttcgatagcgaaggtggtattatgggtgatgttccgaaagatgcaaccgcatttatgcatcgtgattgtctgatgcattgtcagtatctggcatattggcctgccgaaggtgatgcatggtttgatgataacccggattatagctattgtgatgtgagcaatggtagcatgcagtggattagtgatgcctttaatgttctgtggccgtttggtaatggtcatgcatatcagaactatatcgataaagaacagccgaactggctgtatgcatattatggtgaaaatgttgaacgtctgcgtaccgtgaaagcaaaatatgatccggataacatctggaagtttgaacagagcattccgcctgcatagggcggtagcggaggatctggtagtggctctggaggatctggtagtcaccatcatcatcaccat

SEQ ID NO: 17 - DNA, 1557 bp, Artificial Sequence
  Description of Artificial Sequence Synthetic polynucleotide
atgggcagcagccatcatcatcatcatcacagcagcggcctggtgccgcgcggcagcaaacacattgactcggttgcgccgggagacatccgttacgaggatttgcgccgcggtgaaaacttacgtttcgttggtgatccagaggaaattcacttggtcggttcggcagcggaaattgaacaggttcttagtcgcgcggtgcgcagtgggaaacgtgtcgcggtacgctctggcgggcattgctacgaagattttgttgcgaacagcgacgtgcgcgtggttatggatatgtcccgcttaagcgcagtgggctttgacgaggagcgcggcgcttttgccgtagaggctggggccacgctgggcgcagtatacaagaccttgtttcgcgtatggggagtgactctgccaggtggcgcctgtcctgatgtaggcgctggcggacatatccttggcggtggatatggtcctttgtcacgcatgcatgggtcgatcgtcgattatcttcacgctgttgaggtggtcgtcgtcgacgcttctggagacgcccgtactgtaatcgctacccgcgagccgagcgaccctaaccacgatttatggtgggcgcacactggaggtggtggtgggaacttcggggtagtcgtacgctactggcttcgtacagcggaggccgacgtacctccggagcctgggcgcctgttgccccgtccaccagctgaagtcttgctgaatactacagtgtggccctgggagggattagacgaggccgcgtttgctcgtctggtgcgtaatcacggccgttggttcgaacaaaactcgggacctgattcgccttggtgtgacctttatagtgtcttagcgttgacccgctcacagtccggtgcgttagctatgacaactcagcttgacgcaacgggacctgacgccgaaaaacgtcttgagacatatcttgcggctgttagcgaaggcgttggggttcagccccattccgatacacgccgcttgccctggttgcattcgacacgctggccaggcatcgccggcgatggcgacatgacgggacgtgcgaagattaaggctgcctatgcccgtcgcagttttgacgatcgccaaattggcacattatacacacgtctgacgagcaccgattatgataaccctgctggagtcgtggctctgattgcttatggcggaaaagtcaacgcagtacctgccgaccgtactgccgtagcgcagcgcgattccattctgaagattgtatatgttacgacttgggaagaccccgctcaagatcctgtgcatgtgcgttggatccgcgagttataccgtgacgtttacgccgacacggggggtgtgcctgttcctgggggtgcggccgatggagcttacgttaactaccccgacgtggatttggcggacgaggaatggaatacttcgggggtcccgtggagcgagttatattacaaggatgcctatcctcgtctgcaagctgtgaaagcacgctgggacccgcgcaacgttttccgtcacgctttgtctgtccgtgtcccgccagcttag

SEQ ID NO: 18 - protein, 525 aa, Artificial Sequence
  Description of Artificial Sequence Synthetic polypeptide
MKHHHHHHHHGGLVPRGSHGTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 19 - protein, 492 aa, Artificial Sequence
  Description of Artificial Sequence Synthetic polypeptide
ADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 20 - protein, 477 aa, Artificial Sequence
  Description of Artificial Sequence Synthetic polypeptide
TSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 21 - protein, 6 aa, Artificial Sequence
  Description of Artificial Sequence Synthetic 6xHis tag
HHHHHH

SEQ ID NO: 22 - protein, 60 aa, Artificial Sequence
  Description of Artificial Sequence Synthetic polypeptide
MKHHHHHHHHGGLVPRGSHGMTADPSSERSDMNEADEVNEVDELSETGQTSGTKGKRPFT

SEQ ID NO: 23 - protein, 42 aa, Artificial Sequence
  Description of Artificial Sequence Synthetic polypeptide
SHGTADPSSERSDMNEADEVNEVDELSETGQTSGTKGKRPFT

SEQ ID NO: 24 - protein, 29 aa, Artificial Sequence
  Description of Artificial Sequence Synthetic peptide
SHGADEVNEVDELSETGQTSGTKGKRPFT

SEQ ID NO: 25 - protein, 14 aa, Artificial Sequence
  Description of Artificial Sequence Synthetic peptide
SHGTSGTKGKRPFT